(19) United States
(12) Patent Application Publication
(10) Pub. No.: US 2026/0191609 A1
(43) Pub. Date: Jul. 9, 2026

(54) AUTONOMOUS DRIVING OR NAVIGATION FOR A CONTINUUM ROBOT OR ENDOSCOPIC DEVICE OR SYSTEM

(71) Applicant: Canon U.S.A., Inc., Melville, NY (US)

(72) Inventor: Brian Ninni, Woburn, MA (US)

(21) Appl. No.: 19/438,348

(22) Filed: Dec. 31, 2025

Related U.S. Application Data
(60) Provisional application No. 63/742,346, filed on Jan. 6, 2025.

(51) Int. Cl. A61B 34/32; A61B 1/00; A61B 1/005; A61B 1/05; A61B 1/267; A61B 34/00; A61B 34/10; A61B 34/20; A61B 34/30; A61M 25/01

(52) U.S. Cl. CPC A61B 34/32; A61B 1/000096; A61B 1/00045; A61B 1/0016; A61B 1/0055; A61B 1/0057; A61B 1/05; A61B 1/2676; A61B 2034/107; A61B 2034/2065; A61B 2034/301; A61B 2034/742; A61M 25/0113

(57) ABSTRACT

One or more devices, systems, methods, and storage mediums for performing navigation planning, autonomous driving/navigation, movement detection, and/or control for a continuum robot are provided herein. Examples of such planning, autonomous navigation, movement detection, and/or control include, but are not limited to, navigation planning and/or autonomous navigation of one or more portions of a continuum robot towards a particular target, movement detection of the continuum robot, Follow-The-Leader smoothing, and/or state change(s) for a continuum robot. Examples of applications include imaging, evaluating, and diagnosing biological objects, such as, but not limited to, for Gastro-intestinal, cardio, bronchial, and/or ophthalmic applications, and being obtained via one or more optical instruments, such as, but not limited to, optical probes, catheters, endoscopes, and bronchoscopes. Techniques provided herein also improve processing and imaging efficiency while achieving images that are more precise, and also achieve reduced mental and physical burden and improved ease of use.

### CROSS-REFERENCE TO RELATED APPLICATION(S)

This application relates, and claims priority, to U.S. Prov. Patent Application Ser. No. 63/742,346, filed Jan. 6, 2025, the disclosure of which is incorporated by reference herein in its entirety.

### FIELD OF THE DISCLOSURE

The present disclosure generally relates to imaging and, more particularly, to bronchoscope(s), robotic bronchoscope(s), robot apparatus(es), method(s), and storage medium(s) that operate to image a target, object, or specimen (such as, but not limited to, a lung, a biological object or sample, tissue, etc.) and/or to a continuum robot apparatus, method, and storage medium to implement robotic control for all sections of a catheter or imaging device/apparatus or system to perform navigation planning and/or autonomous driving or navigation and/or to match a state or states when each section reaches or approaches a same or similar, or approximately a same or similar, state or states of a first section of the catheter or imaging device, apparatus, or system. One or more bronchoscopic, endoscopic, medical, camera, catheter, or imaging devices, systems, and methods and/or storage mediums for use with same, are discussed herein. One or more devices, methods, or storage mediums may be used for medical applications and, more particularly, to steerable, flexible medical devices that may be used for or with guide tools and devices in medical procedures, including, but not limited to, endoscopes, cameras, and catheters.

### BACKGROUND

Endoscopy, bronchoscopy, catheterization, and other medical procedures facilitate the ability to look inside a body. During such a procedure, a flexible medical tool may be inserted into a patient's body, and an instrument may be passed through the tool to examine or treat an area inside the body. For example, a bronchoscope is an endoscopic instrument to view inside the airways of a patient. Catheters and other medical tools may be inserted through a tool channel in the bronchoscope to provide a pathway to a target area in the patient for diagnosis, planning, medical procedure(s), treatment, etc.

Robotic bronchoscopes, robotic endoscopes, or other robotic imaging devices may be equipped with a tool channel or a camera and biopsy tools, and such devices (or users of such devices) may insert/retract the camera and biopsy tools to exchange such components. The robotic bronchoscopes, endoscopes, or other imaging devices may be used in association with a display system and a control system.

An imaging device, such as a camera, may be placed in the bronchoscope, the endoscope, or other imaging device/system to capture images inside the patient and to help control and move the bronchoscope, the endoscope, or the other type of imaging device, and a display or monitor may be used to view the captured images. An endoscopic camera that may be used for control may be positioned at a distal part of a catheter or probe (e.g., at a tip section).

The display system may display, on the monitor, an image or images captured by the camera, and the display system may have a display coordinate used for displaying the captured image or images. In addition, the control system may control a moving direction of the tool channel or the camera. For example, the tool channel or the camera may be bent according to a control by the control system. The control system may have an operational controller (such as, but not limited to, a joystick, a gamepad, a controller, an input device, etc.), and physicians may rotate or otherwise move the camera, probe, catheter, etc. to control same. However, such control methods or systems are limited in effectiveness. Indeed, while information obtained from an endoscopic camera at a distal end or tip section may help decide which way to move the distal end or tip section, such information does not provide details on how the other bending sections or portions of the bronchoscope, endoscope, or other type of imaging device may move to best assist the navigation.

At least one application is looking inside the body relates to lung cancer, which is the most common cause of cancer-related deaths in the United States. It is also a commonly diagnosed malignancy, second only to breast cancer in women and prostate cancer in men. Early diagnosis of lung cancer is shown to improve patient outcomes, particularly in peripheral pulmonary nodules (PPNs). During a procedure, such as a transbronchial biopsy, targeting lung lesions or nodules may be challenging. Lately, Electromagnetically Navigated Bronchoscopy (ENB) is increasingly applied in the transbronchial biopsy of PPNs due to its excellent safety profile, with fewer pneumothoraxes, chest tubes, significant hemorrhage episodes, and respiratory failure episodes than a CT-guided biopsy strategy (see e.g., as discussed in C. R. Dale, D. K. Madtes, V. S. Fan, J. A. Gorden, and D. L. Veenstra, “Navigational bronchoscopy with biopsy versus computed tomography-guided biopsy for the diagnosis of a solitary pulmonary nodule: a cost-consequences analysis,” J Bronchology Interv Pulmonol, vol. 19, no. 4, pp. 294-303, October. 2012, doi: 10.1097/LBR.0B013E318272157D, which is incorporated by reference herein in its entirety). However, ENB has lower diagnostic accuracy or value due to dynamic deformation of the tracheobronchial tree by bronchoscope maneuvers (see e.g., as discussed in T. Whelan, R. F. Salas-Moreno, B. Glocker, A. J. Davison, and S. Leutenegger, “ElasticFusion,” International Journal of Robotics Research, vol. 35, no. 14, pp. 1697-1716, December 2016, doi: 10.1177/0278364916669237, which is incorporated by reference herein in its entirety) and module motion due to the breathing motion of the lung (see e.g., as discussed in A. Chen, N. Pastis, B. Furukawa, and G. A. Silvestri, “The effect of respiratory motion on pulmonary nodule location during electromagnetic navigation bronchoscopy,” Chest, vol. 147, no. 5, pp. 1275-1281, May 2015, doi: 10.1378/CHEST.14-1425, which is incorporated by reference herein in its entirety). Robotic-assisted biopsy has emerged as a minimally invasive and precise approach for obtaining tissue samples from suspicious pulmonary lesions in lung cancer diagnosis. However, the reliance on human operators to guide a robotic system introduces potential variability in sampling accuracy and operator-dependent outcomes. Such operators may introduce human error, reduce efficiency of using a robotic system, have a steeper learning curve to using a robotic system, and affect surgeries as a result.

Vision-based tracking (VNB), as opposed to ENB, has been proposed to address the aforementioned issue of CT-to-body divergence (see e.g., as discussed in D. J. Mirota, M. Ishii, and G. D. Hager, “Vision-Based Navigation in Image-Guided Interventions,” https://doi.org/10.1146/annurev-bioeng-071910-124757, vol. 13, pp. 297-319, July 2011, doi: 10.1146/ANNUREV-BIOENG-071910-124757, which is incorporated by reference herein in its entirety). Vision-based tracking in VNB does not require an electromagnetic tracking sensor to localize the bronchoscope in CT; rather, VNB directly localizes the bronchoscope using the camera view, conceptually removing the chance of CT-to-body divergence.

Depth estimation was proposed as an alternative method of VNB to further reduce the CT-to-body divergence and overcome the intensity-based image registration drawbacks (see e.g., as discussed in M. Shen, S. Giannarou, and G. Z. Yang, “Robust camera localisation with depth reconstruction for bronchoscopic navigation,” Int J ComputAssist Radiol Surg, vol. 10, no. 6, pp. 801-813, June 2015, doi: 10.1007/511548-015-1197-Y, which is incorporated by reference herein in its entirety).

Alternatively, autonomous navigation in robotic guided bronchoscopy is a relative new concept. Sganga et al. (as discussed in J. Sganga, D. Eng, C. Graetzel, and D. B. Camarillo, “Autonomous Driving in the Lung using Deep Learning for Localization,” July 2019, Accessed: Jun. 28, 2023. [Online]. Available: https://arxiv.org/abs/1907.08136v1, which is incorporated by reference herein in its entirety) proposed the first attempt to autonomously navigate through the lung airways having as primary focus to improve the intraoperative registration between CT and live images and then attempt to autonomously navigate to the target. The Sganga, et al. method was limited to only 4 airways. However, the Sganga, et al. method requires a great co-registration between the live image and the pre-operative CT scan which can be detrimentally affected by the same drawbacks as VNB.

Other efforts have been made not to autonomously navigate through the airways but to automatically control the catheter tensioning system. Jaeger, et al. (as discussed in H. A. Jaeger et al., “Automated Catheter Navigation With Electromagnetic Image Guidance,” IEEE Trans Biomed Eng, vol. 64, no. 8, pp. 1972-1979, August 2017, doi: 10.1109/TBME.2016.2623383, which is incorporated by reference herein in its entirety) proposed such a method where Jaeger, et al. incorporated a custom tendon-driven catheter design with Electro-magnetic (EM) sensors controlled with an electromechanical drive train. However, the system needed heavy user interaction as the clinician uses a computer interfaced joystick to manipulate the catheter. A semi-automatic navigation of the biopsy needle during bronchoscopy was proposed by Kuntz, et al. (as discussed in A. Kuntz et al., “Autonomous Medical Needle Steering In Vivo,” November 2022, Accessed: Jun. 28, 2023. [Online]. Available: https://arxiv.org/abs/2211.02597v1, which is incorporated by reference herein in its entirety). The method uses pre-operative CT scans (3D) and EMC sensors to co-register cloud points of the nodule and live guidance. Nevertheless, the method cannot be used to navigate the bronchoscopic catheter into the airways, which is a critical step for reaching the nodules. Moreover, similar methods (as discussed in S. Chen, Y. Lin, Z. Li, F. Wang, and Q. Cao, “Automatic and accurate needle detection in 2D ultrasound during robot-assisted needle insertion process,” Int J Comput Assist Radiol Surg, vol. 17, no. 2, pp. 295-303, February 2022, doi: 10.1007/S11548-021-02519-6/FIGURES/8, which is incorporated by reference herein in its entirety) allow automatic localization of the needle in real-time without the need of an automatic needle navigation.

As such, there is a need for devices, systems, methods, and/or storage mediums that provide the feature(s) or details on how the other bending sections or portions of such imaging devices, imaging systems, etc. (e.g., endoscopic devices, bronchoscopes, other types of imaging devices/systems, etc.) may move to best assist navigation and/or state or state(s) for same, to keep track of a path of a tip of the imaging devices, imaging systems, etc., and there is a need for a more appropriate navigation of a device (such as, but not limited to, a bronchoscopic catheter being navigated to reach a nodule). Additionally, there is a need to reduce choppy and less refined motion that may occur from driving or navigating a catheter using stage insertion at a different time from driving or navigating the catheter via bending the catheter.

Accordingly, it would be desirable to provide at least one imaging, optical, or control device, system, method, and storage medium for controlling one or more endoscopic or imaging devices or systems, for example, by implementing automatic (e.g., robotic) or manual control of each portion or section of the at least one imaging, optical, or control device, system, method, and storage medium to keep track of and to match the state or state(s) of a first portion or section in a case where each portion or section reaches or approaches a same or similar, or approximately same or similar, state or state(s) and to provide a more appropriate navigation of a device (such as, but not limited to, a bronchoscopic catheter being navigated to reach a nodule).

### SUMMARY

Accordingly, it is a broad object of the present disclosure to provide imaging (e.g., computed tomography (CT), Magnetic Resonance Imaging (MRI), etc.) apparatuses, systems, methods, and storage mediums for using a navigation and/or control method or methods (manual or automatic) in one or more apparatuses or systems (e.g., an imaging apparatus or system, an endoscopic imaging device or system, etc.). It is also a broad object of the present disclosure to provide imaging (e.g., computed tomography (CT), Magnetic Resonance Imaging (MRI), etc.) apparatuses, systems, methods, and storage mediums for using a navigation and/or control method or methods for achieving navigation planning, autonomous navigation, and/or control through a target, sample, or object (e.g., lung airway(s) during bronchoscopy) in one or more apparatuses or systems (e.g., an imaging apparatus or system, an endoscopic imaging device or system, etc.). To address one or more of the above issues, the present disclosure provides novel supervised-autonomous driving approach(es) that integrate a novel depth-based airway tracking method(s) and a robotic bronchoscope. By simultaneously handling the tasks of advancing and centering the robot, probe, catheter, robotic bronchoscope, etc. in a target (e.g., in a lung or airway), the method(s) of the present disclosure may assist physicians in concentrating on the clinical decision-making to reach the target, which achieves or provides enhancements to the efficacy of such imaging, bronchoscopy, etc.

Additionally, one or more aspects of the present disclosure may include or relate to one or more algorithms or processes that operate to command simultaneously, or issue simultaneous commands, to: (i) bend a robot, continuum robot, robotic catheter, and/or one or more components or sections thereof; and (ii) move a stage, linear stage, and/or one or more components of or attached to a stage or linear stage. As such, one or more embodiments may simultaneously bend a robot, continuum robot, robotic catheter, one or more components or sections of a continuum robot or catheter, etc. and move a stage, linear stage, one or more components of a stage or linear stage, etc.

One or more devices, systems, methods, and storage mediums for navigation planning and/or performing control or navigation, including of a multi-section continuum robot and/or for viewing, imaging, and/or characterizing tissue and/or lesions, or an object or sample, using one or more imaging techniques (e.g., robotic bronchoscope imaging, bronchoscope imaging, etc.) or modalities (such as, but not limited to, computed tomography (CT), Magnetic Resonance Imaging (MRI), any other techniques or modalities used in imaging (e.g., Optical Coherence Tomography (OCT), Near infrared fluorescence (NIRF), Near infrared auto-fluorescence (NIRAF), Spectrally Encoded Endoscopes (SEE)), etc.) are disclosed herein. Several embodiments of the present disclosure, which may be carried out by the one or more embodiments of an apparatus, system, method, and/or computer-readable storage medium of the present disclosure are described diagrammatically and visually in the figures included herewith.

One or more embodiments of a continuum robot, apparatus, or device for performing driving/navigation and/or driving/navigation planning may include: one or more processors that operate to: detect one or more airways in an image or frame or in a view of a camera; set an airway of the one or more airways to be aimed; detect a target point in the set airway; and determine whether a predetermined insertion depth or end point is reached and whether the detected target point is within a catheter bending threshold range for a catheter of or in communication with the continuum robot such that the driving/navigation and/or the driving/navigation planning is performed by simultaneously evaluating whether to: (i) bend one or more sections of the catheter; and (ii) move a translational stage of the continuum robot or in communication with the catheter of the continuum robot. The one or more processors may further operate to one or more of the following: (i) use direct communication to reduce a latency and increase a responsiveness for the direction/navigation and/or the direction/navigation planning; (ii) operate the translational stage forwards or backwards and command bending of the one or more sections of the catheter simultaneously or separately; (iii) provide more precision over when, where, and how motion commands are applied to the catheter and/or to the translational stage; (iv) provide improved smoothness of motion and a reduction in navigation time by bending the one or more sections of the catheter and moving the translational stage simultaneously; (v) address situations where the catheter may not be able to align with the target airway by employing retracting or backwards motion of the catheter; (vi) reduce or minimize situations where control command(s) is/are sent based on an incorrect airway; (vii) identify situations which affect autonomous control, the situations including breathing motion and entering a new generation for one or more airways; and/or (viii) improve alignment timing and accuracy by sending a larger motion in a case where the target point is beyond a predetermined distance and by sending a more refined motion in a case where the target point is within or equal to a predetermined distance. The one or more processors may further operate to one or more of the following: (i) compute a direction vector from a center of a depth map to a center of the target detected airway; (ii) compute a direction vector from a center of a depth map to a center of the target detected airway where the direction vector operates to inform a virtual gamepad controller or other controller and/or the one or more processors for bending a tip of the catheter, for moving the translational stage, and/or for moving the one or more sections of the catheter; (iii) compute a direction vector from a center of a depth map to a center of the target detected airway, and advance the continuum robot in a straight line for engagement of the translational stage in a case where the direction vector has a magnitude that is less than 30% of a width of the image or frame or of the view of the camera; (iv) repeat, for each image or frame of a plurality of images or frames or for each view of the camera for a plurality of views, the determination of whether the predetermined insertion depth or end point is reached and whether the detected target point is within the catheter bending threshold range for the catheter such that the driving/navigation and/or the driving/navigation planning is performed by simultaneously evaluating whether to: (i) bend one or more sections of the catheter; and (ii) move the translational stage of the continuum robot or in communication with the catheter of the continuum robot; and/or (v) maintain a set or predetermined/calculated linear speed and a set or predetermined/calculated bending speed. The one or more processors further operate to one or more of the following: (i) in a case where the target point is within the catheter bending threshold range, set a bending speed and a magnitude for the one or more sections of the catheter and send the bend command to the one or more sections of the catheter and repeat the detection of the airways in the image or frame or in the view of the camera, the setting of the airway to be aimed, the detection of the target point in the airway, and the determinations of whether the predetermined insertion depth or end point has been reached and whether the target point is within the catheter bending threshold range; (ii) in a case where the target point is not within the catheter bending threshold range and in a case where the predetermined insertion depth or end point is reached, then determine that the target point has been reached or retract the catheter until the target point is within the catheter bending threshold range; (iii) in a case where the predetermined insertion depth or end point has not been reached, then determine whether the target point is within a stage motion threshold range for the translational stage; (iv) in a case where the predetermined insertion depth or end point has not been reached and in a case where the target point is within a stage motion threshold range for the translational stage, then send a stage motion command to the translational stage and set a stage motion direction, speed, and magnitude for the motion of the translational stage, and repeat the detection of the airways in the image or frame or in the view of the camera, the setting of the airway to be aimed, the detection of the target point in the airway, and the determinations of whether the predetermined insertion depth or end point has been reached and whether the target point is within the catheter bending threshold range; and/or (v) in a case where the predetermined insertion depth or end point has not been reached and in a case where the target point is not within a stage motion threshold range for the translational stage, then the process either waits until the target point is within the stage motion threshold range or intervention is used (e.g., manually via user input, automatically via processor intervention using stored or set data, etc.). The one or more processors may further operate to one or more of the following: (i) employ one or more command set and communication techniques using direct communication to reduce latency, increase responsiveness, and to move the translational stage and/or the one or more sections of the catheter; (ii) use a location of the target point within the image or frame or within the view of the camera to determine one or more control commands to be sent for movement of the translational stage and/or the one or more sections of the catheter; and/or (iii) use a location of the target point within the image or frame or within the view of the camera to determine one or more control commands to be sent for movement of the translational stage and/or the one or more sections of the catheter, wherein the one or more control commands includes one or more of: gamepad commands, serial commands, and/or any other form of communication/message in which the one or more processors operate to convert the communication/message to catheter movement(s) and/or translational stage movement(s), including translational stage insertion/retraction, catheter tip bending, catheter tip displacement, or other bending/displacement/state change(s) for the one or more sections of the catheter. The one or more processors may further operate to one or more of the following: (i) use command thresholding, or use command thresholding to introduce more precision over when/where/how certain motion commands are applied, improve smoothness of motion and reduce navigation time by bending the catheter and moving the translational stage simultaneously, and address situations where a catheter may not be able to align with an airway by using retraction; (ii) compute a direction vector from a center of a depth map to a center of the target detected airway where the direction vector operates to inform a virtual gamepad controller or other controller and/or the one or more processors for bending a tip of the catheter, for moving the translational stage, and/or for moving the one or more sections of the catheter; (iii) compare a magnitude to a predetermined threshold to determine a case where to command the translational stage to move where the magnitude is on a first side of the predetermined threshold and to determine another case where to command bending of the one or more sections of the catheter where the magnitude is on a second side of the predetermined threshold; (iv) compare a magnitude to a predetermined threshold to determine a case where to command the translational stage to move where the magnitude is on a first side of the predetermined threshold and to determine another case where to command bending of the one or more sections of the catheter where the magnitude is on a second side of the predetermined threshold, wherein the predetermined threshold is 30% of a width of the image or frame or of the view of the camera; (v) compare a magnitude to one or more additional predetermined thresholds upon which no commands are sent in a case where the magnitude falls on a set or predetermined side of the one or more additional predetermined thresholds; (vi) compare a magnitude to a predetermined threshold range, and use a linear or translational stage movement command or retraction command in a case where the magnitude is within the predetermined threshold range to use a retraction to bring the target point closer towards a center of the image or frame or of the view of the camera; (vii) compare a magnitude to a predetermined threshold range, and use a linear or translational stage movement command or retraction command in a case where the magnitude is within the predetermined threshold range to use a retraction to bring the target point closer towards a center of the image or frame or of the view of the camera, wherein the retraction uses a reverse Follow-The-Leader (rFTL) algorithm or process to re-orient the one or more sections of the catheter in a manner in which there is less impedance in the bending used to align the catheter with the target airway; and/or (viii) use one or more overlapping thresholds or threshold ranges, wherein insertion uses a maximum threshold of 35% of a width of the image or frame or of the view of the camera while bending has a minimum threshold of 25% of the width of the image or frame or of the view of the camera such that both the bending and the insertion are commanded in a case where the magnitude falls within 25% and 35% of the width of the image or frame or of the view of the camera and/or such that the bending and the insertion are performed using a Follow-The-Leader algorithm or process. The one or more processors may further operate to one or more of the following: (i) use target history technique(s) to reduce situations where control commands are sent based on an incorrect airway and/or to identify situations which affect autonomous control, including a breathing motion or entering a new generation of the one or more airways; (ii) repeat, for each image or frame of a plurality of images or frames or for each view of the camera for a plurality of views, the determination of whether the predetermined insertion depth or end point is reached and whether the detected target point is within the catheter bending threshold range for the catheter such that the driving/navigation and/or the driving/navigation planning is performed by simultaneously evaluating whether to: (i) bend one or more sections of the catheter; and (ii) move the translational stage of the continuum robot or in communication with the catheter of the continuum robot, with or without influence from previous one or more images or frames or from previous views of the camera; (iii) consider a difference in a location of the target airway to an unselected airway of a previous image or frame or of a previous view of the camera to determine whether a misdetection of an airway has occurred, and, in a case where the difference exceeds a predetermined amount such that a large displacement has occurred, no movement is commanded until a next image or frame or a next view of the camera; (iv) detect breathing motion in a case where a magnitude of a direction of a target airway increases beyond a predetermined range, and mute or subdue one or more control outputs or commands until the magnitude and/or the breathing motion returns to the acceptable predetermined range; (v) determine that a new branch of airway has been entered in a case where the target airway near a center of the image or frame or of the view of the camera is no longer found in a later image or frame or a later view of the camera; and/or (vi) use one or more thresholds that are one or more of: set or calculated, predetermined or fixed, adjusted manually from an input, and/or adjusted automatically based on prior changes or other one or more conditions. The one or more processors may further operate to one or more of the following: (i) use command speed and/or magnitude features or techniques to improve alignment timing and/or accuracy by sending a larger motion in a case where a target is further away than a predetermined threshold and by sending a more refined motion in a case where the target is closer than or equal to the predetermined threshold; (ii) use command speed and/or magnitude features or techniques, where the speed and/or the magnitude of one or more motion commands depend on a magnitude of a direction vector; (iii) use command speed and/or magnitude features or techniques, where command levels are proportional to a position of the target airway within a corresponding threshold, the threshold being or representing a gradient of speed and/or of magnitude of motion commands; (iv) use command speed and/or magnitude features or techniques, where command levels are proportional to a position of the target airway within a corresponding threshold, the threshold being or representing a gradient of speed and/or of magnitude of motion commands, and the gradient is discrete or continuous while a slope or range size follows a mathematical formula or a parameterized function and/or the slope or range is defined manually or automatically; (v) use command speed and/or magnitude features or techniques, where command levels are proportional to a position of the target airway within a corresponding threshold, the threshold being or representing a gradient of speed and/or of magnitude of motion commands, where the gradient has ranges within where a slope and/or size calculation different from other ranges; (vi) use any or all possible commands that controllers accept and/or use any or all possible commands that control when, how, or where each individual command is activated or deactivates; (viii) use previous state(s) for error detection and control determinations; and/or (ix) react to the target point within one or more images or frames or within one or more views of a camera by sending or commanding a predetermined or set amount of motion. In one or more embodiments, proportion can be applied to speed (a rate deltas are sent to a host), step (size of the delta), or both.

In one or more embodiments, the one or more processors may further operate to determine one or more geometry metrics, wherein the one or more geometry metrics is a depth map or maps obtained or generated by processing one or more images or frames or one or more view of the camera, and the processors may further operate to one or more of the following: advance the continuum robot or catheter to the target point; display one or more target points on a display; detect one or more objects, and fit one or more set or predetermined geometric shapes or one or more circles, rectangles, squares, ovals, octagons, and/or triangles in or on one or more detected objects; define the one or more target points based on the one or more set or predetermined geometric shapes or based on the one or more circles, rectangles, squares, ovals, octagons, and/or triangles of the one or more detected objects; set a center or portion of the circle or circles as the one or more target points for a next movement of the continuum robot; in a case where the one or more target points are not detected, then apply peak detection to the depth map or maps and use one or more detected peaks as the one or more targets; and/or in a case where one or more peaks are not detected, then use a deepest point of the depth map or maps as the one or more targets. The one or more processors may further operate to one or more of the following: (i) estimate or determine the depth map or maps using artificial intelligence (AI) architecture, where the artificial intelligence architecture includes one or more of the following: a neural network, a convolutional neural network, a generative adversarial network (GAN), a consistent generative adversarial network (cGAN), a three cycle-consistent generative adversarial network (3cGAN), recurrent neural networks, and/or any other AI architecture discussed herein; and/or (ii) use a neural network, a convolutional neural network, a generative adversarial network (GAN), a consistent generative adversarial network (cGAN), a three cycle-consistent generative adversarial network (3cGAN), recurrent neural networks, and/or any other AI architecture discussed herein to one or more of: select a target detection method or mode; use or evaluate a depth map or depth maps; perform the selected target detection method or mode; identify one or more target points; evaluate the accuracy of the identified one or more target points; and/or plan the navigation of the continuum robot, autonomously move the continuum robot to the one or more target points, or display the one or more target points on one of the one or more images, frames, or views on a display or indicate on the display or via an indicator on the display or via a light or indicator on the continuum robot that the navigation plan has been planned or determined.

In one or more embodiments, the target point(s) may be located in a lung or in an airway, and the continuum robot may be a bronchoscope. The one or more processors may further operate to perform registration or co-registration for changes due to movement, breathing, or any other change that may occur during imaging or a procedure with the continuum robot. The continuum robot may be a steerable catheter with a camera at a distal end of the steerable catheter. The continuum robot may include one or more of the following: (i) a distal bending section or portion, wherein the distal bending section or portion is commanded or instructed automatically or based on an input of a user of the continuum robot; (ii) a plurality of bending sections or portions including a distal or most distal bending portion or section and the rest of the plurality of the bending sections or portions; and/or (iii) the one or more processors further operate to instruct or command the forward motion, or the motion in the set or predetermined direction, of the translational stage and/or of the continuum robot automatically or autonomously and/or based on an input of a user of the continuum robot. The continuum robots may further include: a base and an actuator that operates to bend the plurality of the bending sections or portions independently; and the translational stage being a motorized linear stage and/or a sensor or camera that operates to move the continuum robot forward and backward, and/or in the predetermined or set direction or directions, wherein the one or more processors may operate to control the actuator and the motorized linear stage and/or the sensor or camera. The continuum robot(s) may further include a user interface of or disposed on a base, or disposed remotely from a base, the user interface operating to receive an input from a user of the continuum robot to move one or more of the plurality of bending sections or portions and/or a motorized linear stage as the translational stage and/or a sensor or camera, wherein the one or more processors further operate to receive the input from the user interface, and the one or more processors and/or the user interface operate to use a base coordinate system. The plurality of bending sections or portions may each include driving wires that operate to bend a respective section or portion of the plurality of sections or portions, wherein the driving wires may be connected to an actuator so that the actuator operates to bend one or more of the plurality of bending sections or portions using the driving wires. One or more of the following may occur: (i) the continuum robot further comprises an operational controller or joystick that operates to issue or input one or more commands or instructions as an input to the one or more processors, the input including an instruction or command to move one or more of a plurality of bending sections or portions and/or a motorized linear stage and/or a sensor or camera; (ii) the continuum robot further includes a display to display one or more images taken by the continuum robot and/or to display a navigation plan and/or an autonomous navigation path of the continuum robot; and/or (iii) the continuum robot further comprises an operational controller or joystick that operates to issue or input one or more commands or instructions to one or more processors, the input including an instruction or command to move one or more of a plurality of bending sections or portions and/or a motorized linear stage and/or a sensor or camera, and the operational controller or joystick operates to be controlled by a user of the continuum robot.

In one or more embodiments, a method for performing navigation and/or navigation planning for a continuum robot may include: detecting one or more airways in an image or frame or in a view of a camera; setting an airway of the one or more airways to be aimed; detecting a target point in the set airway; and determining whether a predetermined insertion depth or end point is reached and whether the detected target point is within a catheter bending threshold range for a catheter of or in communication with the continuum robot such that the driving/navigation and/or the driving/navigation planning is performed by simultaneously evaluating whether to: (i) bend one or more sections of the catheter; and (ii) move a translational stage of the continuum robot or in communication with the catheter of the continuum robot. One or more methods may further include any combination of continuum robot feature(s) or other method feature(s) discussed herein.

In one or more embodiments, a non-transitory computer-readable storage medium may store at least one program for causing a computer to execute a method for performing navigation and/or navigation planning for a continuum robot, the method comprising: detecting one or more airways in an image or frame or in a view of a camera; setting an airway of the one or more airways to be aimed; detecting a target point in the set airway; and determining whether a predetermined insertion depth or end point is reached and whether the detected target point is within a catheter bending threshold range for a catheter of or in communication with the continuum robot such that the driving/navigation and/or the driving/navigation planning is performed by simultaneously evaluating whether to: (i) bend one or more sections of the catheter; and (ii) move a translational stage of the continuum robot or in communication with the catheter of the continuum robot. One or more storage mediums may further include any combination of continuum robot and/or method feature(s) discussed herein.

In accordance with one or more embodiments of the present disclosure, apparatuses and systems, and methods and storage mediums for performing navigation, movement, and/or control, and/or for performing depth map-driven autonomous advancement of a multi-section continuum robot (e.g., in one or more airways, in one or more lungs, in one or more bronchoscopy pathways, etc.), may operate to characterize biological objects, such as, but not limited to, blood, mucus, lesions, tissue, etc.

Any discussion of a state, pose, position, orientation, navigation, path, or other state type discussed herein is discussed merely as a non-limiting, non-exhaustive embodiment example, and any state or states discussed herein may be used interchangeably/alternatively or additionally with the specifically mentioned type of state. Autonomous driving and/or control technique(s) may be employed to adjust, change, or control any state, pose, position, orientation, navigation, path, or other state type that may be used in one or more embodiments for a continuum robot or steerable catheter.

Physicians or other users of the apparatus or system may have reduced or saved labor and/or mental burden using the apparatus or system due to the navigation planning, autonomous navigation, control, and/or orientation (or pose, or position, etc.) feature(s) of the present disclosure. Additionally, one or more features of the present disclosure may achieve a minimized or reduced interaction with anatomy (e.g., of a patient), object, or target (e.g., tissue) during use, which may reduce the physical and/or mental burden on a patient or target. In one or more embodiments of the present disclosure, a labor of a user to control and/or navigate (e.g., rotate, translate, etc.) the imaging apparatus or system or a portion thereof (e.g., a catheter, a probe, a camera, one or more sections or portions of a catheter, probe, camera, etc.) is saved or reduced via use of the navigation planning, autonomous navigation, and/or control.

In accordance with one or more embodiments of the present disclosure, apparatuses and systems, and methods and storage mediums for performing correction(s) and/or adjustment(s) to a direction or view, and/or for performing navigation planning and/or autonomous navigation, may operate to characterize biological objects, such as, but not limited to, blood, mucus, tissue, etc.

One or more embodiments of the present disclosure may be used in clinical application(s), such as, but not limited to, intervascular imaging, intravascular imaging, bronchoscopy, atherosclerotic plaque assessment, cardiac stent evaluation, intracoronary imaging using blood clearing, balloon sinuplasty, sinus stenting, arthroscopy, ophthalmology, ear research, veterinary use and research, etc.

In accordance with at least another aspect of the present disclosure, one or more technique(s) discussed herein may be employed as or along with features to reduce the cost of at least one of manufacture and maintenance of the one or more apparatuses, devices, systems, and storage mediums by reducing or minimizing a number of optical and/or processing components and by virtue of the efficient techniques to cut down cost (e.g., physical labor, mental burden, fiscal cost, time and complexity, etc.) of use/manufacture of such apparatuses, devices, systems, and storage mediums.

The following paragraphs describe certain explanatory embodiments. Other embodiments may include alternatives, equivalents, and modifications. Additionally, the explanatory embodiments may include several novel features, and a particular feature may not be essential to some embodiments of the devices, systems, and methods that are described herein.

According to other aspects of the present disclosure, one or more additional devices, one or more systems, one or more methods, and one or more storage mediums using imaging, imaging adjustment or correction technique(s), autonomous navigation and/or planning technique(s), and/or other technique(s) are discussed herein. Further features of the present disclosure will in part be understandable and will in part be apparent from the following description and with reference to the attached drawings.

### BRIEF DESCRIPTION OF THE DRAWINGS

For the purposes of illustrating various aspects of the disclosure, wherein like numerals indicate like elements, there are shown in the drawings simplified forms that may be employed, it being understood, however, that the disclosure is not limited by or to the precise arrangements and instrumentalities shown. To assist those of ordinary skill in the relevant art in making and using the subject matter hereof, reference is made to the appended drawings and figures, wherein:

FIG. 1 illustrates at least one embodiment of an imaging, continuum robot, or endoscopic apparatus or system in accordance with one or more aspects of the present disclosure;

FIG. 2 is a schematic diagram showing at least one embodiment of an imaging, steerable catheter, or continuum robot apparatus or system in accordance with one or more aspects of the present disclosure;

FIGS. 3A-3B illustrate at least one embodiment example of a continuum robot and/or medical device that may be used with one or more technique(s), including autonomous driving/navigation and/or planning technique(s), in accordance with one or more aspects of the present disclosure;

FIGS. 3C-3D illustrate one or more principles of catheter or continuum robot tip manipulation by actuating one or more bending segments of a continuum robot or steerable catheter 104 of FIGS. 3A-3B in accordance with one or more aspects of the present disclosure;

FIG. 4 is a schematic diagram showing at least one embodiment of an imaging, continuum robot, steerable catheter, or endoscopic apparatus or system in accordance with one or more aspects of the present disclosure;

FIG. 5 is a schematic diagram showing at least one embodiment of a console or computer that may be used with one or more autonomous driving/navigation and/or planning technique(s) in accordance with one or more aspects of the present disclosure;

FIG. 6 is a flowchart of at least one embodiment of a method for planning an operation of at least one embodiment of a continuum robot or steerable catheter apparatus or system in accordance with one or more aspects of the present disclosure;

FIGS. 7A-7B are flowcharts of one or more respective embodiments of method(s) for performing navigation planning, autonomous navigation, movement detection, and/or control for a continuum robot or steerable catheter in accordance with one or more aspects of the present disclosure;

FIG. 8A shows images of at least one embodiment of an application example of navigation planning and/or autonomous navigation technique(s) and movement detection for a camera view (left), a depth map (center), and a thresholded image (right) in accordance with one or more aspects of the present disclosure;

FIG. 8B shows images of at least one embodiment of a camera view (left), a semi-transparent color coded depth map overlaid onto a camera view (center) and a thresholded image (right)) in accordance with one or more aspects of the present disclosure;

FIG. 9 shows at least one embodiment a control software or a User Interface that may be used with one or more robots, robotic catheters, robotic bronchoscopes, methods, and/or other features in accordance with one or more aspects of the present disclosure;

FIGS. 10A-10B illustrate images of at least one embodiment where the images have two airways and indicators of circle fit and target path, and FIGS. 10C-10D illustrate at least one embodiment of a bronchoscopic image with detected airways and an estimated depth map (or depth estimation) with or using detected airways, respectively, in accordance with one or more aspects of the present disclosure;

FIGS. 11A-11B illustrate at least one embodiment of a pipeline that may be used for a bronchoscope, apparatus, device, or system (or used with one or more methods or storage mediums), and a related camera view employing voice recognition, respectively, of the present disclosure in accordance with one or more aspects of the present disclosure;

FIGS. 12A-12B are respective diagrams showing two lumens and a threshold for one or more embodiments in accordance with one or more aspects of the present disclosure;

FIG. 13 is a flowchart of at least one embodiment of a method for controlling a steerable catheter in accordance with one or more aspects of the present disclosure;

FIG. 14 is a flowchart of at least one embodiment of a method for controlling a steerable catheter, including a speed setting, in accordance with one or more aspects of the present disclosure;

FIG. 15 is a flowchart of at least one embodiment of a method for controlling a steerable catheter, including setting a threshold, in accordance with one or more aspects of the present disclosure;

FIG. 16 is a flowchart of at least one embodiment of a method for controlling a steerable catheter, including blood detection, in accordance with one or more aspects of the present disclosure;

FIG. 17 is a flowchart of at least one embodiment of a method for controlling a steerable catheter, including determining whether an insertion depth or end point is reached and whether a target point is within a catheter bending threshold range, in accordance with one or more aspects of the present disclosure;

FIGS. 18A-18C illustrate a navigation screen for a clinical target location in or at a lesion reached by autonomous driving, a robotic bronchoscope in a phantom having reached the location corresponding to the location of the lesion in an ex vivo setup, and breathing cycle information using EM sensors, respectively, in accordance with one or more aspects of the present disclosure;

FIGS. 19A-19C illustrate views of at least one embodiment of a navigation algorithm performing at various branching points in a phantom where FIG. 19A shows a path on which the target location (dot) was not reached (e.g., the algorithm may not have traversed the last bifurcation where an airway on the right was not detected), where FIG. 19B shows a path on which the target location (dot) was successfully reached, and where FIG. 19C shows a path on which the target location was also successful reached in accordance with one or more aspects of the present disclosure;

FIGS. 20A-20B illustrate graphs showing success at branching point(s) with respect to Local Curvature (LC) and Plane Rotation (PR), respectively, for all data combined in one or more embodiments in accordance with one or more aspects of the present disclosure;

FIGS. 21A-21C illustrate one or more impacts of breathing motion on a performance of the one or more navigation algorithm(s) where FIG. 21A shows a path on which the target location (ex vivo #1 LLL) was reached with and without breathing motion (BM), where FIG. 21B shows a path on which the target location (ex vivo #1 RLL) was not reached without BM but was reached with BM, and where FIG. 21C shows a path on which the target location (ex vivo #1 RML) was reached without BM was not reached with BM in accordance with one or more aspects of the present disclosure;

FIGS. 22A-22B illustrate the box plots for time for the operator or the autonomous navigation/planning to bend the robotic catheter in one or more embodiments and for the maximum force for the operator or the autonomous navigation/planning at each bifurcation point in one or more embodiments in accordance with one or more aspects of the present disclosure;

FIGS. 23A-23D illustrate one or more examples of depth estimation failure and artifact robustness that may be observed in one or more embodiments in accordance with one or more aspects of the present disclosure;

FIGS. 24A-24B illustrate graphs for the dependency of the time for a bending command and the force at each bifurcation point, respectively, on the airway generation of a lung in accordance with one or more aspects of the present disclosure;

FIG. 25 shows a schematic diagram of an embodiment of a computer that may be used with one or more embodiments of an apparatus or system, or one or more methods, discussed herein in accordance with one or more aspects of the present disclosure;

FIG. 26 shows a schematic diagram of another embodiment of a computer that may be used with one or more embodiments of an imaging apparatus or system, or methods, discussed herein in accordance with one or more aspects of the present disclosure;

FIG. 27 shows a schematic diagram of at least an embodiment of a system using a computer or processor, a memory, a database, and input and output devices in accordance with one or more aspects of the present disclosure;

FIG. 28 shows a created architecture of or for a regression model(s) that may be used for navigation planning, autonomous navigation, movement detection, and/or control techniques and/or any other technique discussed herein in accordance with one or more aspects of the present disclosure;

FIG. 29 shows a convolutional neural network architecture that may be used for navigation planning, autonomous navigation, movement detection, and/or control techniques and/or any other technique discussed herein in accordance with one or more aspects of the present disclosure;

FIG. 30 shows a created architecture of or for a regression model(s) that may be used for navigation planning, autonomous navigation, movement detection, and/or control techniques and/or any other technique discussed herein in accordance with one or more aspects of the present disclosure; and

FIG. 31 is a schematic diagram of or for a segmentation model(s) that may be used for catheter connection and/or disconnection detection and/or any other technique discussed herein in accordance with one or more aspects of the present disclosure.

### DETAILED DESCRIPTION OF THE PRESENT DISCLOSURE

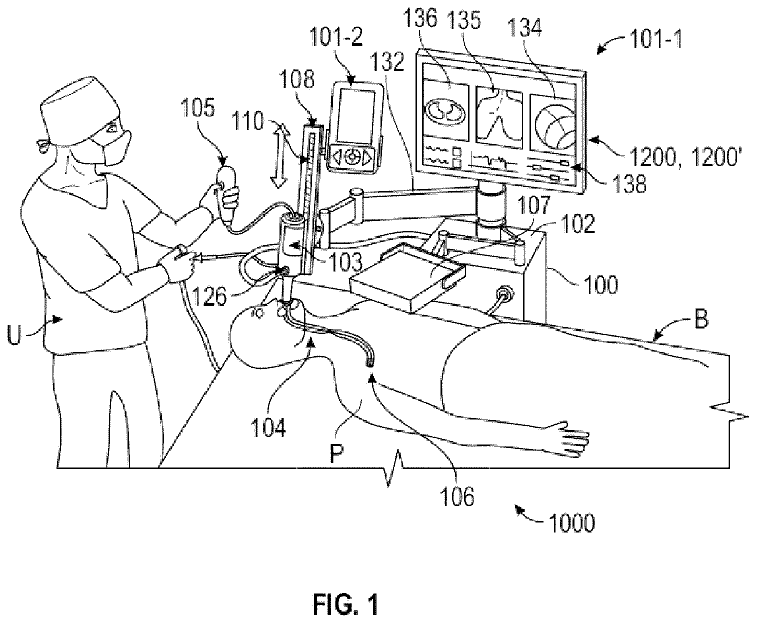

One or more devices, systems, methods and storage mediums for viewing, imaging, and/or characterizing tissue, or an object or sample, using one or more imaging techniques or modalities (such as, but not limited to, computed tomography (CT), Magnetic Resonance Imaging (MRI), any other techniques or modalities used in imaging (e.g., Optical Coherence Tomography (OCT), Near infrared fluorescence (NIRF), Near infrared auto-fluorescence (NIRAF), Spectrally Encoded Endoscopes (SEE)), etc.) are disclosed herein. Several embodiments of the present disclosure, which may be carried out by the one or more embodiments of an apparatus, system, method, and/or computer-readable storage medium of the present disclosure, are described diagrammatically and visually in FIGS. 1 through 31.

One or more embodiments of the present disclosure avoid the aforementioned issues by providing a simple and fast method or methods that provide navigation planning, autonomous driving/navigation, movement detection, and/or control technique(s) as discussed herein. In one or more embodiments of the present disclosure, navigation planning, autonomous navigation, movement detection, and/or control techniques may be performed using artificial intelligence and/or one or more processors as discussed in the present disclosure. In one or more embodiments, navigation planning, autonomous navigation, movement detection, and/or control is/are performed to reduce the amount of skill or training needed to perform imaging, medical imaging, one or more procedures (e.g., bronchoscopies), etc., and may reduce the time and cost of imaging or an overall procedure or procedures. In one or more embodiments, the navigation planning, autonomous navigation, movement detection, and/or control techniques may be used with a co-registration (e.g., CT co-registration, cone-beam CT (CBCT) co-registration, etc.) to enhance a successful targeting rate for a predetermined sample, target, or object (e.g., a lung, a portion of a lung, a vessel, a nodule, etc.) by minimizing human error. CBCT may be used to locate a target, sample, or object (e.g., the lesion(s) or nodule(s) of a lung or airways) along with an imaging device (e.g., a steerable catheter, a continuum robot, etc.) and to co-register the target, sample, or object (e.g., the lesions or nodules) with the device shown in an image to achieve proper guidance.

Accordingly, it is a broad object of the present disclosure to provide imaging (e.g., computed tomography (CT), Magnetic Resonance Imaging (MRI), etc.) apparatuses, systems, methods, and storage mediums for using a navigation and/or control method or methods (manual or automatic) in one or more apparatuses or systems (e.g., an imaging apparatus or system, an endoscopic imaging device or system, etc.). It is also a broad object of the present disclosure to provide imaging (e.g., computed tomography (CT), Magnetic Resonance Imaging (MRI), etc.) apparatuses, systems, methods, and storage mediums for using a navigation and/or control method or methods for achieving navigation planning, autonomous navigation, movement detection, and/or control through a target, sample, or object (e.g., lung airway(s) during bronchoscopy) in one or more apparatuses or systems (e.g., an imaging apparatus or system, an endoscopic imaging device or system, etc.).

By applying the target detection method(s) of the present disclosure, movement of a robot may be automatically calculated and navigation planning, autonomous navigation, and/or control to a target, sample, or object (e.g., a nodule, a lung, an airway, a predetermined location in a sample, a predetermined location in a patient, etc.) may be achieved. Additionally, by applying several levels (such as, but not limited to, three or more levels) of target detection, the advancement, movement, and/or control of the robot may be secured in one or more embodiments (e.g., the robot will not fall into a loop). In one or more embodiments, automatically calculating the movement of the robot may be provided (e.g., targeting an airway during a bronchoscopy or other lung-related procedure/imaging may be performed automatically so that any next move or control is automatic), and navigation planning and/or autonomous navigation to a predetermined target, sample, or object (e.g., a nodule, a lung, a location in a sample, a location in a patient, etc.) is feasible and may be achieved (e.g., such that a CT path does not need to be extracted, any other pre-processing may be avoided or may not need to be extracted, etc.).

The navigation planning, autonomous navigation, movement detection, and/or control may be employed so that an apparatus or system may operate to: use a depth map produced by processing one or more images obtained by or coming from a continuum robot or steerable catheter (e.g., such as, but not limited to, a bronchoscopy robotic device or system (such as, but not limited to, a robotic catheter device(s), system(s), and method(s) discussed in PCT/US2023/062508, filed on Feb. 13, 2023, which is incorporated by reference herein in its entirety)); apply thresholding using an automated method; fit a one or more set or predetermined geometric shapes or one or more circles, rectangles, squares, ovals, octagons, and/or triangles/blob in or on one or more detected objects; set a center or portion of the one or more set or predetermined geometric shapes or one or more circles, rectangles, squares, ovals, octagons, and/or triangles as a target for a next movement of the continuum robot or steerable catheter; in a case where one or more targets are not detected, then apply peak detection to the depth map and use one or more detected peaks as the one or more targets; in a case where one or more peaks are not detected, then use a deepest point of the depth map as the one or more targets; and/or automatically advancing the continuum robot or steerable catheter to the detected one or more targets or choose automatically, semi-automatically, or manually one of the detected one or more targets. In one or more embodiments where a target position is outside an acceptable or predetermined area, then the method may further include returning to the depth map generation step. In one or more embodiments, automatic targeting the movement, navigation, and/or control may be provided (e.g., in a lung application, airway targeting which plans the next move may be automatic). In one or more embodiments, the one or more processors may apply thresholding to define an area of a target, sample, or object (e.g., to define an area of an airway/vessel or other target). In one or more embodiments, fitting the one or more set or predetermined geometric shapes or one or more circles, rectangles, squares, ovals, octagons, and/or triangles in the one or more detected objects may include blob detection and/or peak detection to identify the one or more targets and/or to confirm the identified or detected one or more targets. The one or more processors may further operate to: take a still image or images, use or process a depth map for the taken still image or images, apply thresholding to the taken still image or images and detect one or more objects, fit a one or more set or predetermined geometric shapes or one or more circles, rectangles, squares, ovals, octagons, and/or triangles/blob for the one or more objects of the taken still image or images, define one or more targets for a next movement of the continuum robot or steerable catheter based on the taken still image or images; and advance the continuum robot or steerable catheter to the one or more targets or choose automatically, semi-automatically, or manually one of the detected one or more targets. The one or more processors further operate to repeat any of the features (such as, but not limited to, obtaining a depth map, performing thresholding, performing a fit based on one or more set or predetermined geometric shapes or based on one or more circles, rectangles, squares, ovals, octagons, and/or triangles, performing peak detection, determining a deepest point, etc.) of the present disclosure for a next or subsequent image or images. Such next or subsequent images may be evaluated to distinguish from where to register the continuum robot or steerable catheter with an external image, and/or such next or subsequent images may be evaluated to perform registration or co-registration for changes due to movement, breathing, or any other change that may occur during imaging or a procedure with a continuum robot or steerable catheter. In one or more embodiments, a navigation plan may include (and may not be limited to) one or more of the following: a next movement of the continuum robot, one or more next movements of the continuum robot, one or more targets, all of the next movements of the continuum robot, all of the determined next movements of the continuum robot, one or more next movements of the continuum robot to reach the one or more targets, etc. In one or more embodiments, the navigation plan may be updated or data may be added to the navigation plan, where the data may include any additionally determined next movement of the continuum robot.

Any discussion of a state, pose, position, orientation, navigation, path, or other state type discussed herein is discussed merely as a non-limiting, non-exhaustive embodiment example, and any state or states discussed herein may be used interchangeably/alternatively or additionally with the specifically mentioned type of state. Autonomous driving and/or control technique(s) may be employed to adjust, change, or control any state, pose, position, orientation, navigation, path, or other state type that may be used in one or more embodiments for a continuum robot or steerable catheter.

Physicians or other users of the apparatus or system may have reduced or saved labor and/or mental burden using the apparatus or system due to the navigation planning, autonomous navigation, control, and/or orientation (or pose, or position, etc.) feature(s) of the present disclosure. Additionally, one or more features of the present disclosure may achieve a minimized or reduced interaction with anatomy (e.g., of a patient), object, or target (e.g., tissue) during use, which may reduce the physical and/or mental burden on a patient or target. In one or more embodiments of the present disclosure, a labor of a user to control and/or navigate (e.g., rotate, translate, etc.) the imaging apparatus or system or a portion thereof (e.g., a catheter, a probe, a camera, one or more sections or portions of a catheter, probe, camera, etc.) is saved or reduced via use of the navigation planning, autonomous navigation, and/or control.

An imaging device or system, or a portion of the imaging device or system (e.g., a catheter, a probe, etc.), the continuum robot, and/or the steerable catheter may include multiple sections or portions, and the multiple sections or portions may be multiple bending sections or portions. The imaging device or system may include manual and/or automatic navigation and/or control features. For example, a user of the imaging device or system (or steerable catheter, continuum robot, etc.) may control each section or portion, and/or the imaging device or system (or steerable catheter, continuum robot, etc.) may operate to automatically control (e.g., robotically control) each section or portion, such as, but not limited to, via one or more navigation planning, autonomous navigation, movement detection, and/or control techniques.

Navigation, control, and/or orientation feature(s) may include, but are not limited to, implementing mapping of a pose (angle value(s), plane value(s), etc.) of a first portion or section (e.g., a tip portion or section, a distal portion or section, a predetermined or set portion or section, a user selected or defined portion or section, etc.) to a stage position/state (or a position/state of another structure being used to map path or path-like information), controlling angular position(s) of one or more of the multiple portions or sections, controlling rotational orientation or position(s) of one or more of the multiple portions or sections, controlling (manually or automatically (e.g., robotically)) one or more other portions or sections of the imaging device or system (e.g., continuum robot, steerable catheter, etc.) to match the navigation/orientation/position/pose of the first portion or section in a case where the one or more other portions or sections reach (e.g., subsequently reach, reach at a different time, etc.) the same position (e.g., in a target, in an object, in a sample, in a patient, in a frame or image, etc.) during navigation in or along a first direction of a path of the imaging device or system, controlling each of the sections or portions of the imaging device or system to retrace and match prior respective position(s) of the sections or portions in a case where the imaging device or system is moving or navigated in a second direction (e.g., in an opposite direction along the path, in a return direction along the path, in a retraction direction along the path, etc.) along the path, etc. For example, an imaging device or system (or portion thereof, such as, but not limited to, a probe, a catheter, a camera, etc.) may enter a target along a path where a first section or portion of the imaging device or system (or portion of the device or system) is used to set the navigation or control path and position(s), and each subsequent section or portion of the imaging device or system (or portion of the device or system) is controlled to follow the first section or portion such that each subsequent section or portion matches the orientation and position of the first section or portion at each location along the path. During retraction, each section or portion of the imaging device or system is controlled to match the prior orientation and position (for each section or portion) for each of the locations along the path. As such, an imaging device or system (or catheter, probe, camera, etc. of the device or system) may enter and exit a target, an object, a specimen, a patient (e.g., a lung of a patient, an esophagus of a patient, another portion of a patient, another organ of a patient, a vessel of a patient, etc.), etc. along the same path and using the same orientation for entrance and exit to achieve an optimal navigation, orientation, and/or control path. The navigation, control, and/or orientation feature(s) are not limited thereto, and one or more devices or systems of the present disclosure may include any other desired navigation, control, and/or orientation specifications or details as desired for a given application or use. In one or more embodiments and while not limited thereto, the first portion or section may be a distal or tip portion or section of the imaging device or system. In one or more embodiments, the first portion or section may be any predetermined or set portion or section of the imaging device or system, and the first portion or section may be predetermined or set manually by a user of the imaging device or system or may be set automatically by the imaging device or system.

In one or more embodiments of the present disclosure (and while not limited to only this definition), a “change of orientation” may be defined in terms of direction and magnitude. For example, each interpolated step may have a same direction, and each interpolated step may have a larger magnitude as each step approaches a final orientation. Due to kinematics of one or more embodiments, any motion along a single direction may be the accumulation of a small motion in that direction. The small motion may have a unique or predetermined set of wire position changes to achieve the orientation change. Large or larger motion(s) in that direction may use a plurality of the small motions to achieve the large or larger motion(s). Dividing a large change into a series of multiple changes of the small or predetermined/set change may be used as one way to perform interpolation. Interpolation may be used in one or more embodiments to produce a desired or target motion, and at least one way to produce the desired or target motion may be to interpolate the change of wire positions.

Any of the features of the present disclosure may be used with artificial intelligence (AI) feature(s), including the AI features discussed herein. Using artificial intelligence, for example (but not limited to), deep/machine learning, residual learning, a computer vision task (keypoint or object detection and/or image segmentation), using a unique architecture structure of a model or models, using a unique training process, using input data preparation techniques, using input mapping to the model, using post-processing and interpretation of the output data, etc., one or more embodiments of the present disclosure may achieve a better or maximum success rate of navigation planning, autonomous navigation, movement detection, and/or control without (or with less) user interactions, and may reduce processing time to perform navigation planning, autonomous navigation, movement detection, and/or control techniques. One or more artificial intelligence structures may be used to perform navigation planning, autonomous navigation, movement detection, and/or control techniques, such as, but not limited to, a neural net or network (e.g., the same neural net or network that operates to determine whether a video or image from an endoscope or other imaging device is working; the same neural net or network that has obtained or determined the depth map, etc.; an additional neural net or network, a convolutional network (e.g., a convolutional neural network (CNN) may operate to use a visual output of a camera (e.g., a bronchoscopic camera, a catheter camera, a detector, etc.) to automatically detect one or more airways, one or more objects, one or more areas of one or more airways or objects, etc.), recurrent network, another network discussed herein, etc.). In one or more embodiments, an apparatus for performing navigation planning, autonomous navigation, movement detection, and/or control using artificial intelligence may include: a memory; and one or more processors in communication with the memory, the one or more processors operating to: using or processing a depth map produced by processing one or more images obtained by or coming from a continuum robot or steerable catheter (e.g., such as, but not limited to, a bronchoscopy robotic device or system (such as, but not limited to, a robotic catheter device(s), system(s), and method(s) discussed in PCT/US2023/062508, filed on Feb. 13, 2023, which is incorporated by reference herein in its entirety), obtained by a continuum robot or steerable catheter from the memory, obtained via one or more neural networks, etc.); applying thresholding using an automated method and detecting one or more objects; fitting one or more set or predetermined geometric shapes or one or more circles, rectangles, squares, ovals, octagons, and/or triangles in or on the one or more detected objects; defining one or more targets for a next movement of the continuum robot or steerable catheter; and advancing the continuum robot or steerable catheter to the one or more targets or choose automatically, semi-automatically, or manually one of the detected one or more targets. In one or more embodiments, defining the one or more targets may include setting a center or portion(s) of the one or more set or predetermined geometric shapes or the one or more circles, rectangles, squares, ovals, octagons, and/or triangles as one or more targets for a next movement of the continuum robot or steerable catheter. In one or more embodiments, the method may further include the following steps: in a case where the one or more targets are not detected, then applying peak detection to the depth map and using one or more detected peaks as the one or more targets; and/or in a case where one or more peaks are not detected, then using a deepest point of the depth map as the one or more targets. In one or more embodiments, the continuum robot or steerable catheter may be automatically advanced during the advancing step. In one or more embodiments, automatic targeting the movement, navigation, and/or control may be provided (e.g., in a lung application, airway targeting which plans the next move may be automatic). One or more of the artificial intelligence features discussed herein that may be used in one or more embodiments of the present disclosure, includes but is not limited to, using one or more of deep learning, a computer vision task, keypoint detection, a unique architecture of a model or models, a unique training process or algorithm, a unique optimization process or algorithm, input data preparation techniques, input mapping to the model, pre-processing, post-processing, and/or interpretation of the output data as substantially described herein or as shown in any one of the accompanying drawings. Neural networks may include a computer system or systems. In one or more embodiments, a neural network may include or may comprise an input layer, one or more hidden layers of neurons or nodes, and an output layer. The input layer may be where the values are passed to the rest of the model. While not limited thereto, in one or more continuum robot or steerable catheter application(s), the input layer may be the place where the transformed navigation, movement, and/or control data may be passed to a model for evaluation. In one or more embodiments, the hidden layer(s) may be a series of layers that contain or include neurons or nodes that establish connections between the neurons or nodes in the other hidden layers. Through training, the values of each of the connections may be altered so that, due to the training, the system/systems will trigger when the expected pattern is detected. The output layer provides the result(s) of the model. In the case of the continuum robot or steerable catheter navigation planning, autonomous navigation, movement detection, and/or control application(s), this may be a Boolean (true/false) value for detecting the one or more targets, for detecting the one or more objects, detecting the one or more peaks, detecting the deepest point, or any other calculation, detection, or process/technique discussed herein. One or more features discussed herein may be determined using a convolutional auto-encoder, Gaussian filters, Haralick features, and/or thickness or shape of the sample(s), target(s), or object(s). In one or more embodiments, and while not limited thereto, residual network(s), such as deep residual network(s), wide residual network(s), aggregated residual transformation network(s), other types of residual network(s) (e.g., ResNet, ResNeXt, etc.), any combination thereof, etc., may be used to perform AI feature(s).

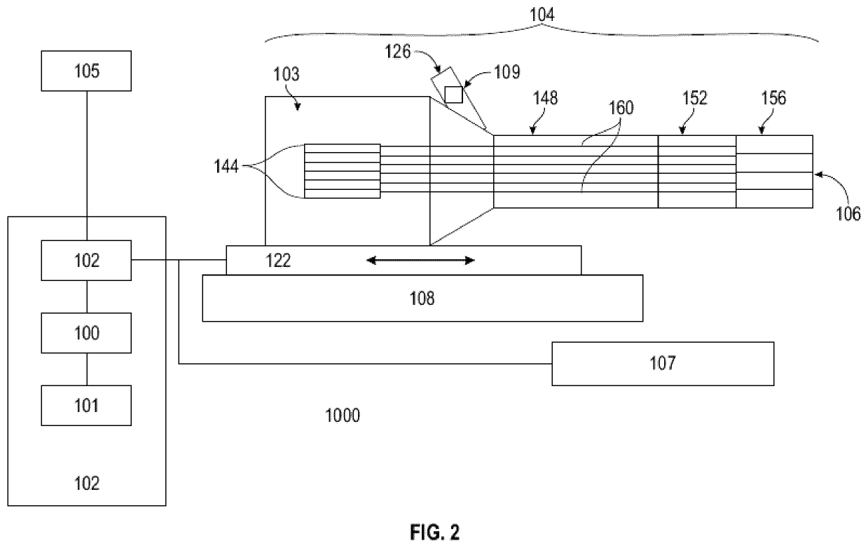

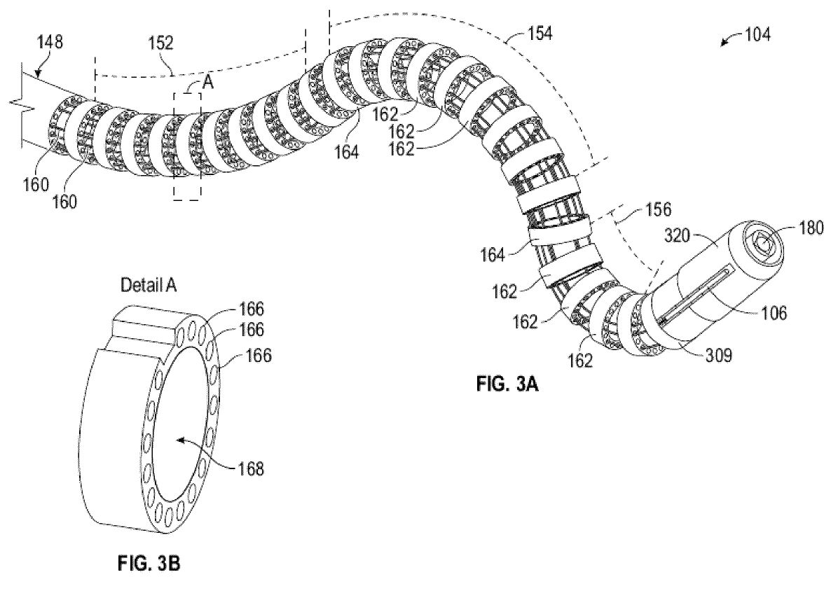

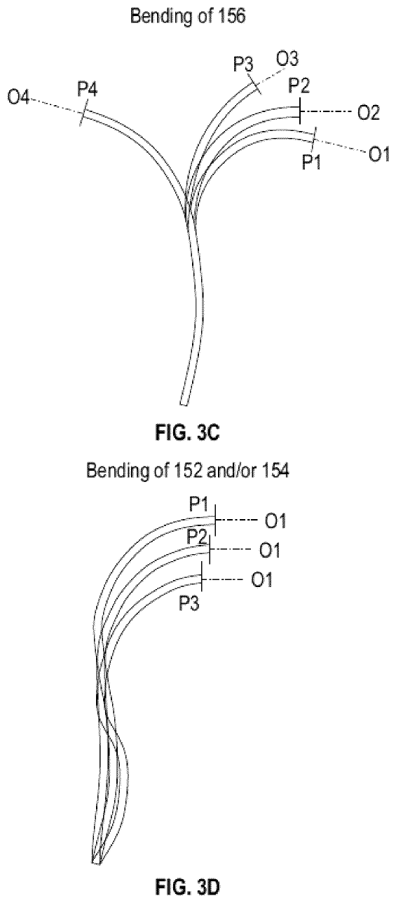

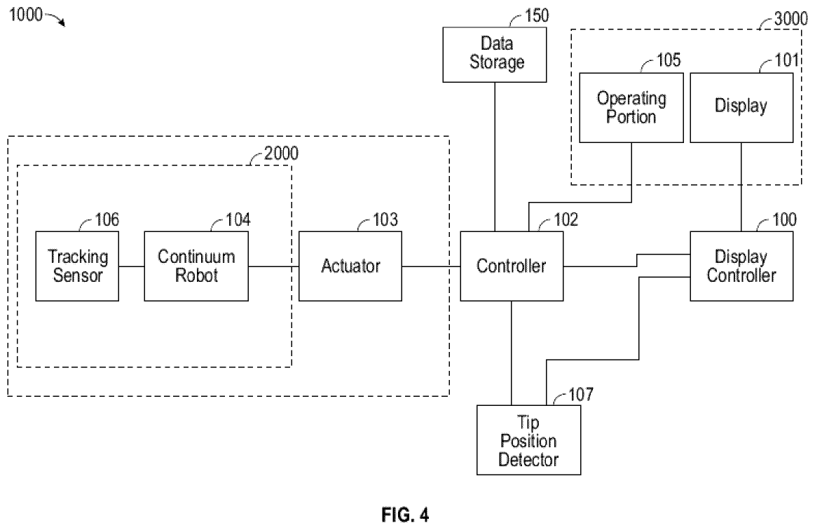

FIG. 1 illustrates a simplified representation of a medical environment, such as an operating room, where a robotic catheter system 1000 may be used. FIG. 2 illustrates a functional block diagram that may be used in at least one embodiment of the robotic catheter system 1000. FIGS. 3A-3D represent at least one embodiment of the catheter 104 (see FIGS. 3A-3B) and bending for the catheter 104 (as shown in FIGS. 3C-3D). FIG. 4 illustrates a logical block diagram that may be used for the robotic catheter system 1000. In at least this embodiment example, the system 1000 may include a computer cart (see e.g., the controller 100, 102 in FIG. 1) operatively connected to a steerable catheter or continuum robot 104 via a robotic platform 108. The robotic platform 108 includes one or more than one robotic arm 132 and a rail 110 (see e.g., FIGS. 1-2) and/or linear translation stage 122 (see e.g., FIG. 2).

As shown in FIGS. 1-4 of the present disclosure, one or more embodiments of a system 1000 for performing navigation planning, autonomous navigation, movement detection, and/or control (e.g., for a continuum robot, a steerable catheter, etc.) may include one or more of the following: a display controller 100, a display 101-1, a display 101-2, a controller 102, an actuator 103, a continuum device (also referred to herein as a “steerable catheter” or “an imaging device”) 104, an operating portion 105, a camera or tracking sensor 106 (e.g., an electromagnetic (EM) tracking sensor), a catheter tip position/orientation/pose/state detector 107 (which may be optional (e.g., while a camera 106 may be used without an EM tracking or tracking sensor in one or more embodiments, the tracking or EM tracking sensor may be used along with the camera 106 in one or more embodiments; the position/state detector 107 may be used in one or more embodiments, but the position/state detector 107 may be optional in one or more embodiments such that the camera 106 may be used without a position/state detector 107 in one or more embodiments), and a rail 110 (which may be attached to or combined with a linear translation stage 122) (for example, as shown in at least FIGS. 1-2). The system 1000 may include one or more processors, such as, but not limited to, a display controller 100, a controller 102, a CPU 120, a controller 50, a CPU 51, a console or computer 1200 or 1200′, a CPU 1201, any other processor or processors discussed herein, etc., that operate to execute a software program, to control the one or more adjustment, control, and/or smoothing technique(s) discussed herein, and to control display of a navigation screen on one or more displays 101. The one or more processors (e.g., the display controller 100, the controller 102, the CPU 120, the controller 50, the CPU 51, the console or computer 1200 or 1200′, the CPU 1201, any other processor or processors discussed herein, etc.) may generate a three dimensional (3D) model of a structure (for example, a branching structure like airway of lungs of a patient, an object to be imaged, tissue to be imaged, etc.) based on images, such as, but not limited to, CT images, MRI images, etc. Alternatively, the 3D model may be received by the one or more processors (e.g., the display controller 100, the controller 102, the CPU 120, the controller 50, the CPU 51, the console or computer 1200 or 1200′, the CPU 1201, any other processor or processors discussed herein, etc.) from another device. A two-dimensional (2D) model may be used instead of 3D model in one or more embodiments. The 2D or 3D model may be generated before a navigation starts. Alternatively, the 2D or 3D model may be generated in real-time (in parallel with the navigation). In the one or more embodiments discussed herein, examples of generating a model of branching structure are explained. However, the models may not be limited to a model of branching structure. For example, a model of a route direct to a target may be used instead of the branching structure. Alternatively, a model of a broad space may be used, and the model may be a model of a place or a space where an observation or a work is performed by using a continuum robot 104 explained below.

In FIG. 1, a user U (e.g., a physician, a technician, etc.) may control the robotic catheter system 1000 via a user interface unit (operation unit) to perform an intraluminal procedure on a patient P positioned on an operating table B. The user interface may include at least one of a main or first display 101-1 (a first user interface unit), a second display 101-2 (a second user interface unit), and a handheld controller 105 (a third user interface unit). The main or first display 101-1 may include, for example, a large display screen attached to the system 1000 and/or the controllers 101, 102 of the system 1000 or mounted on a wall of the operating room and may be, for example, designed as part of the robotic catheter system 1000 or may be part of the operating room equipment. Optionally, there may be a secondary display 101-2 that is a compact (portable) display device configured to be removably attached to the robotic platform 108. Examples of the second or secondary display 101-2 may include, but are not limited to, a portable tablet computer, a mobile communication device (a cellphone), a tablet, a laptop, etc.

The steerable catheter 104 may be actuated via an actuator unit 103. The actuator unit 103 may be removably attached to the robotic platform 108 or any component thereof (e.g., the robotic arm 132, the rail 110, and/or the linear translation stage 122). The handheld controller 105 may include a gamepad-like controller with a joystick having shift levers and/or push buttons, and the controller 105 may be a one-handed controller or a two-handed controller. In one embodiment, the actuator unit 103 may be enclosed in a housing having a shape of a catheter handle. One or more access ports 126 may be provided in or around the catheter handle. The access port 126 may be used for inserting and/or withdrawing end effector tools and/or fluids when performing an interventional procedure of the patient P.

In one or more embodiments, the system 1000 includes at least a system controller 102, a display controller 100, and the main display 101-1. The main display 101-1 may include a conventional display device such as a liquid crystal display (LCD), an OLED display, a QLED display, etc. The main display 101-1 may provide or display a graphic interface unit (GUI) configured to display one or more views. These views may include a live view image 134, an intraoperative image 135, a preoperative image 136, and other procedural information 138. Other views that may be displayed include a model view, a navigational information view, and/or a composite view. The live image view 134 may be an image from a camera at the tip of the catheter 104. The live image view 134 may also include, for example, information about the perception and navigation of the catheter 104. The preoperative image 136 may include pre-acquired 3D or 2D medical images of the patient P acquired by conventional imaging modalities such as computer tomography (CT), magnetic resonance imaging (MRI), ultrasound imaging, or any other desired imaging modality. The intraoperative image 135 may include images used for image guided procedure such images may be acquired by fluoroscopy or CT imaging modalities (or another desired imaging modality). The intraoperative image 135 may be augmented, combined, or correlated with information obtained from a sensor, camera image, or catheter data.

In the various embodiments where a catheter tip tracking sensor 106 is used in addition to the camera 106, the sensor may be located at the distal end of the catheter 104. The catheter tip tracking sensor 106 may be, for example, an electromagnetic (EM) sensor. If an EM sensor is used, a catheter tip position detector 107 may be included in the robotic catheter system 1000; the catheter tip position detector 107 may include an EM field generator operatively connected to the system controller 102. As aforementioned, the camera 106 may be used alone or in combination with the tracking sensor 106 and the position detector 107 to determine and output detected positional/state information to the system controller 102. Suitable electromagnetic sensors for use with a steerable catheter may be used with any feature of the present disclosure, including the sensors discussed, for example, in U.S. Pat. No. 6,201,387 and in International Pat. Pub. WO 2020/194212 A1, which are incorporated by reference herein in their entireties.

While not limited to such a configuration, the display controller 100 may acquire position/orientation/navigation/pose/state (or other state) information of the continuum robot 104 from a controller 102. Alternatively, the display controller 100 may acquire the position/orientation/navigation/pose/state (or other state) information directly from the camera 106 and/or from a tip position/orientation/navigation/pose/state (or other state) detector 107. As aforementioned, the camera 106 may be used alone or in combination with the tracking sensor 106 and the position detector 107 to determine and output detected positional/state information to the system controller 102. The continuum robot 104 may be a catheter device (e.g., a steerable catheter or probe device). The continuum robot 104 may be attachable/detachable to the actuator 103, and the continuum robot 104 may be disposable.

Similar to FIG. 1, FIG. 2 illustrates the robotic catheter system 1000 including the system controller 102 operatively connected to the display controller 100, which is connected to the first display 101-1 and to the second display 101-2. The system controller 102 is also connected to the actuator 103 via the robotic platform 108 or any component thereof (e.g., the robotic arm 132, the rail 110, and/or the linear translation stage 122). The actuator unit 103 may include a plurality of motors 144 that operate to control a plurality of drive wires 160 (while not limited to any particular number of drive wires 160, FIG. 2 shows that six (6) drive wires 160 are being used in the subject embodiment example). The drive wires 160 travel through the steerable catheter or continuum robot 104. One or more access ports 126 may be located on the catheter 104 (and may include an insertion/extraction detector 109). The catheter 104 may include a proximal section 148 located between the actuator 103 and the proximal bending section 152, where the drive wires 160 operate to actuate the proximal bending section 152. Three of the six drive wires 160 continue through the distal bending section 156 where the drive wires 160 operate to actuate the distal bending section 156 and allow for a range of movement. FIG. 2 is shown with two bendable sections 152, 156. Other embodiments as described herein may have three bendable sections (see e.g., FIGS. 3A-3D). A single bending section may be provided, or alternatively, four or more bendable sections may be present in one or more embodiments of the catheter 104.

FIGS. 3A-3B show continuum robot 104 embodiment(s) that may be used in system 1000 or other system discussed herein. FIG. 3A shows embodiment(s) of a steerable catheter 104. The steerable catheter 104 may have a non-steerable proximal section 148, a steerable distal section 156, and a catheter tip 320. The proximal section 148 and distal bendable section 156 (including portions 152, 154, and 156 in FIG. 3A) are joined to each other by a plurality of drive wires 160 arranged along the wall of the catheter 104. The proximal section 148 is configured with through-holes (or thru-holes) or grooves or conduits to pass drive wires 160 from the distal section 152, 154, 156 to the actuator unit 103. The distal section 152, 154, 156 is comprised of a plurality of bending segments including at least a distal segment 156, a middle segment 154, and a proximal segment 152. Each bending segment is bent by actuation of at least some of the plurality of drive wires 160 (driving members). The posture of the catheter 104 may be supported by supporting wires (support members) also arranged along the wall of the catheter 104 (as discussed in U.S. Pat. Pub. US2021/0308423, which is incorporated by reference herein in its entirety). The proximal ends of drive wires 160 are connected to individual actuators or motors 144 of the actuator unit 103, while the distal ends of the drive wires 160 are selectively anchored to anchor members in the different bending segments of the distal bendable section 152, 154, 156.

Each bending segment is formed by a plurality of ring-shaped components (rings) with through-holes (or thru-holes), grooves, or conduits along the wall of the rings. The ring-shaped components are defined as wire-guiding members 162 or anchor members 164 depending on a respective function(s) within the catheter 104. The anchor members 164 are ring-shaped components onto which the distal end of one or more drive wires 160 are attached in one or more embodiments. The wire-guiding members 162 are ring-shaped components through which some drive wires 160 slide through (without being attached thereto).

As shown in FIG. 3B, detail “A” obtained from the identified portion of FIG. 3A illustrates at least one embodiment of a ring-shaped component (a wire-guiding member 162 or an anchor member 164). Each ring-shaped component 162, 164 may include a central opening which may form a tool channel 168 and may include a plurality of conduits 166 (grooves, sub-channels, or through-holes (or thru-holes)) arranged lengthwise (and which may be equidistant from the central opening) along the annular wall of each ring-shaped component 162, 164. Inside the ring-shaped component(s) 162, 164, an inner cover, such as is described in U.S. Pat. Pub. US2021/0369085 and US2022/0126060, which are incorporated by reference herein in their entireties, may be included to provide a smooth inner channel and to provide protection. The non-steerable proximal section 148 may be a flexible tubular shaft and may be made of extruded polymer material. The tubular shaft of the proximal section 148 also may have a central opening or tool channel 168 and plural conduits 166 along the wall of the shaft surrounding the tool channel 168. An outer sheath may cover the tubular shaft and the steerable section 152, 154, 156. In this manner, at least one tool channel 168 formed inside the steerable catheter 104 provides passage for an imaging device and/or end effector tools from the insertion port 126 to the distal end of the steerable catheter 104.

The actuator unit 103 may include, in one or more embodiments, one or more servo motors or piezoelectric actuators. The actuator unit 103 may operate to bend one or more of the bending segments of the catheter 104 by applying a pushing and/or pulling force to the drive wires 160.

As shown in FIG. 3A, each of the three bendable segments of the steerable catheter 104 has a plurality of drive wires 160. If each bendable segment is actuated by three drive wires 160, the steerable catheter 104 has nine driving wires arranged along the wall of the catheter 104. Each bendable segment of the catheter 104 is bent by the actuator unit 103 by pushing or pulling at least one of these nine drive wires 160. Force is applied to each individual drive wire in order to manipulate/steer the catheter 104 to a desired pose. The actuator unit 103 assembled with steerable catheter 104 may be mounted on the robotic platform 108 or any component thereof (e.g., the robotic arm 132, the rail 110, and/or the linear translation stage 122). The robotic platform 108, the rail 110, and/or the linear translation stage 122 may include a slider and a linear motor. In other words, the robotic platform 108 or any component thereof (e.g., the robotic arm 132, the rail 110, and/or the linear translation stage 122) is motorized, and may be controlled by the system controller 102 to insert and remove the steerable catheter 104 to/from the target, sample, or object (e.g., the patient, the patient's bodily lumen, one or more airways, a lung, etc.).

An imaging device 180 that may be inserted through the tool channel 168 includes an endoscope camera (videoscope) along with illumination optics (e.g., optical fibers or LEDs) (or any other camera or imaging device, tool, etc. discussed herein or known to those skilled in the art). The illumination optics provide light to irradiate the lumen and/or a lesion target which is a region of interest within the target, sample, or object (e.g., in a patient). End effector tools may refer to endoscopic surgical tools including clamps, graspers, scissors, staplers, ablation or biopsy needles, and other similar tools, which serve to manipulate body parts (organs or tumorous tissue) during imaging, examination, or surgery. The imaging device 180 may be what is commonly known as a chip-on-tip camera and may be color (e.g., take one or more color images) or black-and-white (e.g., take one or more black-and-white images). In one or more embodiments, a camera may support color and black-and-white images.

In some embodiments, the camera alone or in combination with a tracking sensor (e.g., an EM tracking sensor) 106 may be attached to the catheter tip 320. In an embodiment where an EM tracking sensor 106 is used along with the camera 106, the steerable catheter 104 and the tracking sensor 106 may be tracked by the tip position detector 107. Specifically, the tip position detector 107 detects a position of the tracking sensor 106, and outputs the detected positional information to the system controller 102. Again, the camera 106 may be used alone or in combination with the tracking sensor 106 and the position detector 107 to determine and output detected positional/state information to the system controller 102. The system controller 102, receives the positional information from the tip position detector 107, and continuously records and displays the position of the steerable catheter 104 with respect to the coordinate system of the target, sample, or object (e.g., a patient, a lung, an airway(s), a vessel, etc.). The system controller 102 operates to control the actuator unit 103 and the robotic platform 108 or any component thereof (e.g., the robotic arm 132, the rail 110, and/or the linear translation stage 122) in accordance with the manipulation commands input by the user U via one or more of the input and/or display devices (e.g., the handheld controller 105, a GUI at the main display 101-1, touchscreen buttons at the secondary display 101-2, etc.).

FIG. 3C and FIG. 3D show exemplary catheter tip manipulations by actuating one or more bending segments of the steerable catheter 104. As illustrated in FIG. 3C, manipulating only the most distal segment 156 of the steerable section may change the position and orientation of the catheter tip 320. On the other hand, manipulating one or more bending segments (152 or 154) other than the most distal segment may affect only the position of catheter tip 320, but may not affect the orientation of the catheter tip 320. In FIG. 3C, actuation of distal segment 156 changes the catheter tip from a position P1 having orientation O1, to a position P2 having orientation O2, to position P3 having orientation O3, to position P4 having orientation O4, etc. In FIG. 3D, actuation of the middle segment 152 and/or the middle segment 154 may change the position of the catheter tip 320 from a position P1 having orientation O1 to a position P2 and position P3 having the same orientation O1. Here, it should be appreciated by those skilled in the art that exemplary catheter tip manipulations shown in FIG. 3C and FIG. 3D may be performed during catheter navigation (e.g., while inserting the catheter 104 through tortuous anatomies, one or more targets, samples, objects, a patient, etc.). In the present disclosure, the one or more catheter tip manipulations shown in FIG. 3C and FIG. 3D may apply namely to the targeting mode applied after the catheter tip 320 has been navigated to a predetermined distance (a targeting distance) from the target, sample, or object.

The actuator 103 may proceed or retreat along a rail 110 (e.g., to translate the actuator 103, the continuum robot/catheter 104, etc.), and the actuator 103 and continuum robot 104 may proceed or retreat in and out of the patient's body or other target, object, or specimen (e.g., tissue). As shown in FIG. 3B, the catheter device 104 may include a plurality of driving backbones and may include a plurality of passive sliding backbones. In one or more embodiments, the catheter device 104 may include at least nine (9) driving backbones and at least six (6) passive sliding backbones. The catheter device 104 may include an atraumatic tip at the end of the distal section of the catheter device 104.

FIG. 4 illustrates that a system 1000 may include the system controller 102 which may operate to execute software programs and control the display controller 100 to display a navigation screen (e.g., a live view image 134) on the main display 101-1 and/or the secondary display 101-2. The display controller 100 may include a graphics processing unit (GPU) or a video display controller (VDC) (or any other suitable hardware discussed herein or known to those skilled in the art.

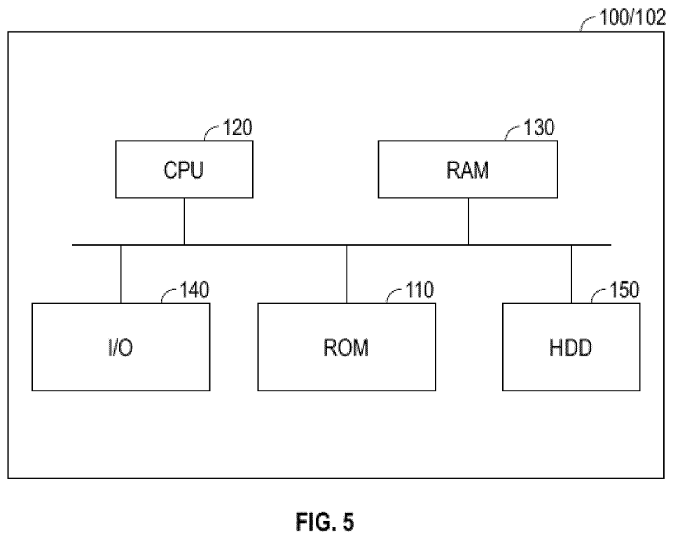

FIG. 5 illustrates components of the system controller 102 and/or the display controller 100. The system controller 102 and the display controller 100 may be configured separately. Alternatively, the system controller 102 and the display controller 100 may be configured as one device. In either case, the system controller 102 and the display controller 100 may include substantially the same components in one or more embodiments. For example, as shown in FIG. 5, the system controller 102 and the display controller 100 may include a central processing unit (CPU 120) (which may be comprised of one or more processors (microprocessors)), a random access memory (RAM 130) module, an input/output (I/O 140) interface, a read only memory (ROM 110), and data storage memory (e.g., a hard disk drive (HDD 150) or solid state drive (SSD)).

The ROM 110 and/or HDD 150 store the operating system (OS) software, and software programs necessary for executing the functions of the robotic catheter system 1000 as a whole. The RAM 130 is used as a workspace memory. The CPU 120 executes the software programs developed in the RAM 130. The I/O 140 inputs, for example, positional information to the display controller 102, and outputs information for displaying the navigation screen to the one or more displays (main display 101-1 and/or secondary display 101-2). In the embodiments descried below, the navigation screen is a graphical user interface (GUI) generated by a software program but, it may also be generated by firmware, or a combination of software and firmware.

The system controller 102 may control the steerable catheter 104 based on any known kinematic algorithms applicable to continuum or snake-like catheter robots. For example, the system controller controls the steerable catheter 104 based on an algorithm known as follow the leader (FTL) algorithm. With the FTL algorithm, the most distal segment 156 is actively controlled with forward kinematic values, while the middle segment 154 and the other middle or proximal segment 152 (following sections) of the steerable catheter 104 move at a first position in the same way as the distal section moved at the first position or a second position near the first position.

The display controller 100 may acquire position information of the steerable catheter 104 from system controller 102. Alternatively, the display controller 100 may acquire the position information directly from the tip position detector 107. The steerable catheter 104 may be a single-use or limited-use catheter device. In other words, the steerable catheter 104 may be attachable to, and detachable from, the actuator unit 103 to be disposable.

During a procedure, the display controller 100 may generate and output a live-view image or a navigation screen to the main display 101-1 and/or the secondary display 101-2 based on the 3D model of a target, sample, or object (e.g., a lung, an airway, a vessel, a patient's anatomy (a branching structure), etc.) and the position information of at least a portion of the catheter (e.g., position of the catheter tip 320) by executing pre-programmed software routines. The navigation screen may indicate a current position of at least the catheter tip 320 on the 3D model. By observing the navigation screen, a user may recognize the current position of the steerable catheter 104 in the branching structure. Upon completing navigation to a desired target, one or more end effector tools may be inserted through the access port 126 at the proximal end of the catheter 104, and such tools may be guided through the tool channel 168 of the catheter body to perform an intraluminal procedure from the distal end of the catheter 104.

The tool may be a medical tool such as an endoscope camera, forceps, a needle, or other biopsy or ablation tools. The tool may be described as an operation tool or working tool. The working tool is inserted or removed through the working tool access port 126. In the embodiments below, at least one embodiment of using a steerable catheter 104 to guide a tool to a target is explained. The tool may include an endoscope camera or an end effector tool, which may be guided through a steerable catheter under the same principles. In a procedure there is usually a planning procedure, a registration procedure, a targeting procedure, and an operation procedure.

The one or more processors, such as, but not limited to, the display controller 100, may generate and output a navigation screen to the one or more displays 101-1, 101-2 based on the 2D/3D model and the position/orientation/navigation/pose/state (or other state) information by executing the software. The navigation screen may indicate a current position/orientation/navigation/pose/state (or other state) of the continuum robot 104 on the 2D/3D model. By using the navigation screen, a user may recognize the current position/orientation/navigation/pose/state (or other state) of the continuum robot 104 in the branching structure. Any feature herein may be used with any navigation/pose/state feature(s) or other feature(s) discussed in International Patent Application No. PCT/US2024/031766, filed May 30, 2024, the disclosure of which is incorporated by reference herein in its entirety.

Any feature of the present disclosure may be used with any navigation/pose/state feature(s) and/or other feature(s) discussed in: International Patent Application No. PCT/US2024/037935, filed Jul. 12, 2024, the disclosure of which is incorporated by reference herein in its entirety; International Patent Application No. PCT/US2024/037924, the disclosure of which is incorporated by reference herein in its entirety; and International Patent Application No. PCT/US2024/037930, the disclosure of which is incorporated by reference herein in its entirety.

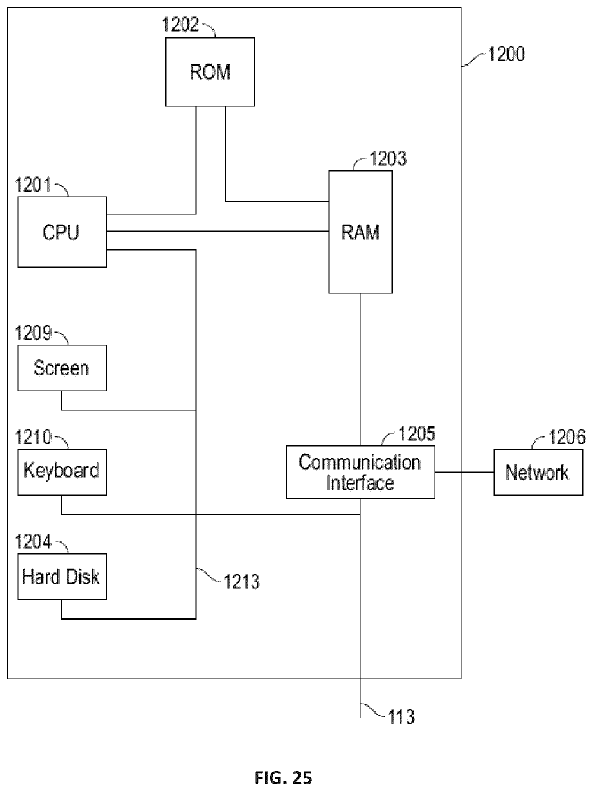

In one or more embodiments, the one or more processors, such as, but not limited to, the display controller 100 and/or the controller 102, may include, as shown in FIG. 5, at least one storage Read Only Memory (ROM) 110, at least one central processing unit (CPU) 120, at least one Random Access Memory (RAM) 130, at least one input and output (I/O) interface 140 and at least one Hard Disc Drive (HDD) 150 (see e.g., also data storage 150 of FIG. 4). A Solid State Drive (SSD) may be used instead of HDD 150 as the data storage 150. In one or more additional embodiments, the one or more processors, and/or the display controller 100 and/or the controller 102, may include structure as shown in any of FIGS. 25-31 as further discussed below.

The ROM 110 and/or HDD 150 operate to store software in one or more embodiments. The RAM 130 may be used as a work memory. The CPU 120 may execute the software program developed in the RAM 130. The I/O 140 operates to input the positional (or other state) information to the display controller 100 (and/or any other processor discussed herein) and to output information for displaying the navigation screen to the one or more displays 101-1, 101-2. In the embodiments below, the navigation screen may be generated by the software program. In one or more other embodiments, the navigation screen may be generated by a firmware.

One or more devices or systems, such as the system 1000, may include a tip position/orientation/navigation/pose/state (or other state) detector 107 that operates to detect a position/orientation/navigation/pose/state (or other state) of the EM tracking sensor 106 and to output the detected positional (and/or other state) information to the controller 100 or 102 (e.g., as shown in FIGS. 1-2), or to any other processor(s) discussed herein.

The controller 102 may operate to receive the positional (or other state) information of the tip of the continuum robot 104 from the camera 106 and/or from the tip position/orientation/navigation/pose/state (or any other state discussed herein) detector 107. In one or more embodiments, the detector 107 may be optional such that the camera 106 may be used alone (e.g., without the tracking sensor 106 and the detector 107). The controller 100 and/or the controller 102 operates to control the actuator 103 in accordance with the manipulation by a user (e.g., manually), and/or automatically (e.g., by a method or methods run by one or more processors using software, by the one or more processors, using automatic manipulation in combination with one or more manual manipulations or adjustments, etc.) via one or more operation/operating portions or operational controllers 105 (e.g., such as, but not limited to a joystick as shown in FIGS. 1-2; see also, diagram of FIG. 4). The one or more displays 101-1, 101-2 and/or operation portion or operational controllers 105 may be used as a user interface 3000 (also referred to as a receiving device) (e.g., as shown diagrammatically in FIG. 4). In an embodiment shown in FIGS. 1-2 or the embodiment shown in FIG. 4, the system(s) 1000 may include, as an operation unit, the display 101-1 (e.g., such as, but not limited to, a large screen user interface with a touch panel, first user interface unit, etc.), the display 101-2 (e.g., such as, but not limited to, a compact user interface with a touch panel, a second user interface unit, etc.) and the operating portion 105 (e.g., such as, but not limited to, a joystick shaped user interface unit having shift lever/button, a third user interface unit, a gamepad, or other input device, etc.).

The controller 100 and/or the controller 102 (and/or any other processor discussed herein) may control the continuum robot 104 based on an algorithm known as follow the leader (FTL) algorithm. The FTL algorithm may be used in addition to the navigation planning and/or autonomous navigation features of the present disclosure. For example, by applying the FTL algorithm, the middle section and the proximal section (following sections) of the continuum robot 104 may move at a first position (or other state) in the same or similar way as the distal section moved at the first position (or other state) or a second position (or state) near the first position (or state) (e.g., during insertion of the continuum robot/catheter 104, by using the navigation planning, autonomous navigation, movement, and/or control feature(s) of the present disclosure, etc.). Similarly, the middle section and the distal section of the continuum robot 104 may move at a first position or state in the same/similar/approximately similar way as the proximal section moved at the first position or state or a second position or state near the first position (e.g., during removal of the continuum robot/catheter 104). Additionally or alternatively, the continuum robot/catheter 104 may be removed by automatically and/or manually moving along the same or similar, or approximately same or similar, path that the continuum robot/catheter 104 used to enter a target (e.g., a body of a patient, an object, a specimen (e.g., tissue), etc.) using the FTL algorithm, including, but not limited to, using FTL with the one or more adjustment, correction, state, and/or smoothing technique(s) discussed herein.

Any of the one or more processors, such as, but not limited to, the controller 102 and the display controller 100, may be configured separately. As aforementioned, the controller 102 may similarly include a CPU 120, a RAM 130, an I/O 140, a ROM 110, and a HDD 150 as shown diagrammatically in FIG. 5. Alternatively, any of the one or more processors, such as, but not limited to, the controller 102 and the display controller 100, may be configured as one device (for example, the structural attributes of the controller 100 and the controller 102 may be combined into one controller or processor, such as, but not limited to, the one or more other processors discussed herein (e.g., computer, console, or processor 1200, 1200′, etc.).

The system 1000 may include a tool channel 126 for a camera, biopsy tools, or other types of medical tools (as shown in FIGS. 1-2). For example, the tool may be a medical tool, such as an endoscope, a forceps, a needle or other biopsy tools, etc. In one or more embodiments, the tool may be described as an operation tool or working tool. The working tool may be inserted or removed through a working tool insertion slot 126 (as shown in FIGS. 1-2). Any of the features of the present disclosure may be used in combination with any of the features, including, but not limited to, the tool insertion slot, as discussed in U.S. Pat. Pub. No. 2024/0112407 A1, published Apr. 4, 2024, the disclosure of which is incorporated by reference herein in its entirety.

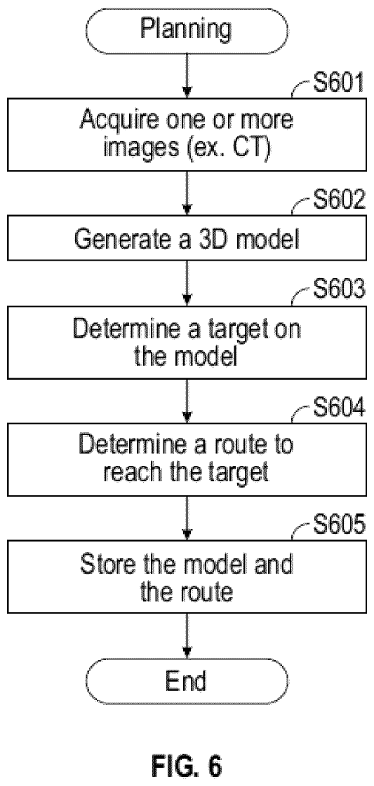

One or more of the features discussed herein may be used for planning procedures, including using one or more models for artificial intelligence applications. As an example of one or more embodiments, FIG. 6 is a flowchart showing steps of at least one planning procedure of an operation of the continuum robot/catheter device 104. One or more of the processors discussed herein may execute the steps shown in FIG. 6, and these steps may be performed by executing a software program read from a storage medium, including, but not limited to, the ROM 110 or HDD 150, by CPU 120 or by any other processor discussed herein. One or more methods of planning using the continuum robot/catheter device 104 may include one or more of the following steps: (i) In step s601, one or more images such, as CT or MRI images, may be acquired; (ii) In step s602, a three dimensional model of a branching structure (for example, an airway model of lungs or a model of an object, specimen or other portion of a body) may be generated based on the acquired one or more images; (iii) In step s603, a target on the branching structure may be determined (e.g., based on a user instruction, based on preset or stored information, etc.); (iv) In step s604, a route of the continuum robot/catheter device 104 to reach the target (e.g., on the branching structure) may be determined (e.g., based on a user instruction, based on preset or stored information, based on a combination of user instruction and stored or preset information, etc.); (v) In step s605, the generated model (e.g., the generated two-dimensional or three-dimensional model) and the decided route on the model may be stored (e.g., in the RAM 130 or HDD or data storage 150, in any other storage medium discussed herein, in any other storage medium known to those skilled in the art, etc.). In this way, a model (e.g., a 2D or 3D model) of a branching structure may be generated, and a target and a route on the model may be determined and stored before the operation of the continuum robot 104 is started.

Embodiments of using a catheter device/continuum robot 104 are explained, such as, but not limited to features for performing navigation planning, autonomous driving/navigation, movement detection, and/or control technique(s).

The system controller 102 (or any other controller, processor, computer, etc. discussed herein) may operate to perform a navigation planning mode and/or an autonomous navigation mode. During the navigation planning mode and/or the autonomous navigation mode, the user does not need to control the bending and translational insertion position of the steerable catheter 104. The navigation planning and/or autonomous navigation mode may include or comprise: (1) a perception step, (2) a planning step, and (3) a control step. In the perception step, the system controller 102 may receive an endoscope view (or imaging data) and may analyze the endoscope view (or imaging data) to find addressable airways from the current position/orientation of the steerable catheter 104. At an end of this analysis, the system controller 102 identifies or perceives these addressable airways as paths in the endoscope view (or imaging data).

The planning step is a step to determine a target path, which is the destination for the steerable catheter 104. While there are a couple of different approaches to select one of the paths as the target path, the present disclosure uniquely includes means to reflect user instructions concurrently for the decision of a target path among the identified or perceived paths. Once the system 1000 determines the target paths while considering concurrent user instructions, the target path is sent to the next step, i.e., the control step.

The control step is a step to control the steerable catheter 104 and the linear translation stage 122 (or any other portion of the robotic platform 108) to navigate the steerable catheter 104 to the target path, pose, state, etc. This step may also be performed as an automatic step. The system controller 102 operates to use information relating to the real time endoscope view (e.g., the view 134), the target path, and an internal design & status information on the robotic catheter system 1000.

Through these three steps, the robotic catheter system 1000 may navigate the steerable catheter 104 autonomously, which achieves reflecting the user's intention efficiently.

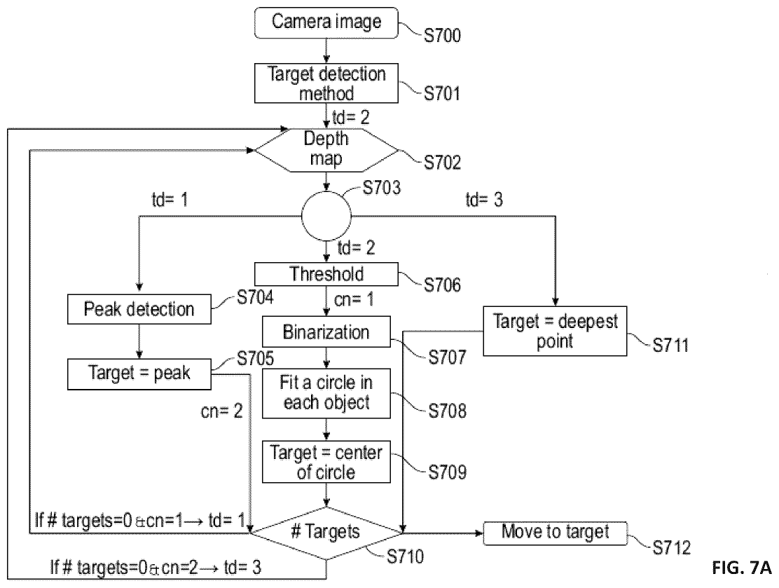

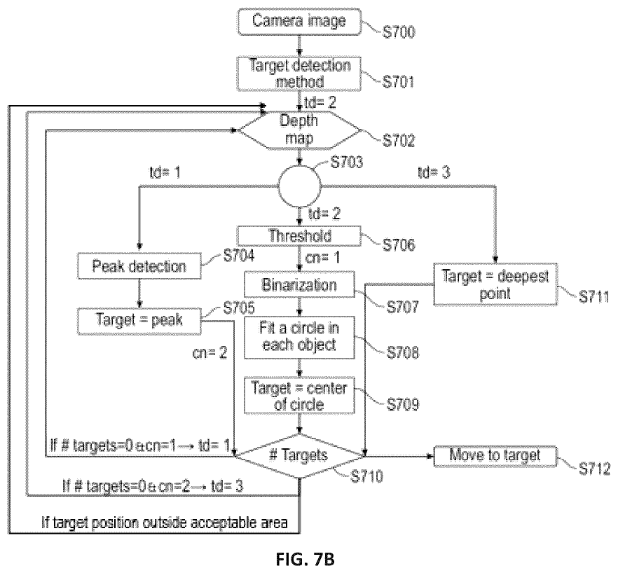

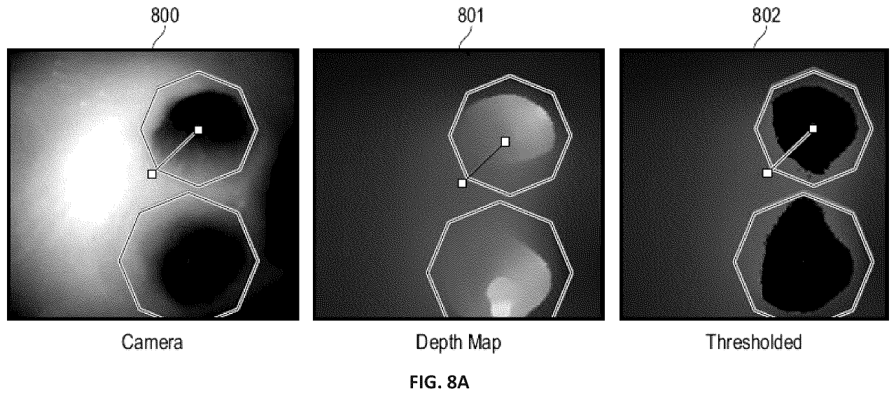

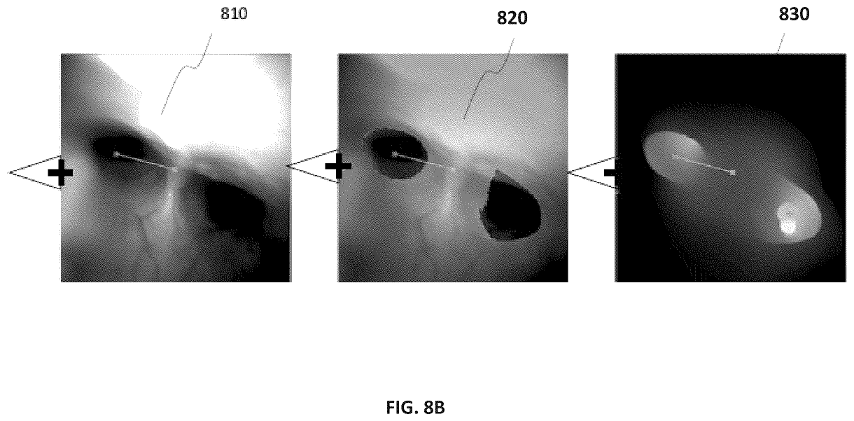

As shown in FIG. 1, the real-time endoscope view 134 may be displayed in a main display 101-1 (as a user input/output device) in the system 1000. The user may see the airways in the real-time endoscope view 134 through the main display 101-1. This real-time endoscope view 134 may also be sent to the system controller 102. In the perception step, the system controller 102 may process the real-time endoscope view 134 and may identify path candidates by using image processing algorithms. Among these path candidates, the system controller 102 may select the paths with the designed computation processes, and then may display the paths with a circle, octagon, or other geometric shape (e.g., one or more set or predetermined geometric shapes or one or more circles, rectangles, squares, ovals, octagons, triangles, any other shape discussed herein or known to those skilled in the art, any closed shape discussed herein or known to those skilled in the art, etc.) with the real-time endoscope view 134 as discussed further below for FIGS. 7A-8B.

In planning step, the system controller 102 may provide a cursor so that the user may indicate the target path by moving the cursor with the joystick 105. When the cursor is disposed or is located within the area of the path, the system controller 102 operates to recognize the path with the cursor as the target path.

In a further embodiment example, the system controller 102 may can pause the motion of the actuator unit 103 and the linear translation stage 122 while the user is moving the cursor so that the user may select the target path with a minimal change of the real-time endoscope view 134 and paths since the system 1000 would not move in such a scenario. Additionally or alternatively, the features of the present disclosure may be performed using artificial intelligence, including the autonomous driving mode. For example, deep learning may be used for performing autonomous driving using deep learning for localization. Any features of the present disclosure may be used with artificial intelligence features discussed in J. Sganga, D. Eng, C. Graetzel, and D. B. Camarillo, “Autonomous Driving in the Lung using Deep Learning for Localization,” July 2019, Accessed: Jun. 28, 2023. [Online]. Available: https://arxiv.org/abs/1907.08136v1, the disclosure of which is incorporated by reference herein in its entirety.

In one or more embodiments, the system controller 102 (or any other controller, processor, computer, etc. discussed herein) may operate to perform a depth map mode. A depth map may be generated or obtained from one or more images (e.g., bronchoscopic images, CT images, images of another imaging modality, etc.). A depth of each image may be identified or evaluated to generate the depth map or maps. The generated depth map or maps may be used to perform navigation planning, autonomous navigation, movement detection, and/or control of a continuum robot, a steerable catheter, an imaging device or system, etc. as discussed herein. In one or more embodiments, thresholding may be applied to the generated depth map or maps, or to the depth map mode, to evaluate accuracy for navigation purposes. For example, while not limited to only this type of a threshold, a threshold may be set for an acceptable distance between the ground truth (and/or a target camera location, a predetermined camera location, an actual camera location, etc.) and an estimated camera location for a catheter or continuum robot (e.g., the catheter or continuum robot 104). By way of a further example, the threshold may defined such that the distance between the ground truth (and/or a target camera location, a predetermined camera location, an actual camera location, etc.) and an estimated camera location is equal to or less than, or less than, a set or predetermined distance of one or more of the following: 5 mm, 10 mm, about 5 mm, about 10 mm, any other distance set by a user of the device (depending on a particular application). In one or more embodiments, the predetermined distance may be less than 5 mm or less than about 5 mm. Any other type of thresholding may be applied to the depth mapping to improve and/or confirm the accuracy of the depth map(s).

Additionally or alternatively, thresholding may be applied to segment the one or more images to help identify or find one or more objects and to ultimately help define one or more targets used for the navigation planning, autonomous navigation, movement detection, and/or control features of the present disclosure. For example, a depth map or maps may be created or generated using one or more images (e.g., CT images, bronchoscopic images, images of another imaging modality, vessel images, etc.), and then, by applying a threshold to the depth map, the objects in the one or more images may be segmented (e.g., a lung may be segmented, one or more airways may be segmented, etc.). In one or more embodiments, the segmented portions of the one or more images (e.g., the one or more segmented airways, the segmented portions of a lung, etc.) may define one or more navigation targets for a next automatic robotic movement, navigation, and/or control. Examples of segmented airways are discussed further below with respect to FIGS. 8A-8B. In one or more embodiments, one or more of the automated methods that may be used to apply thresholding may include one or more of the following: a watershed method (such as, but not limited to, watershed method(s) discussed in L. J. Belaid and W. Mourou, “IMAGE SEGMENTATION: A WATERSHED TRANSFORMATION ALGORITHM,” 2011, vol. 28, no. 2, p. 10, 2011, doi: 10.5566/ias.v28. p 93-102, which is incorporated by reference herein in its entirety), a k-means method (such as, but not limited to, k-means method(s) discussed in T. Kanungo, D. M. Mount, N. S. Netanyahu, C. D. Piatko, R. Silverman, and A. Y. Wu, “An efficient k-means clustering algorithm: Analysis and implementation,” IEEE Trans Pattern Anal Mach Intell, vol. 24, no. 7, pp. 881-892, 2002, doi: Doi 10.no9/Tpami.2002.1017616, which is incorporated by reference herein in its entirety), an automatic threshold method (such as, but not limited to, automatic threshold method(s) discussed in N. Otsu, “Threshold Selection Method from Gray-Level Histograms,” IEEE Trans SystMan Cybern, vol. 9, no. 1, pp. 62-66, 1979, which is incorporated by reference herein in its entirety) using a sharp slope method (such as, but not limited to, sharp slope method(s) discussed in U.S. Pat. Pub. No. 2023/0115191 A1, published on Apr. 13, 2023, which is incorporated by reference herein in its entirety) and/or any combination of the subject methods. In one or more embodiments, peak detection may include any of the techniques discussed herein, including, but not limited to, the techniques discussed in at least “8 Peak detection,” Data Handling in Science and Technology, vol. 21, no. C, pp. 183-190, January 1998, doi: 10.1016/S0922-3487(98)80027-0, which is incorporated by reference herein in its entirety.

The depth map(s) may be obtained, and/or the quality of the obtained depth map(s) may be evaluated, using artificial intelligence structure, such as, but not limited, convolutional neural networks, generative adversarial networks (GANs), neural networks, any other AI structure or feature(s) discussed herein, any other AI network structure(s) known to those skilled in the art, etc. For example, a generator of a generative adversarial network may operate to generate an image(s) that is/are so similar to ground truth image(s) that a discriminator of the generative adversarial network is not able to distinguish between the generated image(s) and the ground truth image(s). The generative adversarial network may include one or more generators and one or more discriminators. Each generator of the generative adversarial network may operate to estimate depth of each image (e.g., a CT image, a bronchoscopic image, etc.), and each discriminator of the generative adversarial network may operate to determine whether the estimated depth of each image (e.g., a CT image, a bronchoscopic image, etc.) is estimated (or fake) or ground truth (or real). In one or more embodiments, an AI network, such as, but not limited to, a GAN or a consistent GAN (cGAN), may receive an image or images as an input and may obtain or create a depth map for each image or images. In one or more embodiments, an AI network may evaluate obtained one or more images (e.g., a CT image, a bronchoscopic image, etc.), one or more virtual images, and one or more ground truth depth maps to generate depth map(s) for the one or more images and/or evaluate the generated depth map(s). A Three Cycle-Consistent Generative Adversarial Network (3cGAN) may be used to obtain the depth map(s) and/or evaluate the quality of the depth map(s), and an unsupervised learning method (designed and trained in an unsupervised procedure) may be employed on the depth map(s) and the one or more images (e.g., a CT image or images, a bronchoscopic image or images, any other obtained image or images, etc.). Any feature or features of obtaining a depth map or performing a depth map mode of the present disclosure may be used with any of the depth map or depth estimation features as discussed in A. Banach, F. King, F. Masaki, H. Tsukada, and N. Hata, “Visually Navigated Bronchoscopy using three cycle-Consistent generative adversarial network for depth estimation,” Med Image Anal, vol. 73, p. 102164, October 2021, doi: 10.1016/J.MEDIA.2021.102164 (Banach 2021) or the pose estimates or tracking as described in U.S. Prov. Patent Application No. 63/714,676 entitled” Self-Supervised Direct Pose Estimation for Vision-Based Tracking in Navigated Bronchoscopy” filed Oct. 31, 2024 (Kalia 2024), the disclosures of which are incorporated by reference herein in their entireties.

In one or more embodiments, the system controller 102 (or any other controller, processor, computer, etc. discussed herein) may operate to perform a computation of one or more lumen (e.g., a lumen computation mode) and/or one or more of the following: a one or more set or predetermined geometric shapes or one or more circles, rectangles, squares, ovals, octagons, and/or triangles fit/blob process, a peak detection, and/or a deepest point analysis. In one or more embodiments, the computation of one or more lumen may include a one or more set or predetermined geometric shapes or one or more circles, rectangles, squares, ovals, octagons, and/or triangles fit/blob process, a peak detection, and/or a deepest point analysis.

For a one or more set or predetermined geometric shapes or one or more circles, rectangles, squares, ovals, octagons, and/or triangles/blob fit technique(s) of the present disclosure, fitting one or more set or predetermined geometric shapes or one or more circles, rectangles, squares, ovals, octagons, and/or triangles or a blob to a binary object may be equivalent to fitting one or more set or predetermined geometric shapes or one or more circles, rectangles, squares, ovals, octagons, and/or triangles or a blob to a set of points. In one or more embodiments, the set of points may be the boundary points of the binary object. Given a set of points (x1,y1),(x2,y2),(x3,y3), . . . , (xn,yn) a circle (x−a)²+(y−b)²=c² may be fit to the points by summing the squares of the distances from the points to the circle:

$$SS ( a , b , c ) = {\sum }_{k = 1}^{n} {( c - \sqrt{{( xi - a )}^{2} + {( yi - b )}^{2}} )}^{2} .$$

However, a circle/blob fit is not limited thereto (as discussed herein, any one or more set or predetermined geometric shapes or one or more circles, rectangles, squares, ovals, octagons, and/or triangles (or other shape(s)) may be used). Indeed, there are several other variations that may be applied as described in D. Umbach and K. N. Jones, “A few methods for fitting circles to data,” in IEEE Transactions on Instrumentation and Measurement, vol. 52, no. 6, pp. 1881-1885, December 2003, doi: 10.1109/TIM.2003.820472, the disclosure of which is incorporated by reference herein in its entirety. For example, circle/Blob fitting may be achieved on the binary objects by calculating their circularity/blob shape as 4πArea/(perimeter)² and then defining the circle/blob radius.

For peak detection and/or deepest point technique(s), one or more embodiments may include same in various ways. For example, peak detection may be performed in a 1-D signal and may be defined as the extreme value of the signal. Similarly, 2-D image peak detection may be defined as the highest value of the 2-D matrix. Herein, a depth map or maps is/are the 2-D matrix in one or more embodiments, and a peak is the highest value of the depth math or maps which may correspond to the deepest point. However, since there might be more than one airway or airways which are represented by different depth value concentrations along the depth map(s) image or images, more than one peak may exist. The depth map or maps produce an image which predicts the depth of the airways; therefore, for each airway, there may be a concentration of non-zero pixels around a deepest point that the neural network, residual network, GANs, or any other AI structure/network discussed herein or known to those skilled in the art predicted. By applying peak detection to all the non-zero concentrations of the 2-D depth map or maps, the peak of each concentration is detected; each peak corresponds to an airway. In one or more embodiments (including the study discussed herein), a GANs (or another AI structure/network) may be used (or was used) to predict the concentration of non-zero pixels.

One or more features discussed herein may be used for performing navigation planning, autonomous navigation, movement detection, and/or control technique(s) for a steerable catheter, continuum robot, imaging device or system, etc. as discussed herein. FIG. 7A is a flowchart showing steps of at least one procedure for performing navigation planning, autonomous navigation, movement detection, and/or control technique(s) for a continuum robot/catheter device (e.g., such as continuum robot/catheter device 104). One or more of the processors discussed herein, one or more AI networks discussed herein, and/or a combination thereof may execute the steps shown in FIG. 7A, and these steps may be performed by executing a software program read from a storage medium, including, but not limited to, the ROM 110 or HDD 150, by CPU 120 or by any other processor discussed herein. While not limited thereto, one or more methods of performing navigation planning, autonomous navigation, movement detection, and/or control technique(s) for a catheter or probe of a continuum robot device or system may include one or more of the following steps: (i) in step S700, one or more images (e.g., one or more camera images, one or more CT images (or images of another imaging modality), one or more bronchoscopic images, etc.) are obtained; (ii) in step S701, a target detection method is selected (automatically or manually) (e.g., target detection (td)=1 for the peak detection method or mode, td=2 for the thresholding method or mode, td=3 for the deepest point method or mode, etc.—the target detection methods shown in FIG. 7A are illustrative, are not limited thereto, and may be exchanged or substitute or used along with any combination of detection methods discussed in the present disclosure or known to those skilled in the art); (iii) in step S703, based on the td value, the method continues to perform the selected target detection method and proceeds to step S704 for the peak detection method or mode, to step S706 for the thresholding method or mode, or to step S711 for the deepest point method or mode; (iv) in a case where td=1, the peak detection method or mode is performed in step S704, a target or targets are set to be the detected peak or peaks in step S705 and a counter (en) is set to 2 (cn=2), and a number of targets is evaluated in step S710 such that, in a case where no targets are found (# targets=0) and the counter=2, then td is set to a value of 3 and the process returns to the depth map step S702 and proceeds to step S711 for the deepest point method or mode; (v) in a case where td=2, the thresholding method or mode is performed in step S706 to identify one or more objects, the counter is set to be equal to 1(cn=1), binarization is performed in step S707 to process the image data (e.g., the image data may be converted from color to black and white images, the image data may be split into data sets, etc.), fitting one or more set or predetermined geometric shapes or one or more circles, rectangles, squares, ovals, octagons, and/or triangles in or on each object is performed in step S708 (also referred to as a blob fit or blob detection method), a target or targets is/are set to be at a predetermined or set location (e.g., a center) of the one or more set or predetermined geometric shapes or the one or more circles, rectangles, squares, ovals, octagons, and/or triangles for each object of the one or more objects in step S709, a number of targets is evaluated in step S710 such that, in a case where no targets are found (# targets=0) and the counter=1, then td is set to a value of 1 and the process returns to the depth map step S702 and proceeds to step S704 for the peak detection method or mode in step S704, a target or targets is/are identified as the detected peak or peaks in step S705, and a number of targets is evaluated in step S710; and (vi) in a case where the number of targets evaluated in step S710 is 1 or more, then the process proceeds to step S712 where the continuum robot or steerable catheter (or other imaging device or system) (e.g., the continuum robot or steerable catheter 104) is moved to the target or targets. In one or more embodiments (see e.g., FIG. 7B), the method may further include (after step (vi)) the following: (vii) in a case where a target position is disposed or located outside of an acceptable or predetermined area, then the process returns to the depth map step S702. In one or more embodiments, the steps S701 through S712 of FIGS. 7A and/or 7B may be performed again for an obtained or received next image or images to evaluate the next movement, pose, position, orientation, or state for the navigation planning, autonomous navigation, movement detection, and/or control of the continuum robot or steerable catheter (or imaging device or system) 104. In step S702, the method may estimate (automatically or manually) the depth map or maps (e.g., a 2D or 3D depth map or maps) of one or more images. The one or more depth maps may be estimated or determined using any technique discussed herein, including, but not limited to, artificial intelligence. For example, any AI network, including, but not limited to a neural network, a convolutional neural network, a generative adversarial network, any other AI network or structure discussed herein or known to those skilled in the art, etc., may be used to estimate or determine the depth map or maps (e.g., automatically). The use of a target detection method value (e.g., td=1, td=2, td=3) and/or a counter (en=1 or cn=2) is illustrative, and the navigation planning, autonomous navigation, movement detection, and/or control technique(s) of the present disclosure are not limited thereto. For example, in one or more embodiments, a counter may not be used and/or a target detection method value may not be used such that at least one embodiment may iteratively perform a target detection method of a plurality of target detection methods and move on and use the next target detection method of the plurality of the target detection methods until a target or targets is/are found. Alternatively or additionally, even in a case where a target or targets has/have been found already using a particular target detection method, one or more embodiments may continue to use one or more of the other target detection methods (or any combination of the plurality of target detection methods or modes) to confirm and/or evaluate the accuracy and/or results of the target detection method or mode used to find the already-identified one or more targets. In other words, the identified one or more targets may be double checked, triple checked, etc. In one or more embodiments, the deepest point method or mode of step S711 may be used as a backup to identify a target or targets in a case where other target detection methods do not find any targets (# targets=0). Additionally or alternatively, one or more steps of FIGS. 7A and/or 7B, such as, but not limited to step S707 for binarization, may be omitted in one or more embodiments.

In one or more embodiments, a non-transitory computer-readable storage medium may store at least one program for causing a computer to execute a method for performing navigation planning, autonomous navigation, movement detection, and/or control of a continuum robot or catheter, the method comprising one or more of the following steps: (i) in step S700, one or more images (e.g., one or more camera images, one or more CT images (or images of another imaging modality), one or more bronchoscopic images, etc.) are obtained; (ii) in step S701, a target detection method is selected (automatically or manually) (e.g., target detection (td)=1 for the peak detection method or mode, td=2 for the thresholding method or mode, td=3 for the deepest point method or mode, etc.—the target detection methods shown in FIGS. 7A-7B are illustrative, are not limited thereto, and may be exchanged or substitute or used along with any combination of detection methods discussed in the present disclosure or known to those skilled in the art); (iii) in step S703, based on the td value, the method continues to perform the selected target detection method and proceeds to step S704 for the peak detection method or mode, to step S706 for the thresholding method or mode, or to step S711 for the deepest point method or mode; (iv) in a case where td=1, the peak detection method or mode is performed in step S704, a target or targets are set to be the detected peak or peaks in step S705 and a counter (en) is set to 2 (cn=2), and a number of targets is evaluated in step S710 such that, in a case where no targets are found (# targets=0) and the counter=2, then td is set to a value of 3 and the process returns to the depth map step S702 and proceeds to step S711 for the deepest point method or mode; (v) in a case where td=2, the thresholding method or mode is performed in step S706 to identify one or more objects, the counter is set to be equal to 1 (cn=1), binarization is performed in step S707 to process the image data (e.g., the image data may be converted from color to black and white images, the image data may be split into data sets, etc.), fitting one or more set or predetermined geometric shapes or one or more circles, rectangles, squares, ovals, octagons, and/or triangles in or on each object is performed in step S708 (also referred to as a blob fit or blob detection method), a target or targets is/are set to be at a predetermined or set location (e.g., a center) of the one or more set or predetermined geometric shapes or the one or more circles, rectangles, squares, ovals, octagons, and/or triangles for each object of the one or more objects in step S709, a number of targets is evaluated in step S710 such that, in a case where no targets are found (# targets=0) and the counter=1, then td is set to a value of 1 and the process returns to the depth map step S702 and proceeds to step S704 for the peak detection method or mode in step S704, a target or targets is/are identified as the detected peak or peaks in step S705, and a number of targets is evaluated in step S710; and (vi) in a case where the number of targets evaluated in step S710 is 1 or more, then the process proceeds to step S712 where the continuum robot or steerable catheter (or other imaging device or system) (e.g., the continuum robot or steerable catheter 104) is moved to the target or targets. In one or more embodiments, the steps S701 through S712 of FIGS. 7A and/or 7B may be performed again for an obtained or received next image or images to evaluate the next movement, pose, position, orientation, or state for the navigation planning, autonomous navigation, movement detection, and/or control of the continuum robot or steerable catheter (or imaging device or system) 104. In step S702, the method may estimate (automatically or manually) the depth map or maps (e.g., a 2D or 3D depth map or maps) of one or more images. The one or more depth maps may be estimated or determined using any technique discussed herein, including, but not limited to, artificial intelligence. For example, any AI network, including, but not limited to a neural network, a convolutional neural network, a generative adversarial network, any other AI network or structure discussed herein or known to those skilled in the art, etc., may be used to estimate or determine the depth map or maps (e.g., automatically). The use of a target detection method value (e.g., td=1, td=2, td=3) and/or a counter (en=1 or cn=2) is illustrative, and the navigation planning, autonomous navigation, movement detection, and/or control technique(s) of the present disclosure are not limited thereto. For example, in one or more embodiments, a counter may not be used and/or a target detection method value may not be used such that at least one embodiment may iteratively perform a target detection method of a plurality of target detection methods and move on and use the next target detection method of the plurality of the target detection methods until a target or targets is/are found. Alternatively or additionally, even in a case where a target or targets has/have been found already using a particular target detection method, one or more embodiments may continue to use one or more of the other target detection methods (or any combination of the plurality of target detection methods or modes) to confirm and/or evaluate the accuracy and/or results of the target detection method or mode used to find the already-identified one or more targets. In other words, the identified one or more targets may be double checked, triple checked, etc. In one or more embodiments, the deepest point method or mode of step S711 may be used as a backup to identify a target or targets in a case where other target detection methods do not find any targets (# targets=0). Additionally or alternatively, one or more steps of FIGS. 7A and/or 7B, such as, but not limited to step S707 for binarization, may be omitted in one or more embodiments. In one or more embodiments where segmentation may be used using three categories (e.g., airways, background, and edges of the image(s), for example), then an image may have three colors. As such, binarization may be useful to convert the image to black and white image data to perform processing on same as discussed herein. Such processing may be useful to improve processing speed and accuracy.

FIG. 8A shows images of at least one embodiment of an application example of navigation planning, autonomous navigation, and/or control technique(s) and movement detection for a camera view 800 (left), a depth map 801 (center), and a thresholded image 802 (right) in accordance with one or more aspects of the present disclosure. A depth map may be created using the bronchoscopic images and then, by applying a threshold to the depth map, the airways may be segmented. The segmented airways shown in thresholded image 802 may define the navigation targets (shown in the octagons of image 802) of the next automatic robotic movement. In one or more embodiments, the continuum robot or steerable catheter 104 may follow the target(s) (which a user may change by dragging and dropping the target(s) (e.g., a user may drag and drop an identifier for the target, the user may drag and drop a cross or an x element representing the location for the target, etc.) in one or more embodiments), and the continuum robot or steerable catheter 104 may move forward and rotate on its own while targeting a predetermined location (e.g., a center) of the target(s) of the airway. In one or more embodiments, the depth map (see e.g., in image 801) may be processed with any combination of blob/one or more set or predetermined geometric shapes or one or more circles, rectangles, squares, ovals, octagons, and/or triangles fit, peak detection, and/or deepest point methods or modes to detect the airways that are segmented. As aforementioned, the detected airways may define the navigation targets of the next automatic robotic movement. In a case where a cross or identifier is used for the target(s), the continuum robot or steerable catheter 104 may move in a direction of the airway with its center closer to the cross or identifier. The continuum robot or steerable catheter 104 may move forward and may rotate in an autonomous fashion targeting the center of the airway (or any other designated or set point or area of the airway) in one or more embodiments. A circle fit algorithm is discussed herein for one or more embodiments. The circle shape provides an advantage in that it has a low computational burden, and the lumen within a lung may be substantially circular. However, as discussed herein, other geometric shapes may be used or preferred in a number of embodiments. For example, as may be seen in the camera view 800 in FIG. 8A, the lumen are more oval than circular, so an oval geometric shape may be used or preferred. The apparatuses, systems, methods, and/or other features of the present disclosure may be optimized to other geometries as well, depending on the particular application(s) embodied or desired. For example, one or more airways may be deformed due to one or more reasons or conditions (e.g., environmental changes, patient diagnosis, structural specifics for one or more lungs or other objects or targets, etc.). In addition, while the circle fit may be used for the planning shown in FIG. 8A, this figure shows an octagon defining the fitting of the lumen in the images. Such a difference may help with clarifying the different information being provided in the display. In a case where an indicator of the geometric fit (e.g., a circle fit) may be shown in a display, it may have the same geometry as used in the fitting algorithm, or it may have a different geometry, such as the octagon shown in FIG. 8A.

Additionally, a study was conducted to introduce and evaluate new and non-obvious techniques for achieving autonomous advancement of a multi-section continuum robot within lung airways, driven by depth map perception. By harnessing depth maps as a fundamental perception modality, one or more embodiments of the studied system aims to enhance the robot's ability to navigate and manipulate within the intricate and complex anatomical structure of the lungs (or any other targeted anatomy, object, or sample). The utilization of depth maps enables the robot to accurately perceive its environment, facilitating precise localization, mapping, and obstacle avoidance. This, in turn, helps safer and more effective robot-assisted interventions in pulmonary procedures. Experimental results highlight the feasibility and potential of the depth map-driven approach, showcasing its ability to advance the field of minimally invasive lung surgeries (or other minimally invasive surgical procedures, imaging procedures, etc.).

As aforementioned, continuum robots are flexible systems used in transbronchial biopsy, offering enhanced precision and dexterity. Training these robots is challenging due to their nonlinear behavior, necessitating advanced control algorithms and extensive data collection. Autonomous advancements are crucial for improving their maneuverability.

Sganga, et al. introduced deep learning approaches for localizing a bronchoscope using real-time bronchoscopic video as discussed in J. Sganga, D. Eng, C. Graetzel, and D. Camarillo, “Offsetnet: Deep learning for localization in the lung using rendered images,” in 2019 International Conference on Robotics and Automation (ICRA), 2019, pp. 5046-5052, the disclosure of which is incorporated by reference herein in its entirety. Zou, et al. proposed a method for accurately detecting the lumen center in bronchoscopy images as discussed in Y. Zou, B. Guan, J. Zhao, S. Wang, X. Sun, and J. Li, “Robotic-assisted automatic orientation and insertion for bronchoscopy based on image guidance,” IEEE Transactions on Medical Robotics and Bionics, vol. 4, no. 3, pp. 588-598, 2022, the disclosure of which is incorporated by reference herein in its entirety. However, there are drawbacks to the techniques discussed in the Sganga, et al. and Zou, et al. publications.

This study of the present disclosure aimed to develop and validate the autonomous advancement of a robotic bronchoscope using depth map perception. The approach involves generating depth maps and employing automated lumen detection to enhance the robot's accuracy and efficiency. Additionally, an early feasibility study evaluated the performance of autonomous advancement in lung phantoms derived from CT scans of lung cancer subjects.

Bronchoscopic operations were conducted using a snake robot developed in the researchers' lab (some of the features of which are discussed in F. Masaki, F. King, T. Kato, H. Tsukada, Y. Colson, and N. Hata, “Technical validation of multi-section robotic bronchoscope with first person view control for transbronchial biopsies of peripheral lung,” IEEE Transactions on Biomedical Engineering, vol. 68, no. 12, pp. 3534-3542, 2021, which is incorporated by reference herein in its entirety), equipped with a bronchoscopic camera (OVM6946 OmniVision, CA). The captured bronchoscopic images were transmitted to a control workstation, where depth maps were created using a method involving a Three Cycle-Consistent Generative Adversarial Network (3cGAN) (see e.g., a 3cGAN as discussed in A. Banach, F. King, F. Masaki, H. Tsukada, and N. Hata, “Visually navigated bronchoscopy using three cycle-consistent generative adversarial network for depth estimation,” Medical Image Analysis, vol. 73, p. 102164, 2021. [Online]. Available: https://www.sciencedirect.com/science/article/pii/S1361841521002103, the disclosure of which is incorporated by reference herein in its entirety). A combination of thresholding and blob detection algorithms, methods, or modes was used to detect the airway path, along with peak detection for missed airways.

A control vector was computed from the chosen point of advancement (identified centroid or deepest point) to the center of the depth map image. This control vector represents the direction of movement on the 2D plane of original RGB and depth map images. A software-emulated joystick/gamepad was used in place of the physical interface to control the snake robot (also referred to herein as a continuum robot, steerable catheter, imaging device or system, etc.). The magnitude of the control vector was calculated, and if magnitude fell below a threshold, the robot advanced. If the magnitude exceeded the threshold, the joystick was tilted to initiate bending. This process was repeated using a new image from the Snake Robot interface.

During each trial of the autonomous robotic advancement, the robotic bronchoscope was initially positioned in front of the carina within the trachea. The program was initiated by the operator or user, who possessed familiarity with the software, initiating the robot's movement. The operator's or user's sole task was to drag and drop a green cross within the bronchoscopic image to indicate the desired direction. Visual assessment was used to determine whether autonomous advancement to the intended airway was successfully achieved at each branching point. Summary statistics were generated to evaluate the success rate based on the order of branching generations and lobe segments.

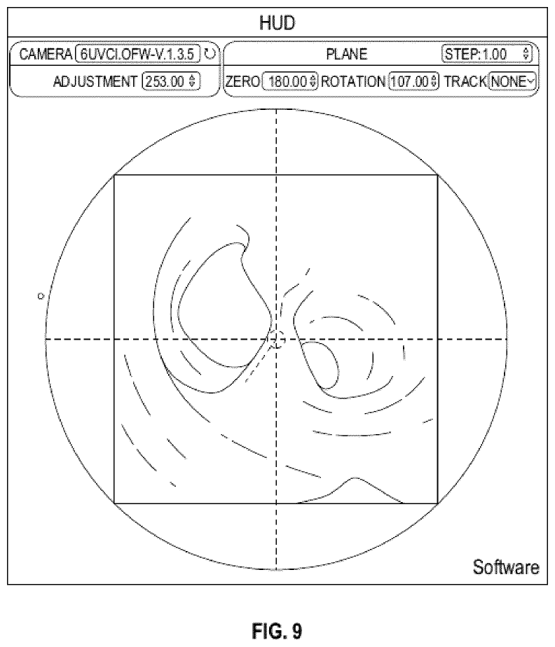

In one or more embodiments of the present disclosure, a device, apparatus, or system may be a continuum robot or a robotic bronchoscope, and one or more embodiments of the present disclosure may employ depth map-driven autonomous advancement of a multi-section continuum robot or robotic bronchoscope in one or more lung airways. Additional non-limiting, non-exhaustive embodiment details for one or more bronchoscope, robotic bronchoscope, apparatus, system, method, storage medium, etc. details, and one or more details for the performed study/studies, are shown in one or more figures, see e.g., at least FIGS. 9-24B.

One or more methods of the present disclosure were validated on one clinically derived phantom and two ex-vivo pig lung specimens with and without simulated breathing motion, resulting in 261 advancement paths in total, and an in vivo animal. The achieved target reachability in phantoms was 73.3%, in ex-vivo specimens without breathing motion was 77% and 78%, and in ex-vivo specimens with breathing motion was 69% and 76%. The in vivo comparison study discussed below showed that the autonomous driving took less time for bending than human operators (the median time at each bifurcation=2.5 and 1.3 [s] for a human operator and autonomous driving, respectively). With the presented methodology(ies) and performance(s), the proposed supervised-autonomous navigation/driving/planning approach(es) in the lung is/are proven to be clinically feasible. By potentially enhancing precision and consistency in tissue sampling, this system or systems have the potential to redefine the standard of care for lung cancer patients, leading to more accurate diagnoses and streamlined healthcare workflows.

### Results

### Robotic Bronchoscope Features for One or More Embodiments and for Performed Study

Bronchoscopic operations were performed using a snake robot developed in (23, 24) with the OVM6946 bronchoscopic camera (OmniVision, CA, USA). The snake robot is a robotic bronchoscope composed of, or including at least, the following parts in one or more embodiments: i) the robotic catheter, ii) the actuator unit, iii) the robotic arm, and iv) the software (see e.g., FIG. 9). The robotic catheter is developed to mimic, and improve upon and outperform, a manual catheter, and, in one or more embodiments, the robotic catheter includes nine drive wires which travel through the steerable catheter, housed within an outer skin made of polyether block amide (PEBA) of 0.13 mm thickness. The catheter includes a central channel which allows for inserting the bronchoscopic camera. The outer and inner diameters (OD, ID) of the catheter are 3 and 1.8 mm, respectively (see e.g., J. Zhang, et al., Nature Communications, vol. 15, no. 1, p. 241 (January 2024), which is incorporated by reference herein in its entirety). The steering structure of the catheter includes two distal bending sections: the tip and middle sections, and one proximal bending section without an intermediate passive section. Each of the sections has its own degree of freedom (DOF) (see e.g., U.S. Pat. Nos. 11,504,501 and 11,559,190 and U.S. Pat. Pub. No. 2021/0369085, the disclosures of which patents and publication(s) are incorporated by reference herein in their entireties). The catheter is actuated through the actuator unit attached to the robotic arm and includes nine motors that control the nine catheter wires. Each motor operates to bend one wire of the catheter by applying pushing or pulling force to the drive wire. Both the robotic catheter and actuator are attached to a robotic arm, including a rail that allows for a linear translation of the catheter. The movement of the catheter over the rail is achieved through a linear stage actuator, which pushes or pulls the actuator and the attached catheter. The catheter, actuator unit, and robotic arm are coupled into a system controller, which allows their communication with the software. While not limited thereto, the robot's movement may be achieved using a handheld controller (gamepad) or, like in this study, through autonomous driving software. The validation design of the robotic bronchoscope was performed by replicating real surgical scenarios, where the bronchoscope entered the trachea and navigated in the airways toward a predefined target (see e.g., L. Dupourqué, et al., International journal of computer assisted radiology and surgery, vo. 14, no. 11, pp. 2021-2029 (2019), which is incorporated by reference herein in its entirety).

### Autonomous Driving

### Perception

The autonomous driving method feature(s) of the present disclosure relies/rely on the 2D image from the monocular bronchoscopic camera without tracking hardware or prior CT segmentation in one or more embodiments. A 200×200 pixel grayscale bronchoscopic image serves as input for a deep learning model (3cGAN (see e.g., Banach 2021, or Kalia 2024, the disclosures of which are incorporated by reference herein in their entireties)) that generates a bronchoscopic depth map and/or pose estimate.

Specifically, 3cGAN's adversarial loss accumulates losses across six levels:

$${L}_{{gan}_{6}} = {L}_{{gan}_{lev}} + {L}_{{gan}_{lev}} + … + {L}_{{gan}_{lev}} \qquad ( 1 )$$

where the adversarial loss of level i is referred to as L_{gan}_{ₗₑᵥ}.

The cycle consistency loss combines the cycle consistency losses from all three level pairs:

$${L}_{{cyc}_{6}} = {‖ {A}_{1} - {G}_{{B}_{2} {A}_{2}^{\hat{}}} ( \hat{{B}_{1}} ) ‖}_{L 2} + {‖ {B}_{2} - {G}_{{A}_{1} {B}_{1}^{\hat{}}} ( \hat{{A}_{2}} ) ‖}_{L 2} + {‖ {C}_{3} - {G}_{{B}_{4} {C}_{4}^{\hat{}}} ( \hat{{B}_{3}} ) ‖}_{L 2} + {‖ {B}_{4} - {G}_{{C}_{3} {B}_{3}^{\hat{}}} ( \hat{{C}_{4}} ) ‖}_{L 2} + {‖ {A}_{5} - {G}_{{C}_{6} {A}_{6}^{\hat{}}} ( \hat{{C}_{5}} ) ‖}_{L 2} + {‖ {C}_{6} - {G}_{{A}_{5} {C}_{5}^{\hat{}}} ( \hat{{A}_{6}} ) ‖}_{L 2} \qquad ( 2 )$$

$${L}_{{cyc}_{6}} = {‖ {A}_{1} - {G}_{{B}_{2} {A}_{2}^{\hat{}}} ( \hat{{B}_{1}} ) ‖}_{L 2} + {‖ {B}_{2} - {G}_{{A}_{1} {B}_{1}^{\hat{}}} ( \hat{{A}_{2}} ) ‖}_{L 2} + {‖ {C}_{3} - {G}_{{B}_{4} {C}_{4}^{\hat{}}} ( \hat{{B}_{3}} ) ‖}_{L 2} + {‖ {B}_{4} - {G}_{{C}_{3} {B}_{3}^{\hat{}}} ( \hat{{C}_{4}} ) ‖}_{L 2} ++ {‖ {A}_{5} - {G}_{{C}_{6} {A}_{6}^{\hat{}}} ( \hat{{C}_{5}} ) ‖}_{L 2} + {‖ {C}_{6} - {G}_{{A}_{5} {C}_{5}^{\hat{}}} ( \hat{{A}_{6}} ) ‖}_{L 2} \qquad ( 2 )$$

where A stands for the bronchoscopic image, B stands for the depth map, C stands for virtual bronchoscopic image, \hat{X} represents estimation of X, and the lower index i stands for the networks level.

The merging loss of the 3cGAN combines all the networks levels:

$${L}_{{m}_{6}} = {‖ {B}_{2} - {G}_{{A}_{1} {B}_{1}^{\hat{}}} ( {G}_{{C}_{6} {A}_{6}^{\hat{}}} ( {G}_{{B}_{4} {C}_{4}^{\hat{}}} ( {G}_{{C}_{3} {B}_{3}^{\hat{}}} ( {G}_{{A}_{5} {C}_{5}^{\hat{}}} ( {G}_{{B}_{2} {A}_{2}^{\hat{}}} ( {B}_{2} ) ) ) ) ‖}_{L 2} \qquad ( 3 )$$

$${L}_{{m}_{6}} = {‖ {B}_{2} - {G}_{{A}_{1} {B}_{1}^{\hat{}}} ( {G}_{{C}_{6} {A}_{6}^{\hat{}}} ( {G}_{{B}_{4} {C}_{4}^{\hat{}}} ( {G}_{{C}_{3} {B}_{3}^{\hat{}}} ( {G}_{{A}_{5} {C}_{5}^{\hat{}}} ( {G}_{{B}_{2} {A}_{2}^{\hat{}}} ( {B}_{2} ) ) ) ) ‖}_{L 2} \qquad ( 3 )$$

The total loss function of the 3cGAN is:

$${L}_{6} = {L}_{{gan}_{6}} + {L}_{{cyc}_{6}} + {L}_{{m}_{6}} \qquad ( 4 )$$

The 3cGAN model underwent unsupervised training using bronchoscopic images from phantoms derived from segmented airways. Bronchoscopic operations to acquire the training data were performed using a Scope 4 bronchoscope (Ambu Inc, Columbia, MD), while virtual bronchoscopic images and ground truth depth maps were generated in Unity (Unity Technologies, San Francisco, CA). The training ex-vivo dataset contained 2458 images. The network was trained in PyTorch using an Adam optimizer on 50 epochs with a learning rate of 2-10-4 and a batch size of one. Training time was approximately 30 hours, and less than 0.02s for the inference of one depth map on a GTX 1080 Ti GPU.

In the inference process the depth map was generated from the 3cGAN models by inputting the 2D image from the bronchoscopic camera. The bronchoscopic image and/or the depth map was then processed for airway detection using a combination of blob detection, thresholding, and peak detection (see e.g., FIG. 11A discussed below). Blob detection was performed on a depth map where 20% of the deepest area was thresholded, and the centroids of the resulting shapes were treated as potential points of advancement for the robot to bend and advance towards. Peak detection was performed as a secondary detection method to detect airways that may have been missed by the blob detection. Any peaks detected inside an existing detected blob were disregarded. Direction vector control command may be performed using the directed airways to decide to employ bending and/or insertion, and/or such information may be passed or transmitted to software to control the robot and to perform autonomous advancement.

As shown in FIGS. 1-9, one or more embodiments of the present disclosure may be a robotic bronchoscope using a robotic catheter and actuator unit, a robotic arm, and/or a control software or a User Interface. Indeed, one or more robotic bronchoscopes may use any of the subject features individually or in combination.

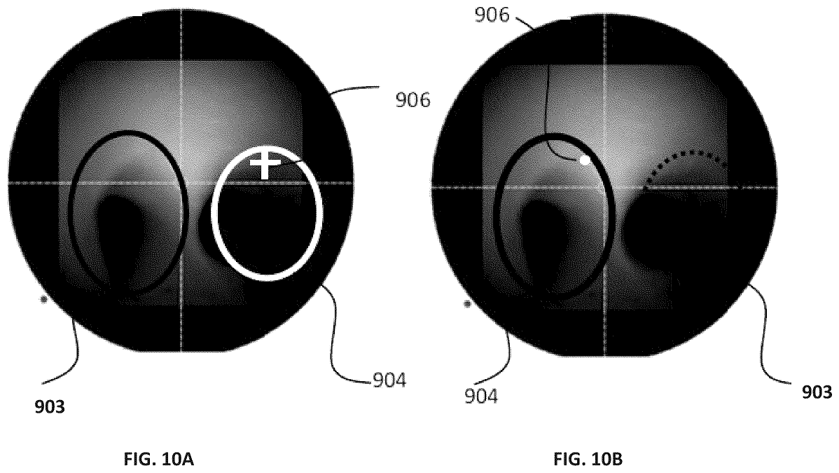

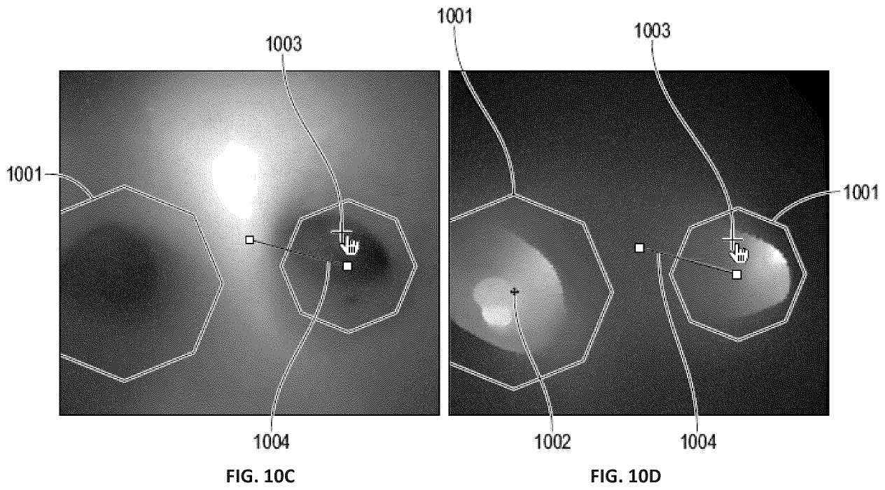

In one or more embodiments, depth estimation may be performed from bronchoscopic images and with airway detection (see e.g., FIGS. 10C-10D). Indeed, one or more embodiments of a bronchoscope (and/or a processor or computer in use therewith) may use a bronchoscopic image with detected airways and an estimated depth map (or depth estimation) with or using detected airways. A pixel of a set or predetermined color (e.g., red or any other desired color) 1002 represents a center of the detected airway. A cross or plus sign (+) 1003 may also be of any set or predetermined color (e.g., green or any other desired color), and the cross 1003 may represent the desired direction determined or set by a user (e.g., using a drag and drop feature, using a touch screen feature, entering a manual command, etc.) and/or by one or more processors (see e.g., any of the processors discussed herein). The line or segment 1004 (which may also be of any set or predetermined color, such as, but not limited to, blue) may be the direction vector between the center of the image/depth map and the center of the detected blob in closer proximity to the cross or plus sign 1003.

In the inference process the depth map was generated from the 3cGAN models by inputting the 2D image from the bronchoscopic camera. The depth map was then processed for airway detection using a combination of blob detection (see e.g., T. Kato, F. King, K. Takagi, N. Hata, IEEE/ASME Transactions on Mechatronics pp. 1-1 (2020), the disclosure of which is incorporated by reference herein in its entirety), thresholding, and peak detection (see e.g., F. Masaki, F. King, T. Kato, H. Tsukada, Y. Colson, and N. Hata, IEEE Transactions on Biomedical Engineering, vol. 68, no. 12, pp. 3534-3542 (2021), the disclosure of which is incorporated by reference herein in its entirety) (see e.g., FIGS. 10C-10D and related discussion herein). Blob detection was performed on a depth map where 20% of the deepest area was thresholded, and the centroids of the resulting shapes were treated as potential points of advancement for the robot to bend and advance towards. Peak detection (see e.g., F. Masaki, F. King, T. Kato, H. Tsukada, Y. Colson, and N. Hata, IEEE Transactions on Biomedical Engineering, vol. 68, no. 12, pp. 3534-3542 (2021)) was performed as a secondary detection method to detect airways that may have been missed by the blob detection. Any peaks detected inside an existing detected blob were disregarded.

In FIGS. 10A, the cursor 906 has been moved into path 2 (904), having a white circular feature. This can be done, for example by user instruction to select this path is selected as the target path. For example, the user can use the handheld controller (e.g., a joystick) 105 to select or change the target path by clicking on or inside a lumen or on or inside an indication of the lumen/path candidate. In other embodiments, a touchscreen or a voice input is used. The system controller may show, on the display, an indication of which lumen was selected by the user. This may be done in one of a number of ways, such as by highlighting the selected lumen, including a feature such as a circle, crosshair, or similar indicator on the chosen lumen, including a feature on each of the lumen and having a a color change (black versus white circles, or any other color scheme or other indicator, such as red and green, black and green, bolded, highlighted, dashed) or other visual indicator to indicate the detected lumen.

In the example shown in FIG. 10A, the system controller 128 may change the path 1 or path 2 based on the user instruction. FIG. 10A is a selection of the right-most lumen. FIG. 10B is a selection of the left-most lumen. In FIG. 10B, the selection is indicated by a bolder circle for path 1/904 the target path (e.g., path 1, 904) and a dashed circle for the unselected path 902. Concurrent user instruction for the target path among the paths allows reflecting user's intention to the robotic catheter system during autonomous navigation effectively. Another optional feature is also shown in FIG. 10B, where the cursor 906 has changed from a crosshair to a circle. In the workflow leading up to the image shown in FIG. 10B, the user moved the cursor 906, which is optimized for the user to view both the cursor and the underlying image, until it was touching or inside the selected path 904, at which time, the cursor 906 is changed to a less obvious indicator, shown here as a small circle.

While the user may select or change the target path at any point in the planning step, as the autonomous system move into the control step, the user can optionally adjust the target path as described herein, or the prior target path can be used until the predetermined insertion depth or end point is reached. The need for additional selection of the target path can depend on the branching of the lumen network, where, for example, the user provides target path information as each branch within the airway becomes visible in the camera image.

While color is used in this example to indicate the selection of the target path, other colors or other indicators may be used as well, such as a bolder indicator, a flashing indicator, removal of the indicator around the non-selected path lumen, etc.

By having the user output device and displaying symbols for the paths and user instruction GUI with endoscope view in the user output device, user can form their intention intuitively and accurately with the visual information in one place. The dedicated user instruction GUI allows the user to select the target path immediately even when the paths are more than two.

By having the user output device and displaying symbols for the paths and differentiating the symbol for the target path from the other paths with endoscope view in the user output device, the user can form intention intuitively and accurately with the visual information in one place. Particularly, the differentiating the symbol for the target path achieve the user instruction with the minimal symbols without the dedicated user instruction GUI and allows the user to learn/understand how to read symbols with the minimal effort.

As shown in embodiments herein, circles (or ovals) are used as the symbol of the paths on the 2D interface, and the cursor provides a symbol for input of user instruction to the GUI. These GUIs have minimal obstacles for the endoscope view in the user output device. Also, selecting object with the cursor are very familiar maneuver from common computer operation, the user can easily learn how to use it.

By pausing the actuator and the linear translation stage during user's instruction, the system can reduce the risk where the user miss the target path in their interaction. Also, this gives users to think and judge the target path among the paths without pressurizing the user to make decisions in the short time.

By allowing the user to add a new target path if the user cannot find the target path among the existing paths and find the target path in the endoscope view, the plan generated by the system becomes more accurate with minimal effort of the user.

### Voice Input and Visualization

In some embodiments, the user input device is a voice input device. Since the autonomous system is driving the steerable catheter, full directional control of the catheter is unnecessary for autonomous driving. It can also be unwanted. A limited library of commands that the user can provide gives full control to the user to select which lumen is the correct one for the next navigation step, but prevents the user from, for example, trying to keep the steerable catheter in the center of the lumen as they would do with a manual catheter since this can be accomplished through the autonomous function. The limited library also simplifies the system.

Effective voice commands can be in the form of a limited library in the system and are, for the one system as provided herewith are (1) start, (2) stop, (3) center, (4) up, (5) down, (6) right, (7) left, and (8) back. Other systems may have more or fewer commands. Some systems will have a different selection of commands, depending on the use for the steerable catheter. In this embodiment, voice commands are classified as one of the effective commands and acted on as such. Verbalizations that are not classified as one of the effective commands are ignored when the sent command is not recognized as the effective commands. In some embodiments, the user or users train the system to recognize their particular enunciation of the effective voice commands. In other embodiments, the system is pre-set with the range of enunciations that are effective commands.

In some embodiments, the instructions are limited to the eight commands listed above or variants thereof. In other embodiments, the instructions are limited to less than or equal to 4, 6, 8, 10, 12, 14, 16, 18, 20, or 24 commands. The commands may all be limited to instructions for selecting a target path in a lumen.

Some embodiments provide autonomous navigation with voice command. When the user starts the autonomous navigation, the user sends a voice command, “start” (S1010) and “Autonomous navigation mode” is displayed on the main display 118. The autonomous navigation system detects airways in the camera view (S1020). The centers of detected airways are displayed as diamond mark (560) in the detected airways in the camera view.

In some embodiments, in order for the user to select an airway for the steerable catheter to move in, the user sends one of voice commands from the options of “center”, “up”, “down”, “right” and “left”. When the voice command is accepted by the system, the color of “x” mark on the selected location is changed from black to red and a triangle 570 is displayed on the selected mark.

The selected location stays at the same location until a different location is accepted to the system. As the default, the “x” mark on the center is set when the autonomous navigation mode is started.

The system sets the closest airway from the selected x mark as the airway to be aimed based on the distance between the selected x mark and each diamond mark in the detected airways (S1030).

By choosing the closest airway as an operator's intended airway from the “+” mark, which the operator moves with the voice commands, the operator can instruct the intended airway intuitively and accurately. The operator can always clearly confirm the distance between the “+” mark and the intended airway on the display and easily understand which airway option the autonomous system will choose on the display. Therefore, this transparency gives the operator predictable system behavior and operation confidence during autonomous operation.

Also, since the position options of “+” mark include a limited number, the operator can determine the next position option easily and quickly.

Moreover, with this method, the autonomous system always has at least one intended airway until there is at least one airway candidate. This feature avoids the situation without the intended airway and make the system behavior robust.

The user can stop the autonomous navigation any time by sending a voice command, “stop”, and can start the manual navigation using a handheld controller 105 to control the steerable catheter and the linear translational stage. When the user takes over the control, “Manual navigation mode” is displayed on the main display 118. The user can restart the autonomous navigation when needed by sending a voice command, “start”. Alternatively, the user can stop the autonomous navigation by, for example, an interaction with the handheld controller.

While the robotic platform is bending the steerable catheter and moving the linear translational stage forward, all input signals to the robotic platform are recorded in the data storage memory HDD 188 regardless of navigation modes. When the user needs to retract the robotic platform, the user sends a voice command, “back”, then the robotic platform inversely applies the recorded input signals taken during insertion to the steerable catheter and the linear translational stage. During retraction, the system displays “back” on the display.

(Backup and alternative systems) Instead of sending voice commands, the user can send the commands using other input devices including a number pad or a general computer keyboard. The commands can be assigned as numbers on the number pad or other letters on the keyboard. The other input devices may be used along with or instead of voice commands. In some embodiments, the input device is has a limited number of keys/buttons that can be pressed by the user. For example, a numerical keypad is used where 2, 4, 6, and 8 are the four directions and 5 is center. The additional numbers on the keypad may be without function, or they may provide an angled movement.

### Navigation/Driving

The integrated control using first-person view, grants physicians the capability to guide the distal section's motion via visual feedback from the robotic bronchoscope. For forward motion/navigation, users may determine only the lateral and vertical movements of the third (e.g., most distal) section, along with the general advancement or retraction of the robotic bronchoscope. The user's control of the third section may be performed using the computer mouse and drag and drop a cross or plus sign 1003 to the desired direction as shown in FIGS. 10C-10D, FIG. 11A, and/or FIG. 11B. A voice control may also be implemented additionally or alternatively to the mouse-operated cross or plus sign 1003. For example, an operator or user may select an airway for the robotic bronchoscope to aim using voice recognition algorithm (VoiceBot, Fortress, Ontario, Canada) via a headset (J100 Pro, Jeeco, Shenzhen, China). The options acceptable as input commands to control the robotic bronchoscope were the four cardinal directions (up, down, left, right, and center) and start/stop. For example, when the voice recognition algorithm accepted “up”, a cross 1003 was shown on top of the endoscopic camera view. Then, the system automatically selected the closest airway to the mark out of the airways detected by the trained 3cGAN model, and sent commands to the robotic catheter to bend the catheter toward the airway (see FIG. 11B, which shows an example of the camera view in a case where voice recognition algorithm accepted “up”. The cross 1003 indicated in which direction was being selected and the line or segment 1004 showed the expected trajectory of the catheter).

Additionally or alternatively, any feature of the present disclosure may be used with features, including, but not limited to, training feature(s), autonomous navigation feature(s), artificial intelligence feature(s), etc., as discussed in International Patent Application No. PCT/US2024/037935, filed Jul. 12, 2024, the disclosure of which is incorporated by reference herein in its entirety; International Patent Application No. PCT/US2024/037924, the disclosure of which is incorporated by reference herein in its entirety; and International Patent Application No. PCT/US2024/037930, the disclosure of which is incorporated by reference herein in its entirety.

### Control

For specifying the robot's movement direction, the target airway is identified based on its center proximity to the user-set marker visible as the cross or cross/plus sign 1003 in one or more embodiments as shown in FIGS. 10C-10D and/or FIG. 11B (the cross may be any set or predetermined color, e.g., green or other chosen color). In one or more embodiments, a direction vector may be computed from the center of the depth map to the center of this target detected airway. In one or more embodiments, the vector may inform a virtual gamepad controller (or other type of controller) and/or one or more processors, instigating or being responsible for the bending of the bronchoscopic tip. In one or more embodiments, a desired target airway may be selected using voice control, a click/touch on an image, an instruction being sent directly to the controller/processor, etc. In one or more embodiments, the robot may advance in a straight line if this direction vector's magnitude is less than 30% of the camera view's width, which is called linear stage engagement (LSE). In one or more embodiments, the process may repeat for each image frame received from the bronchoscopic camera without influence from previous frames. In one or more embodiments, the bronchoscopic robot may maintain a set or predetermined/calculated linear speed (e.g., of 2 mm/s) and a set or predetermined/calculated bending speed (e.g., of 15 deg/s). One or more of such features may provide at least one or more of the following benefits/advantages: (i) having direct communication reduces latency and increases responsiveness; (ii) having an ability to move the stage backwards and to command bending of other catheter section(s) (e.g., simultaneously or separately); (iii) having more precision over when/where/how certain motion commands are applied; (iv) having improved smoothness of motion and a reduction in navigation time by bending catheter section(s) and moving a stage simultaneously; (v) addressing situations where a catheter may not be able to align with an airway by employing retracting in one or more embodiments; (vi) reducing or minimizing situations where control command(s) is/are sent based on an incorrect airway; (vii) identifying situations which are pertinent to autonomous control, such as breathing motion and the entering of a new generation; and/or (viii) improving alignment timing and accuracy by sending a larger motion in a case where a target is farther away and by sending a more refined motion when a target is closer.

One or more embodiments may employ a command set and communication method(s) or technique(s). The location of the target within the 2D image may determine the control commands to be relayed, ultimately, to the one or more controllers which drive the movement of the catheter. These control commands may be represented as gamepad commands, serial commands, or any other form of communication/message in which the controllers may convert to catheter movements. These movements may be one or more of stage insertion/retraction, catheter tip bending, catheter tip displacement, or other catheter section bending/displacement/state change. As aforementioned, direct communication reduces latency and increases responsiveness, and an ability to move the stage backwards and to command bending of other catheter section(s) may be employed.

One or more embodiments may use command thresholding. The direction vector's magnitude may be included in the determination of which control commands to send. In some embodiments, there may be a distinct threshold (e.g., 30% of the camera view's width) or threshold range wherein a bending command may be utilized in a case where the magnitude falls upon one side of the threshold or threshold range, and a linear stage insertion command may be utilized in a case where the magnitude falls on the other side of the threshold or threshold range. As aforementioned, one or more embodiments may: introduce more precision over when/where/how certain motion commands are applied, improve smoothness of motion and reduce navigation time by bending and moving a stage simultaneously, and address situations where a catheter may not be able to align with an airway by using retraction.

In one or more embodiments, there might also be additional thresholds upon which no commands are sent when the magnitude falls upon a particular side. In other embodiments, a linear stage retraction command can be designated when the magnitude is within a particular threshold range. In this case, if the target is far to the edge of the image, retraction might help bring it closer towards the center of the field of view. A reverse FTL (rFTL) algorithm (which may operate as the FTL algorithm working in reverse or to move a catheter in reverse) activated during retraction may also help with the control as the rFTL technique(s) may operate to re-orient the catheter sections in a manner in which there is less impedance in the bending required to align with the target airway.

Additionally, any command may reside within any threshold level. Also, more than one command may be utilized within the same threshold area. The size of the thresholds and the corresponding commands may vary either through user input (e.g., manually), automatically based on one or more conditions (like the control mode, catheter pose(s), insertion depth, direction vectors angle, etc.), or a combination of one or more of the same.

In other embodiments, there might be multiple threshold frames which can be overlapping. For example, Insertion can have a maximum threshold of 35% of the camera views width, while Bending can have a minimum threshold of 25% of the camera views width. In this scenario, both bending and insertion will be commanded when the magnitude falls within 25% and 35% of the camera views width. Under these conditions, the utilize of FTL will assist with the bending motion as the middle and proximal sections will also begin to guide the tip towards the target airway.

In one or more embodiments, target history technique(s) may be used. The process may repeat for each image frame received from the bronchoscopic camera with or without influence from previous one or more frames. In one or more embodiments, the difference in location of the target airway may affect the control determination. For example, in a case where there is a very large displacement of the target airway (and/or it is very close to an unselected airway in the earlier frame), it may be determined to be a misdetection, and no movement may be commanded until the next frame. Similarly, in a case where the magnitude of the target airways direction vector increases, it may indicate breathing motion, and the control output may be muted or subdued until the magnitude and/or the breathing motion returns to an acceptable range. In one or more embodiments, a target airway near the center of the camera image may no longer be found in later frames, and it might be assumed that a new branch has been entered. Such a situation may be informed to a user, and the situation and information may affect the control determination. As aforementioned, one or more embodiments may reduce situations where the control commands are sent based on an incorrect airway and may identify situations which are pertinent to autonomous control, such as breathing motion and the entering of a new generation (e.g., of airway(s)). Data (e.g., 3D) data may be combined as a catheter progresses to generate a bigger, more accurate map of an entire pathway in one or more embodiments.

The threshold levels which determine how the direction vectors change affect the controls, as well as which of the corresponding controls represented by these thresholds, may be fixed/predetermined, adjusted manually from user input, adjusted automatically based on prior changes or other one or more conditions, or a combination of such features.

One or more embodiments of the present disclosure may use command speed and magnitude features or techniques. The speed and magnitudes of the insertion, retraction, and bending commands relayed to the controller(s) or one or more processors may be fixed/predetermined, adjusted by user input, calculated/parametric, or any combination thereof. In one or more embodiments, the speed and/or magnitude of the motion commands may depend on the direction vectors magnitude. Similarly, in one or more embodiments, these command levels may be proportional to the position of the target airway within its corresponding threshold or threshold range. In such scenarios, the threshold may be considered to represent a gradient of speed and/or magnitude of motion commands. The gradient may be discrete or continuous, and the slope or range size may follow a mathematical formula (e.g., linear, logarithmic, exponential, any other predetermined formula, any other formula selected by a user, etc.), may follow a parameterized function based on one or more control conditions, may be user defined, or may be any combination thereof. This gradient may also have ranges within where the slope/size calculation differs from other ranges. Again, the determination of the size/position of these ranges, as well as the corresponding slope/size calculations, may be a generic formula, may be parameterized based on one or more robot conditions, may be user defined, or may be any combination of such features. As aforementioned, one or more embodiments may improve alignment timing and accuracy by sending a larger motion (e.g., a command for same) in a case where a target is further away (e.g., over a distance threshold, equal to or over a distance threshold, etc.) and by sending a more refined motion (e.g., a command for same) in a case where the target is closer (e.g., under a distance threshold, under or equal to a distance threshold, etc.).

In one or more embodiments, all possible command which controllers (e.g., a joystick, a gamepad controller, one or more processors, etc.) accept may be utilized. In one or more embodiments, commands may be combined and more control may exist over when/how/where each individual command is activated/deactivated. One or more embodiments may use past states for error detection and control decisions. One or more embodiments may react to a target location within an image or frame (e.g., within a camera frame or image) by sending or commanding a predetermined, set, or appropriate amount of motion.

Simultaneously, in one or more embodiments, the movements of the initial two sections (first and second sections) may be managed by the FTL motion algorithm, based on the movement history of the third section. During retraction, the reverse FTL motion algorithm may control all three sections, leveraging the combined movement history of all sections recorded during the advancement phase, allowing users to retract the robotic bronchoscope whenever necessary. By applying FTL, a most distal segment may be actively controlled with forward kinematic values, while a middle segment and another middle or proximal segment (e.g., one or more following sections) of a steerable catheter or continuum robot move at a first position in the same way as the distal section moved at the first position or a second position near the first position. The FTL algorithm may be used in addition to the robotic control features of the present disclosure. For example, by applying the FTL algorithm, the middle section and the proximal section (e.g., following sections) of a continuum robot may move at a first position (or other state) in the same or similar way as the distal section moved at the first position (or other state) or a second position (or state) near the first position (or state) (e.g., during insertion of the continuum robot/catheter, by using the navigation, movement, and/or control feature(s) of the present disclosure, etc.). Similarly, the middle section and the distal section of the continuum robot may move at a first position or state in the same/similar/approximately similar way as the proximal section moved at the first position or state or a second position or state near the first position (e.g., during removal of the continuum robot/catheter). Additionally or alternatively, the continuum robot/catheter may be removed by automatically and/or manually moving along the same or similar, or approximately same or similar, path that the continuum robot/catheter used to enter a target (e.g., a body of a patient, an object, a specimen (e.g., tissue), etc.) using the FTL algorithm, including, but not limited to, using FTL with the one or more control, depth map-driven autonomous advancement, or other technique(s) discussed herein. Other FTL features may be used with the one or more features of the present disclosure.

At least one embodiment of the pipeline of a workflow for one or more embodiments is shown in FIG. 11A. For example, one or more embodiments may receive/obtain one or more bronchoscopic images (which may be input into a 3cGAN or any other AI-related architecture/structure for processing) such that a network (e.g., a neural network, a 3cGAN, a GAN, a convolutional neural network, any other AI architecture/structure, etc.) and/or one or more processors may estimate a depth map from the one or more bronchoscopic images. An airway detection algorithm or process may identify the one or more airways in the bronchoscopic image(s) and/or in the depth map (e.g., such as, but not limited to, using thresholding, blob detection, peak detection, and/or any other process for identifying one or more airways as discussed herein and/or as may be set by a user and/or one or more processors, etc.). As aforementioned, the pixel 1002 may represent a center of a detected airway and the cross or plus sign 1003 may represent the desired direction determined by the user (e.g., moved using a drag and drop feature, using a touch screen feature, entering a manual command, etc.) and/or by one or more processors (see e.g., any of the processors discussed herein). The line or segment 1004 (which may also be of any set or predetermined color, such as, but not limited to, blue) may be the direction vector between the center of the image/depth map and the center of the detected blob closer or closest in proximity to the cross or plus sign 1003. The direction vector control command may decide between bending and insertion. The direction vector may then be sent to the robot's control software by a virtual gamepad (or other controller or processor) which may initiate the autonomous advancement. As shown in FIGS. 10C-10D and/or FIG. 11A, at least one embodiment may have a network estimate a depth map from a bronchoscopic image, and the airway detection algorithm(s) may identify the airways. The pixel 1002, the cross or plus sign 1003, and the line or segment 1004 may be employed in the same or similar fashion such that discussion of the subject features shown in FIGS. 10C-10D, FIG. 11A and FIG. 11B will not be repeated.

Characteristics of models and scans for at least one study performed is shown in Table 1 below:

TABLE 1
Characteristics of Phantom and Ex vivo models and scans

| Model Target | | # of | CT dimensions | CT spacing |
|---|---|---|---|---|
| Generations | | targets | [mm] | [mm] |
| Phantom | 4-10 | 75 | 512 × 512 × 412 | 0.689 × 0.689 × 0.6 |
| Ex-vivo #1 | 3-15 | 52 | 512 × 512 × 661 | 0.939 × 0.939 × 0.6 |
| Ex-vivo #2 | 3-12 | 41 | 512 × 512 × 574 | 0.854 × 0.854 × 0.6 |

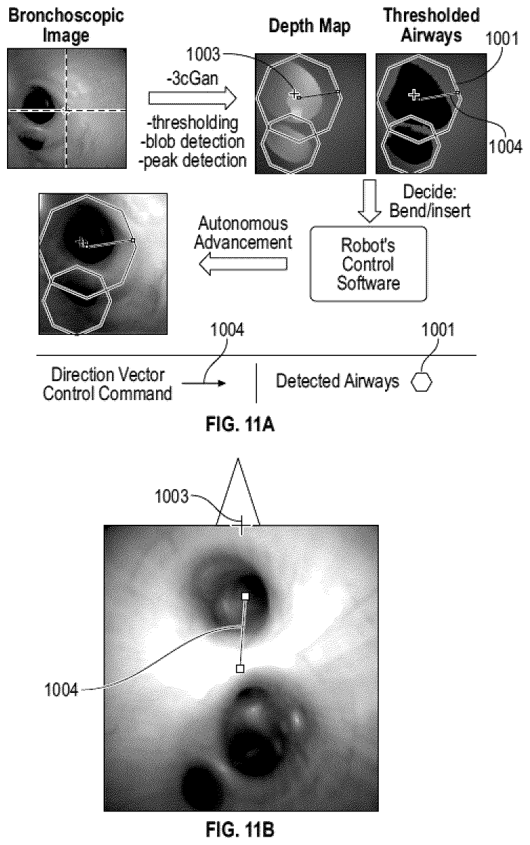

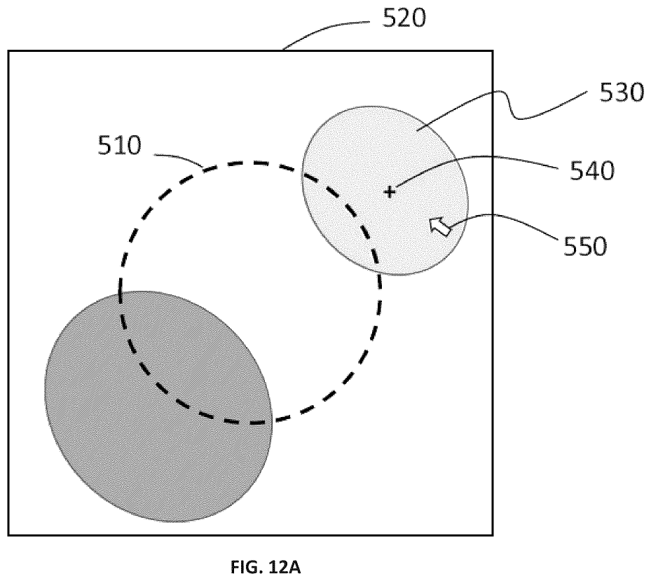

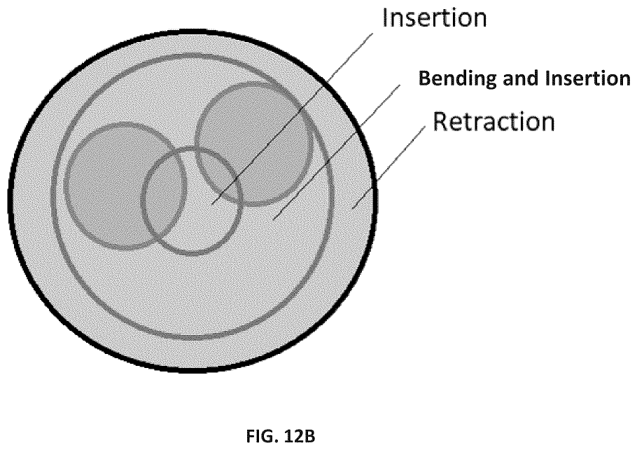

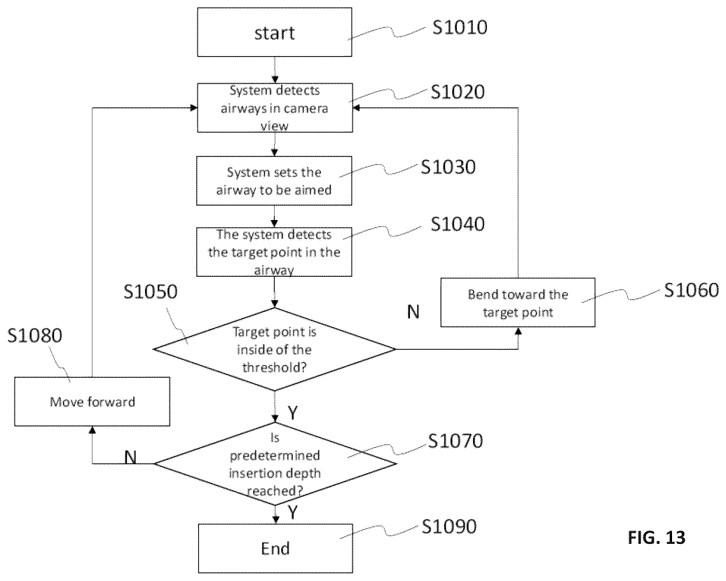

FIG. 12A shows the typical camera image during the autonomous navigation, and FIG. 12B shows at least one embodiment example of an image showing different zones and how each relate to insertion, bending, and retraction. As shown in the middle of FIG. 12B, the bending area may be an area where insertion is also performed. FIG. 13 shows a flowchart of at least one exemplary autonomous navigation process(es) or technique(d).

In one or more embodiments, the autonomous navigation system receives an image from the camera and defines a position point in the image. The position point can be used as a proxy to the position of the distal tip of the continuum robot. The position point may be the center of the camera view. In other embodiments, the position point is offset from the center of the camera view based on, for example, a known offset of the camera within the continuum robot.

The autonomous navigation system compares the distance between the position point (e.g., the center of the camera view) and the target point with the threshold (S1050). If the distance between the position point and the target point is longer than the threshold, the robotic platform bends the steerable catheter toward the target point (S1060) until the distance between the position point and the target point is smaller than the threshold. If the distance between the position point view and the target point is smaller than the threshold, the robotic platform moves the linear translational stage forward (S1080).

In FIG. 13, the system detects the airways in the camera view (S1020). This method and system are particularly described above, and include using a depth map produced by processing one or more images obtained from the camera, fitting the lumen or lumens (e.g., airways) using an algorithm such as a circle fit, peak detection algorithm or similar, where, in instances of the camera image not able to perform the algorithm to find one or more lumen, using the deepest point. Next, the system sets the airway to be aimed (S1030). This provides a target path. This can be done by user interaction as discussed herein or through an automated process.

As discussed above in reference to FIG. 6, the route of the continuum robot to reach the target is determined and (in step s605), the generated model and the decided route on the model may be stored. In this target path, a plurality of target points are used to navigate the continuum robot along the target path. The target points in the lumen (e.g., airway) define for the target path (S1040). The target point may be, for example, the center of the circle that was used to fit to the airway. In FIG. 12A, the target point is shown as a “+” mark 540 and an arrow 580 connecting the “+” mark and the center of the camera view (S1040).

Then, the autonomous system must aim the distal end of the steerable catheter towards the target point and move the steerable catheter forward, towards the target point (S1050). For this step, a threshold is set. If the target point is inside of the threshold, the steerable catheter is advanced, increasing the insertion depth (S1070). If the target point is not inside of the threshold, then the distal end of the steerable catheter is bent towards the target point (S1060). After the steerable catheter is bent further, the controller must re-assess whether the target point is inside of the threshold (S1050). If the steerable catheter has not yet reached the region of interest, or as at the predetermined insertion depth or end point, the steerable catheter will move forward S1080). Then these two steps of bending the continuum robot towards the target point and moving the continuum robot forward along the target path are repeated, with new images taken periodically to be used at each iteration to determine if the continuum robot is to move forward or to bend to more closely point to the target point. If the steerable catheter has reached the region of interest, or as at the predetermined insertion depth or end point, the automated procedure will end (S1090).

Of note, in one or more processes, after a particular iteration of moving forward (S1080) or bending toward the target point (S1060), the system then returns to detecting the lumen (e.g., airway) in the camera view. Thus, each iteration can be performed with a new and separate image taken by the camera. Knowledge of locations within the last image as well as knowledge obtained from prior data, such as from a pre-operative CT image, are not required. Therefore, robust registration algorithm(s) are not required to perform this automated driving function. This can be particularly advantageous in multiple situations and will not be effected by breathing as much as many attempts at autonomous navigation as seen in the literature.

For a bronchoscopic procedure, various parameters of the robotic platform including the frame rate of the camera, the bending speed of the steerable catheter, the speed of linear translational stage 110, and the predetermined insertion depth or end point of the linear translational stage are set. They each independently may be preset (e.g., a standard base value) or set by the user for the procedure. If set by the user, it may be based on the user's preference, or based on specifies of the patient. The parameters may be calculated or be obtained from a look-up table. The predetermined insertion depth or end point may be estimated as the distance from the carina to the region of interest based on, for example, one or more preprocedural CT images. These parameters may be set before the start of the automated motion portion of the procedure, such as when the steerable catheter 104 reaches the carina.

A threshold is used during autonomous function to decide whether to bend the steerable catheter to optimize the direction and/or angle of the tip or to move forward using the linear translational stage. The threshold 510 may be a constant and can be defined based on the dimensions of the camera view. The threshold relates to the distance from the center of the camera view, (e.g., the center of the a dotted circle 510 in a camera view 520, which is the center of the distal end of the steerable catheter) to the center of the airway 530 that has been selected as the target path (the target point 540). The threshold value is visualized in FIG. 12A as a dotted circle 510, however, it can alternatively be configured as a vector. The vector represents the direction of movement on the image plane of the image from the camera and depth map images, where the magnitude of the vector is the threshold value. The distance to be set as the threshold may be decided based on, for example, data from a lung phantom model. Alternatively, the threshold may be based on a library of threshold data. In some embodiments, the threshold set to 10%, 15%, 20%, 25%, 30%, 35%, or 40% or a value therebetween of the camera view dimension (i.e., the distance of a diagonal line across the camera view). In some embodiments, the threshold may be set to between 25% and 35%, or around 30%.

In some embodiments, when the user starts the autonomous navigation, an indicator of the navigation mode being used, such as displaying “Autonomous navigation mode” on the main display 118. The autonomous navigation system detects airways in the camera view (S1020). The user places a mouse pointer 550 on the airway to be aimed 530 in the camera view for the autonomous navigation system to set the airway to be aimed (S1030). The autonomous navigation system detects the target point 540 in the detected airway as described above (S1040), then the autonomous navigation system compares the distance between the center of the camera view and the target point with the threshold (S1050). If the distance between the center of the camera view and the target point is longer than the threshold, the robotic platform bends the steerable catheter toward the target point (S1060) until the distance between the center of the camera view and the target point is smaller than the threshold. If the distance between the center of the camera view and the target point is smaller than the threshold, the robotic platform moves the linear translational stage forward (S1080).

In some embodiments, the user has the ability to stop the autonomous navigation any time by pushing a button on the handheld controller 105 and can start the manual navigation to control the steerable catheter and the linear translational stage by the handheld controller. When the user takes over the control, an indicator of this control is provided, such as a display of “Manual navigation mode” on the main display 118. The user can restart the autonomous navigation when needed by, for example, pushing a button on the handheld controller 105.

When the steerable catheter reaches a position close to the region of interest (e.g., tumorous tissue), the user has the option to switch the navigation mode to the manual navigation and, for example, deploy a biopsy tool toward the region of interest to take a sample through the working tool access port.

A Return to the Carina function may be included with the autonomous driving catheter and system. While the robotic platform is bending the steerable catheter and moving the linear translational stage forward, all input signals to the robotic platform may be recorded in the data storage memory HDD 150 (see also, hard disk 1204, SSD 1207, or any other data storage known to those skilled in the art, etc. that may be used to record data) regardless of navigation modes. When the user indicates a start of the Return to Carina function (e.g., hitting the appropriate button on the handheld controller 105), the robotic platform inversely applies the recorded input signal taken during insertion to the steerable catheter and the linear translational stage.

In some embodiments, an insertion depth may be set before driving the steerable catheter. When the linear translational stage reaches the predetermined insertion depth or end point, the autonomous navigation system can be instructed to stop bending the steerable catheter and moving the linear translational stage forward (S1070). The robotic platform then switches the mode from the autonomous navigation to the manual navigation. This allows the user to start interacting with the region of interest (e.g., take a biopsy) or to provide additional adjustments to the location or orientation of the steerable catheter.

In some embodiments, the frame rate is set for safe movement. The steps from S1020 to S1080 in FIG. 13 can be conducted at every single frame of camera image. Thus, depending on the capability of the Central Processing Unit 182, the maximum frame rate of the camera image that can be handled for the autonomous navigation is decided. Then based on the frame rate and the acceptable risk during bronchoscopy, the speed of bending the steerable catheter and the speed of moving the linear translational stage are decided. If it is important to move the steerable catheter based on the images when the steerable catheter is moving faster than can be ‘seen’ by the images from the camera. In an example where the maximum frame rate is 10 frames per second (fps) and the acceptable amount of the airway pushed by the steerable catheter is 0.5 mm, the speed of the linear translational stage may be set less than 5 [mm/see]. Other frame rates and risk factors will suggest different speeds.

### Variable Speed

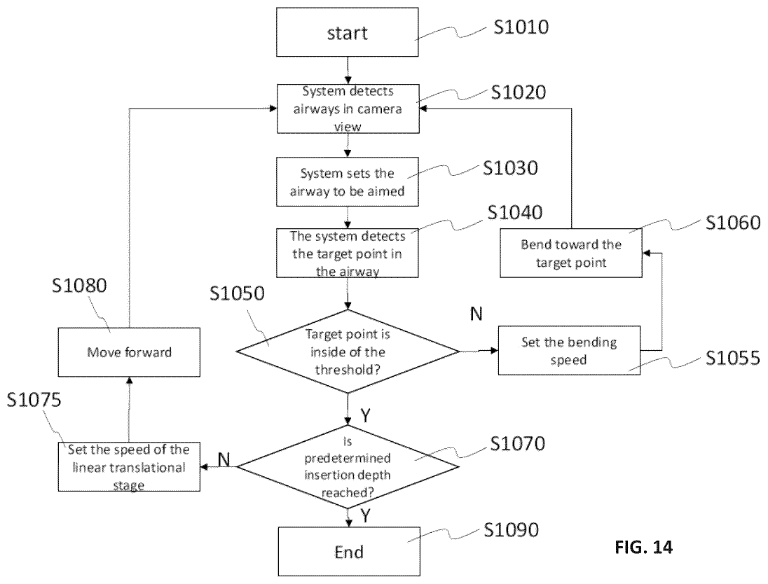

FIG. 14 shows a flowchart for at least one embodiment of how to set the speed of bending the steerable catheter and the speed of moving the linear translational stage based on the target point in the detected airway to be aimed.

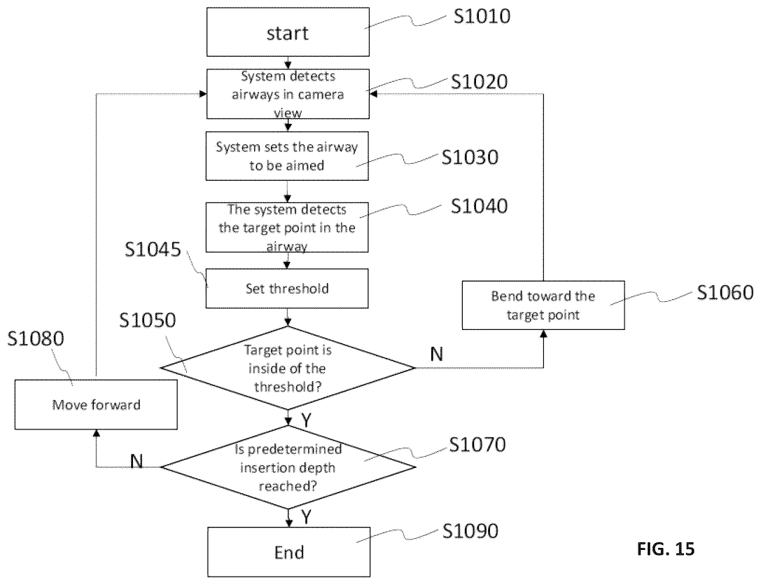

FIG. 15 shows an exemplary flowchart to adjust the threshold based on the location of the steerable catheter in the lung. In this workflow, the system detects the airway or airways in the camera view (S1020). The system then sets an airway to be aimed towards (S1030). Information as to which airway will be aimed may come from user input or from the controller. Next, the system detects the target point in the airway (S1040). A threshold is set (S1045) and it is determined whether the target point is inside of the threshold (S1050). In this case, as the position point is set as the center of the image, so the target point is inside of the threshold (S1050) if the target point is sufficiently close to the current position of the steerable catheter tip. If the target point is not inside the threshold, the tip of the steerable catheter in the lung is bent towards the target point (S1060). This bend may be a set amount, it may be to the approximate location of the target point, etc. After the catheter is bent, the system detects the airway in the camera view again (S1020) and the workflow repeats. If, in step S1050, the target point is inside of the threshold, it is determined whether the predetermined insertion depth or end point is reached (S1070). If this depth is not yet reached, the steerable catheter is moved forward in the airway (S1080). Once a new location is reached (which may be a set insertion distance, a distance set by the user, etc.), the system detects the airways in the camera view (S1020) and the workflow repeats. If, in step S1070, the predetermined insertion depth or end point is reached, the workflow is ended (S1090).

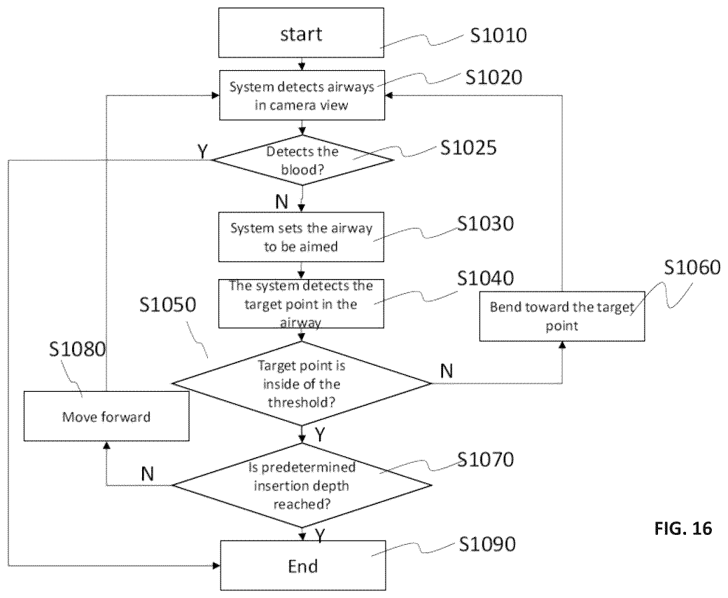

FIG. 16 shows a flowchart with an exemplary method to abort the autonomous navigation when the blood is detected in the camera view. The criterion to abort bronchoscopy may be defined as the ratio of the number of pixels indicating the blood divided by the total number of pixels in a camera image. In this embodiment, an imaging processing library, e.g., OpenCV, is used to count the number of red pixels in a RGB camera view. If the ratio of the number of pixels indicating the blood divided by the total number of pixels in a camera image exceeds the predetermined ratio, the autonomous navigation is aborted. Similar to the blood, the mucous in the airway can be detected using an imaging processing library. For detecting mucus, the number of yellow pixels in a RGB camera view can be used. The steerable catheter can be automatically stopped during bronchoscopy when an emergency situation is detected.

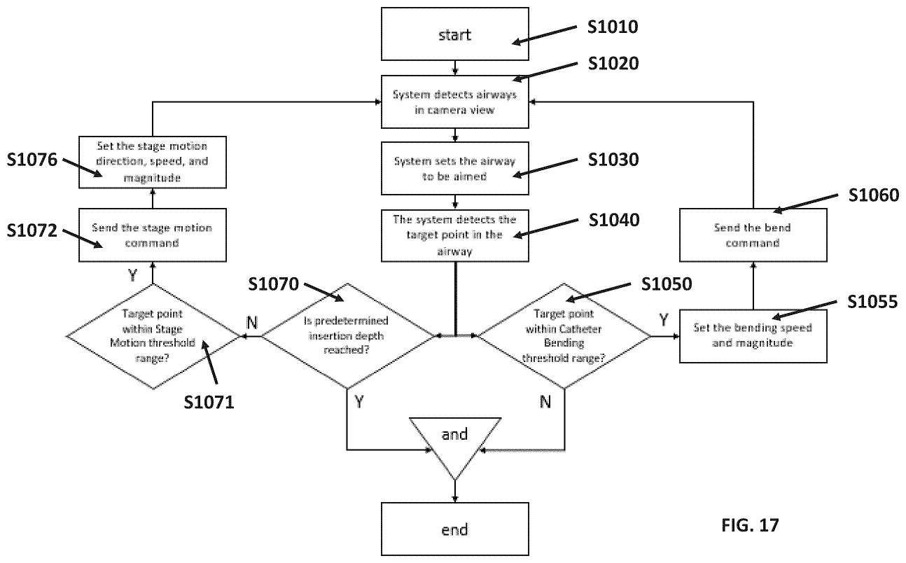

FIG. 17 shows a flowchart of at least one embodiment for determining simultaneously whether to bend one or more catheter section(s) and to move a translational stage. Description(s) for like numbered steps will not be repeated. As shown in FIG. 17, the flowchart ends in a case where the predetermined insertion depth or end point is reached (“Yes” in step S1070) and a target point is not within a catheter bending threshold range (“No” in step S1050). In a case where the predetermined insertion depth or end point has been reached and in a case where the target point is not within the catheter bending threshold range, then the driving/navigation and/or the planning is completed and/or a sample is taken using the catheter. If “Yes” for step S1050, then a bending speed and magnitude are set in step S1055, and the bend command is sent in step S1060. If “No” in step S1070, then another determination is made in step S1071 as to whether the target point is within a stage motion threshold range. If “Yes” in Step S1071, then the method proceeds to step S1072 to send a stage motion command and then to step S1076 to set the stage motion direction, speed, and magnitude. If “No” in step S1071, the process either waits until the target point is within the stage motion threshold range or intervention is used (e.g., manually via user input, automatically via processor intervention using stored or set data, etc.).

### Materials

### Patient-Derived Phantoms and Ex-Vivo Specimens/Animal Model

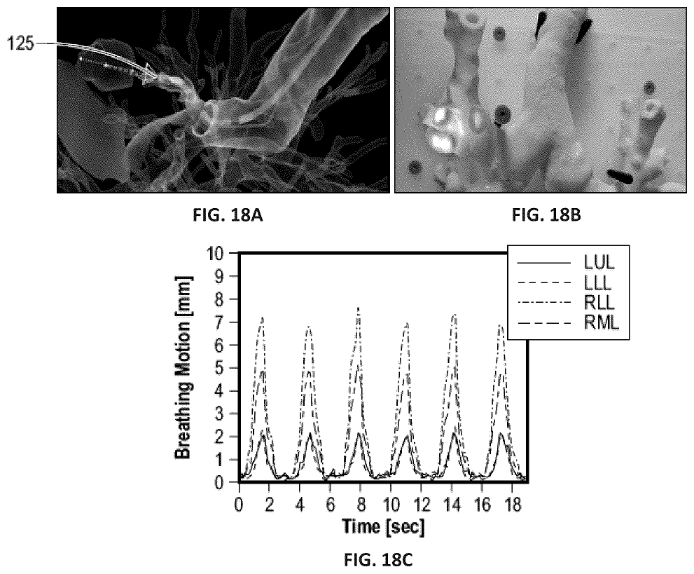

Imaging and airway models: The experiments utilized a chest CT scan from a patient who underwent a robotic-assisted bronchoscopic biopsy to develop an airway phantom (see FIG. 18B), under the IRB approval #2020P001835. FIG. 18B shows a robotic bronchoscope in the phantom having reached the location corresponding to the location of the lesion in the patient's lung, using the proposed supervised-autonomous navigation and/or navigation planning. The 62-year-old male patient presented with a nodule measuring 21×21×16 [mm] in the right upper lobe (RUL). The procedure was smoothly conducted using the Ion Endoluminal System (Intuitive Surgical, Inc., Sunnyvale, CA), with successful lesion access (see FIG. 18A showing the view of the navigation screen with the lesion reached in the clinical phase). FIGS. 18A-18B illustrate a navigation screen for a clinical target location 125 in or at a lesion reached by autonomous driving and a robotic bronchoscope in a phantom having reached the location corresponding to the location of the lesion using one or more navigation features, respectively. Various procedures were performed at the lesion's location, including bronchoalveolar lavage, transbronchial needle aspiration, brushing, and transbronchial lung biopsy. The procedure progressed without immediate complications. The inventors, via the experiment, aimed to ascertain whether the proposed autonomous driving method(s) would achieve the same clinical target (which the experiment confirmed that such method(s) would achieve the same clinical target). Thus, one target in the phantom replicated the lesion's location in the patient's lung. Airway segmentation of the chest CT scan mentioned above was performed using ‘Thresholding’ and ‘Grow from Seeds’ techniques within 3D Slicer software. A physical/tangible mold replica of the walls of the segmented airways was created using 3D printing in ABS plastic. The printed mold was later filled to produce the Patient Device Phantom using a silicone rubber compound, which was left to cure before being removed from the mold.

The inventors, via the experiment, also validated the method features on two ex-vivo porcine lungs with and without breathing motion simulation. A human Breathing motion was simulated using an AMBU bag with a 2-second interval between the inspiration phases.

### Target and Geometrical Path Analysis

CT scans of the phantom and both ex-vivo lungs have been performed (see Table 1) and airways were segmented using ‘Thresholding’ and ‘Grow from Seeds’ techniques in 3D Slicer.

The target locations were determined as the airways with a diameter constraint imposed to limit movement of the robotic bronchoscope. The phantom contained 75 targets, where ex-vivo lung #1 had 52 targets, and ex-vivo lung #2 had 41 targets. The targets were positioned across all airways. This resulted in generating a total number of 168 advancement paths and 1163 branching points without breathing simulation (phantom plus ex-vivo scenarios), and 93 advancement paths and 675 branching points with breathing motion simulation (BM) (ex-vivo) (see Table 1). Each of the phantoms and specimens contained target locations in the lobes.

Each target location was marked in the segmented model, and the Local Curvature (LC) and Plane Rotation (PR) were generated along the path from the trachea to the target location and were computed according to the methodology described by Naito et al. (M. Naito, F. Masaki, R. Lisk, H. Tsukada, and N. Hata, International Journal of Computer Assisted Radiology and Surgery, vol. 18, no. 2, pp. 247-255 (2023), the disclosure of which is incorporated by reference herein in its entirety). LC was computed using the Menger curvature, which defines curvature as the inverse of the radius of the circle passing through three points in n-dimensional Euclidean space. To calculate the local curvature at a given point along the centerline, the Menger curvature was determined using the point itself, the fifteen preceding points, and the fifteen subsequent points, encompassing approximately 5 mm along the centerline. LC is expressed in [mm⁻¹]. PR measures the angle of rotation of the airway branch on a plane, independent of its angle relative to the trachea. This metric is based on the concept that maneuvering the bronchoscope outside the current plane of motion increases the difficulty of advancement. To assess this, the given vector was compared to the current plane of motion of the bronchoscope. The plane of motion was initially determined by two vectors in the trachea, establishing a plane that intersects the trachea laterally (on the left-right plane of the human body). If the centerline surpassed a threshold of 0.75 [rad](42 [deg]) for more than a hundred consecutive points, a new plane was defined. This approach allowed for multiple changes in the plane of motion along one centerline if the path indicated it. The PR is represented in [rad]. Both LC and PR have been proven significant in the success rate of advancement with user-controlled robotic bronchoscopes. In this study, the metrics of LC and PR have been selected as maximum values of the generated LC and PR outputs from the ‘Centerline Module’ at each branching point along the path towards the target location, and the maximum values were recorded for further analysis.

### In-Vivo Animal Model

An animal study was conducted as a part of the study approved by Mass General Brigham (Protocol number: 2021N000190). A 40 kg Yorkshire was sedated and tracheostomy was conducted. A ventilator (MODEL 3000, Midmark Animal Health, Versailles, OH) was connected to the swine model, and then the swine model was scanned at a CT scanner (Discovery MI, GE HealthCare, Chicago, IL). The swine model was placed on a patient bed in the supine position and the robotic catheter was inserted diagonally above the swine model. Vital signs and respiratory parameters were monitored periodically to assess for hemodynamic stability and monitor for respiratory distress. After the setting, the magnitude of breathing motion was confirmed using electromagnetic (EM) tracking sensors (AURORA, NDI, Ontario, Canada) embedded into the peripheral area of four different lobes of the swine model. FIG. 18C shows six consecutive breathing cycles measured by EM tracking sensors as example(s) of breathing motion.

### Validation Studies

### Phantom and Ex Vivo Animal Study

### Bronchoscopic Procedure, Data Collection and Analysis

Each trial of the semi-autonomous robotic advancement started with placing the robotic bronchoscope in the trachea in front of the carina. Then the operator, an engineer highly familiar with the software, started the program and the robot commenced the movement. From that point, the only action taken by the operator was to move (drag and drop) the green cross (see e.g., the cross 1003 in FIGS. 10C-10D) in the bronchoscopic image (see e.g., FIGS. 10C-10D) in the desired direction. The local camera coordinate frame was calibrated with the robot's coordinate system, and the robotic software was designed to advance toward the detected airway closest to the green cross placed by the operator. One advancement per target was performed and recorded. If the driving algorithm failed, the recording was stopped at the point of failure.

The primary metric in the study was target reachability, defining the success in reaching the target location in each advancement. The secondary metric was success at each branching point determined as a binary measurement based on visual assessment of the robot entering the user-defined airway. Other metrics included target generation, target lobe, local curvature (LC) and plane rotation (PR) at each branching point, type of branching point, the total time and total path length to reach the target location (if successfully reached), and time to failure location with airway generation of failure (if not reached). Path length was determined as the linear distance advanced by robot from starting point to target or failure location.

The primary analysis performed in this study was the Chi-square test to analyze the significance of the maximum generation reached and target lobe on target reachability. Second, the influence of branching point type, LC and PR, and lobe segment on the success at branching points was investigated using the Chi-square test. Third, the Chi-square test was performed to analyze the difference in target reachability and success at branching points among the ex-vivo advancements with and without breathing motion simulation.

The inventors also hypothesized that the low local curvatures and plane rotations along the path increase the likelihood of success at branching points. It was also suspected that the breathing motion simulation is not going to decrease the success at branching points and, hence, total target reachability. In all tests, p-values of 0.05 or less were considered to be statistically significant. Pearson's correlation coefficient was calculated for the linear regression analyses. All statistical analyses were performed using Python version 3.7.

### In Vivo Animal Studies

1) Procedure: Two human operators with medical degrees (graduate of medical school and postgraduate year-3 in department of thoracic surgery) were tasked to navigate the robotic catheter using the gamepad (Logitech Gamepad F310, Logitech, Lausanne, Switzerland) toward pseudo-tumors injected in each lobe before the study and ended the navigation when the robotic catheter reached within 20 mm from the pseudo-tumors. The human operators were allowed to move the robotic catheter forward, to retract, and to bend the robotic catheter toward any direction using the gamepad controller mapped with an endoscopic camera view. The robotic catheter was automatically bent during retraction using the reverse FTL motion algorithm.

During navigation planning and/or autonomous navigation, a navigator sending voice commands to the autonomous navigation and/or plan randomly selected the airway at each bifurcation point for the robotic catheter to move in and ended the autonomous navigation and/or navigation plan when the mucus blocked the endoscopic camera view. The navigator was not allowed to change the selected airway before the robotic catheter moved into the selected airway, and not allowed to retract the robotic catheter in the middle of one attempt.

The navigation from the trachea to the point where the navigation was ended was defined as one attempt. The starting point of all attempts was set at 10 mm away from the carina in the trachea. To create the clinical scenario as accurate as possible during the study, the two human operators and the navigator sending voice commands were unaware that their input commands and force applied to each driving wire were recorded, and that the recorded data would be compared with each other after the study.

2) Data collection: Time and force defined below were collected as metrics to compare the autonomous navigation with the navigation by the human operators. All data points during retraction were excluded. When the robotic catheter was moved forward and bent at a bifurcation point, one data point was collected as an independent data point.

a) Time for bending command: Input commands to control the robotic catheter including moving forward, retraction and bending were recorded at 100 Hz. The time for bending command was collected as the summation of the time for the operator or autonomous navigation software to send input commands to bend the robotic catheter at a bifurcation point.

b) Maximum force applied to driving wire: Force applied to each driving wire to bend the tip section of the robotic catheter was recorded at 100 Hz using a strain gauge (KFRB General-purpose Foil Strain Gage, Kyowa Electronic Instruments, Tokyo, Japan) attached to each driving wire. Then the absolute value of the maximum force of three driving wires at each bifurcation point was extracted to indirectly evaluate the interaction against the airway wall.

3) Data analysis: First, box plots were generated for the time for bending command and the maximum force at each bifurcation point for human operators and autonomous navigation software. The medians with interquartile range (IQR) of the box plots were reported. Then the data points were divided into two locations of the lung, the central defined as the airway between the carina to the third generation and the peripheral area defined as the airway more than fourth generation. The medians with IQR at the central and the peripheral area for each operator type were reported. Mann-Whitney U test was performed to compare the difference between each operator type. P-values of 0.05 or less were considered to be statistically significant.

Second, scatter plots were generated for the both metrics with the airway generation, with regression lines and 95% confidential intervals. The inventors analyzed the data using multiple regression models with time and force as response, generation number and operator type (human or autonomous), and their interaction as predictors. The inventors treated generation as a continuous variable, so that the main effect of operator type is the difference in intercepts between lines fit for each type, and the interaction term is the corresponding difference in slopes. The inventors tested the null hypothesis that both of these differences are simultaneously equal to zero using an F-test. The results show that the autonomous navigation and/or planning keeps the catheter close to the center of the airway, which leads to a safer bronchoscopy and reduces/minimizes contact with an airway wall.

### Results

The summary statistics are presented in Table 2 below:

TABLE 2
Results and Summary Statistics*

| | | Success | | | Failure | | |
|---|---|---|---|---|---|---|---|
| | Target | | Distance to | | | Distance to | Time to |
| | Reachability | Generation | Target | Time | Generation | failure | failure |
| Model | [%] | of Success | [mm] | [s] | of Failure | [mm] | [s] |
| Phantom | 73.3 | 7.0 ± 1.9 | 195.2 ± 31.4 | 132.3 ± 21.0 | 5.0 ± 1.7 | 146.0 ± 24.4 | 95.2 ± 19.4 |
| Ex-vivo 1 | 77.7 | 7.5 ± 2.9 | 103.6 ± 18.9 | 88.8 ± 19.3 | 7.3 ± 3.4 | 88.4 ± 23.0 | 72.1 ± 24.3 |
| Ex-vivo 2 | 78.8 | 7.3 ± 2.5 | 93.3 ± 24.3 | 69.6 ± 16.9 | 6.7 ± 3.5 | 65.8 ± 40.5 | 44.9 ± 25.8 |

### Phantom Study

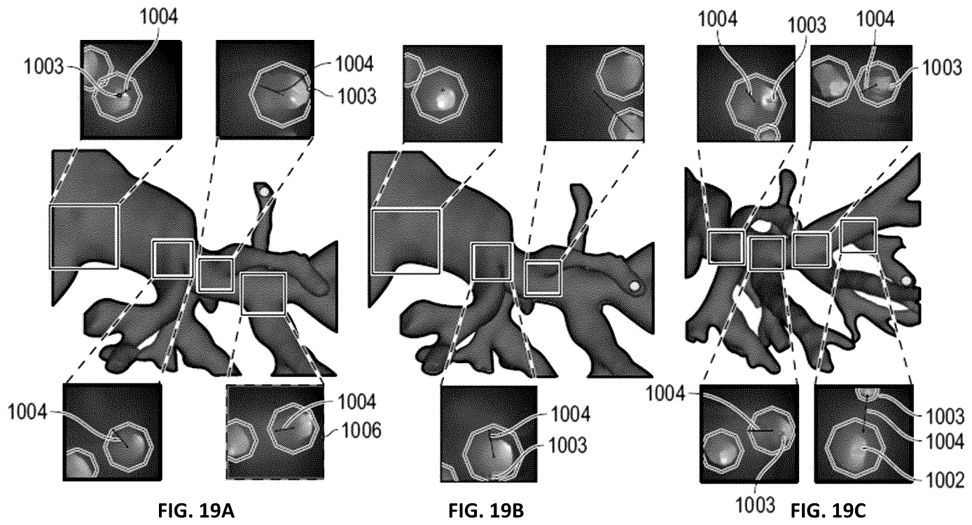

The target reachability achieved in phantom was 73.3%. 481 branching points were tried in the phantom for autonomous robotic advancements. The overall success rate at branching points achieved was 95.8%. The branching points comprised 399 bifurcations and 82 trifurcations. The success rates at bifurcations and trifurcations were 97% and 92%, respectively. Statistical analysis using the Chi-square test revealed a significant difference (p=0.03) between the two types of branching points in phantom (see FIGS. 19A-19C).

Furthermore, the success at branching points varied across different lobe segments, with rates of 99% for the left lower lobe, 93% for the left upper lobe, 97% for the right lower lobe, 85% for the right middle lobe, and 94% for the right upper lobe. The Chi-square test demonstrated a statistically significant difference (p=0.005) in success at branching points between the lobe segments.

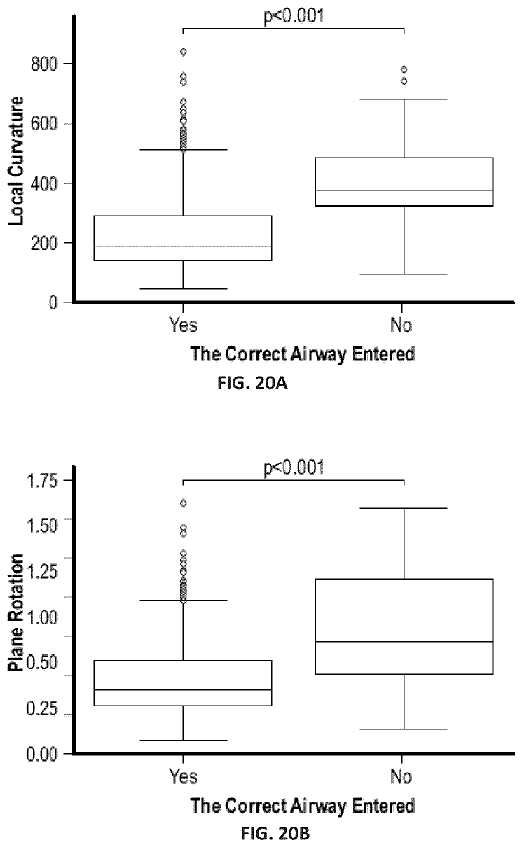

The average LC and PR at successful branching points were respectively 287.5 f 125.5 [mm⁻¹] and 0.4±0.2 [rad]. The average LC and PR at failed branching points were respectively 429.5±133.7 [mm⁻¹] and 0.9±0.3 [rad]. The paired Wilcoxon signed-rank test showed no statistical significance of LC (p<0.001) and PR (p<0.001). Boxplots showing the significance of LC and PR on success at branching points are presented in FIGS. 20A-20B together with ex-vivo data.

Using autonomous method features of the present disclosure, the inventors, via the experiment, successfully accessed the targets (as shown in FIG. 18B). These results underscore the promising potential of our method(s) and related features of the present disclosure that may be used to redefine the standards of robotic bronchoscopy.

FIGS. 19A-19C illustrate views of at least one embodiment of a navigation algorithm performing at various branching points in a phantom where FIG. 19A shows a path on which the target location (dot) was not reached (e.g., the algorithm may not have traversed the last bifurcation where an airway on the right was not detected), where FIG. 19B shows a path on which the target location (dot) was successfully reached, and where FIG. 19C shows a path on which the target location was also successful reached. The highlighted squares represent estimated depth maps with detected airways at each visible branching point on paths toward target locations. The black frame (or a frame of another set/first color) represents success at a branching point and the frame of a set or predetermined color (e.g., red or other different/second color) (e.g., frame 1006 may be the frame of a red or different/second color as shown in the bottom right frame of FIG. 19A) represents a failure at a branching point. All three targets were in RLL. Red pixel(s) (e.g., the pixel 1002) represent the center of a detected airway, green cross (e.g., the cross or plus sign 1003) represents the desired direction determined by the user (drag and drop), and the blue segment (e.g., the segment 1004) is the direction vector between the center of the image/depth map and the center of the detected blob in closer or closest proximity to the green cross (e.g., the cross or plus sign 1003).

FIGS. 20A-20B illustrate graphs showing success at branching point(s) with respect to Local Curvature (LC) and Plane Rotation (PR), respectively, for all data combined in one or more embodiments. FIGS. 20A-20B show the statistically significant difference between successful performance at branching points with respect to LC (see FIG. 20A) and PR (see FIG. 20B). LC is expressed in [mm⁻¹] and PR in [rad].

### Ex-Vivo Specimen/Animal Study

The target reachability achieved in ex-vivo #1 was 77% and in ex-vivo #2 78% without breathing motion. The target reachability achieved in ex-vivo #1 was 69% and in ex-vivo #2 76% with breathing motion.

774 branching points were tried in the ex-vivo #1 and 583 in ex-vivo #2 for autonomous robotic advancements. The overall success rate at branching points achieved was 97% in ex-vivo #1 and 97% in ex-vivo #2 without BM, and 96% in ex-vivo #1 and 97% in ex-vivo #2 with BM. The branching points comprised 327 bifurcations and 62 trifurcations in ex-vivo #1 and 255 bifurcations and 38 trifurcations in ex-vivo #2 without BM. The branching points comprised 326 bifurcations and 59 trifurcations in ex-vivo #1 and 252 bifurcations and 38 trifurcations in ex-vivo #2 with BM. The success rates without BM at bifurcations and trifurcations were respectively 98% and 92% in ex-vivo #1, and 97% and 95% in ex-vivo #2. The success rates with BM at bifurcations and trifurcations were respectively 96% and 93% in ex-vivo #1, and 96% and 97% in ex-vivo #2. Statistical analysis using the Chi-square test revealed a significant difference between the two types of branching points for both ex-vivo specimens (p=0.03).

Furthermore, the success at branching points varied across different lobe segments, with rates (ex-vivo #i, ex-vivo #2) of (97%,96%) for the LLL, (100%,77%) for the LUL, (99%,100%) for the RLL, (95%,100%) for the RML, and (94%,100%) for the RUL, without BM. With BM the results were as follows (ex-vivo #i with BM, ex-vivo #2 with BM) of (96%,97%) for the LLL, (100%,50%) for the LUL, (96%,99%) for the RLL, (92%,100%) for the RML, and (97%,100%) for the RUL. The Chi-square test demonstrated a statistically significant difference (p<0.001) in success at branching points between the lobe segments for all ex-vivo data combined.

The average LC and PR at successful branching points were respectively 211.9±112.6 [mm⁻¹] and 0.4±0.2 [rad] for ex-vivo #1, and 184.5±110.4 [mm⁻¹] and 0.6±0.2 [rad] for ex-vivo #2. The average LC and PR at failed branching points were respectively 393.7±153.5 [mm⁻¹] and 0.6±0.3 [rad] for ex-vivo #1, and 369.5±200.6 [mm⁻¹] and 0.7±0.4 [rad] for ex-vivo #2. The paired Wilcoxon signed-rank test showed statistical significance of LC (p<0.001) and PR (p<0.001) for both ex-vivos on success at branching points. FIGS. 20A-20B represent the comparison of LC and PR for successful and failed branching points, for all data (phantom, ex-vivos, ex-vivos with breathing motion) combined.

During the study, results of Local Curvature (LC) and Plane Rotation (PR) were displayed on three advancement paths towards different target locations with highlighted, color-coded values of LC and PR along the paths. Specifically, the views illustrated impact(s) of Local Curvature (LC) and Plane Rotation (PR) on one or more performances of one or more embodiments of a navigation algorithm where one view illustrated a path toward a target location in RML of ex vivo #1, which was reached successfully, where another view illustrated a path toward a target location in LLL of ex vivo #1, which was reached successfully, and where yet another view illustrated a path toward a target location in RLL of the phantom, which failed at a location marked with a square (e.g., a red square).

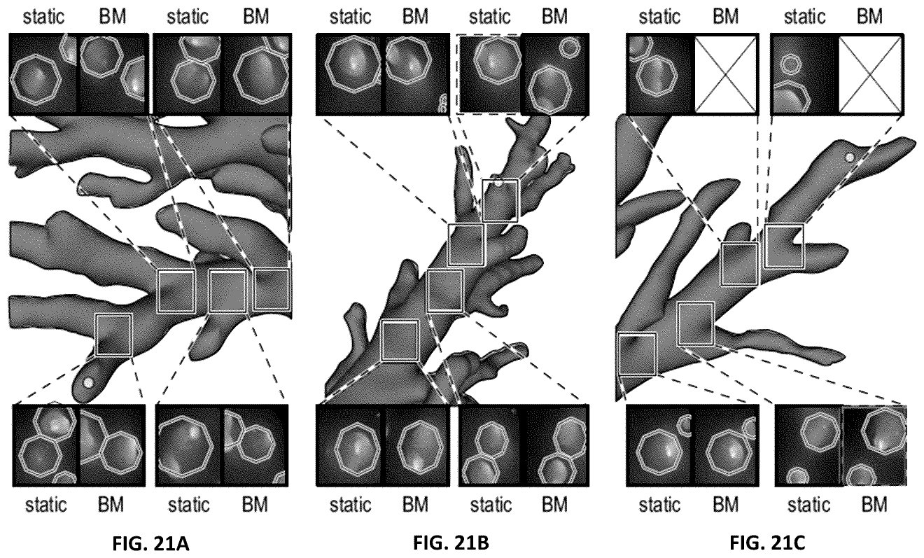

The Chi-square test demonstrated no statistically significant difference (ex-vivo #1, ex-vivo #2) in target reachability (p=0.37, p=0.79) and success at branching points (p=0.43, p=0.8) between the ex-vivo advancements with and without breathing simulations (see e.g., FIGS. 15A-15C). FIGS. 21A-21C illustrate three advancement paths towards different target locations (see blue dots) using one or more embodiments of navigation feature(s) with and without BM. FIGS. 21A-21C illustrate one or more impacts of breathing motion on a performance of the one or more navigation algorithm(s), where FIG. 21A shows a path on which the target location (ex vivo #1 LLL) was reached with and without breathing motion (BM), where FIG. 21B shows a path on which the target location (ex vivo #1 RLL) was not reached without BM but was reached with BM (such as result illustrates that at times BM may help the algorithm(s) with detecting and entering the right airway for one or more embodiments of the present disclosure), and where FIG. 21C shows a path on which the target location (ex vivo #1 RML) was reached without BM was not reached with BM (such a result illustrates that at times BM may affect performance of an algorithm in one or more situations. That said, algorithms of the present disclosure are still highly effective under such a condition). The highlighted squares represent estimated depth maps with detected airways at each visible branching point on paths toward target locations. The black frame represents success at a branching point and the red frame represents a failure at a branching point.

### Statistical Analysis

The hypothesis that the low local curvatures and plane rotations along the path might increase the likelihood of success at branching points was correct. Additionally, the hypothesis that breathing motion simulation will not impose a statistically significant difference in success at branching points and hence total target reachability was also correct.

### In-Vivo Animal Study

In total, 112 and 34 data points were collected from the human operators and autonomous navigation, respectively. Each human operator navigated the robotic catheter toward each pseudo-tumor injected into four different lobes and the autonomous navigation attempted five times, twice toward LLL and RML, and one time toward RLL (Table 3), as follows:

TABLE 3
Total attempts and lobes for each operator type:

Operator Type

| | Human | Autonomous |
|---|---|---|
| Attempt (n) | 8 | 5 |
| Data point (n) | | |
| LUL | 15 | 0 |
| LLL | 18 | 16 |
| RML | 17 | 11 |
| RLL | 62 | 7 |

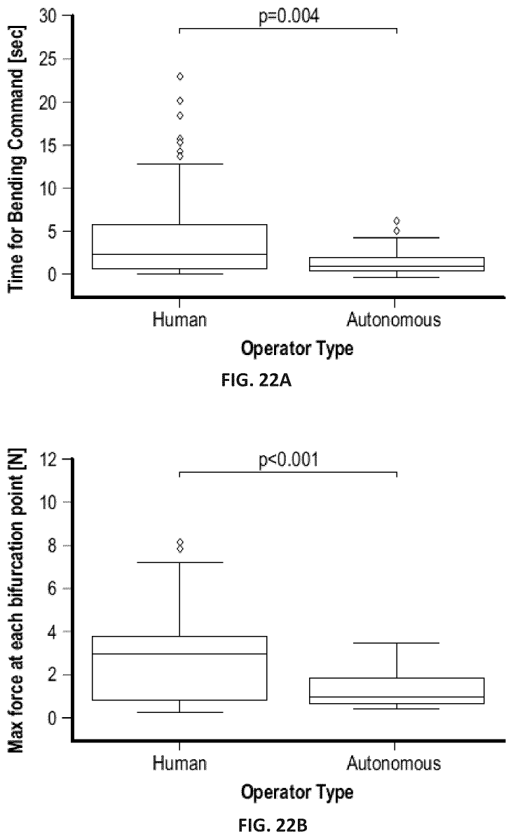

1) Time for bending command and maximum force at each bifurcation point: The median times for bending command were 2.5 [sec](IQR=1.0-5.6) and 1.3 [sec](IQR=0.7-2.3) for human operator and autonomous navigation, respectively. The Mann-Whitney U test showed statistically significant differences between human operators and autonomous navigation (FIG. 22B). FIG. 22A illustrates the box plots for time for the operator or the autonomous navigation to bend the robotic catheter, and FIG. 22B illustrates the box plots for the maximum force for the operator or the autonomous navigation at each bifurcation point.

At the central area of the lung, the median times for bending command were 1.8 [sec](IQR=0.8-3.0) and 1.2 [sec](IQR=0.7-1.7) for human operator and autonomous navigation respectively, showing no statistically significant difference between operator type. At the peripheral area of the lung, the times for bending command were 2.9 [sec](IQR=1.2-7.1) and 1.4 [sec](IQR=0.7-2.8) for human operator and autonomous navigation respectively, showing the statistically significant differences between operator type (p=0.030).

The medians of the maximum force at each bifurcation point were 2.8 (IQR=1.1-3.8) [N] and 1.4 (IQR=0.9-2.1) [N] for the human operators and autonomous navigation, respectively. The Mann-Whitney U test showed statistically significant differences between human operators and autonomous navigation (FIG. 22B).

At the central area of the lung, the medians of the maximum force at each bifurcation point were 1.8 [N](IQR=0.8-3.1) and 1.1 [N](IQR=0.9-1.4) for human operator and autonomous navigation respectively, showing no statistically significant difference between operator type. At the peripheral area of the lung, the medians of the maximum force at each bifurcation point were 3.1 [N](IQR=1.5-4.2) and 1.8 [N](IQR=1.2-2.5) for human operator and autonomous navigation respectively, showing the statistically significant differences between operator type (p=0.005).

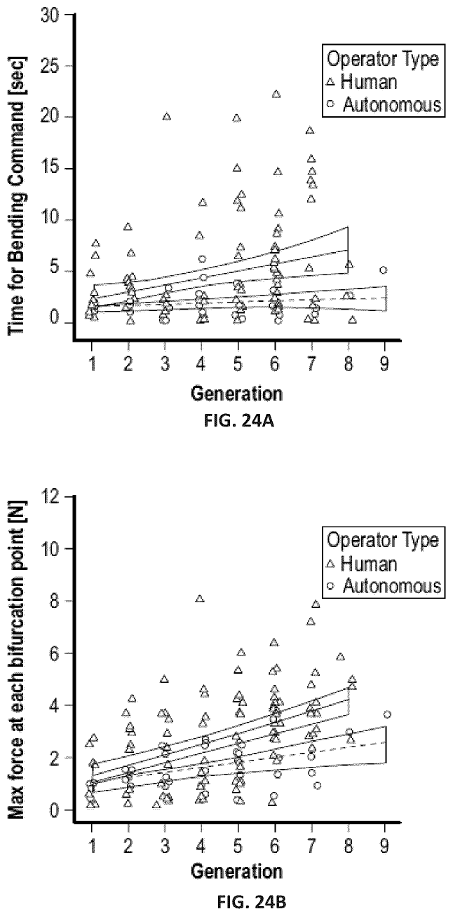

2) Dependency on the airway generation of the lung: The dependency of the time and the force on the airway generation of the lung are shown in FIG. 24A and FIG. 24B with regression lines and 95% confidential intervals. For both metrics, the differences of the regression lines for each operator type become larger as the airway generation increases. FIGS. 24A-24B show scatter plots for time to bend the robotic catheter (FIG. 24A) and maximum force for a human operator and/or the autonomous navigation software (FIG. 24B), respectively. Solid lines showed the linear regression lines with 95% confidential intervals. While not required, jittering was applied on a horizontal axis for visualization.

There are statistically significant differences for both metrics due to operator (p=0.006 for time, p<0.001 for force). The null hypothesis for these tests assume not only that there is no difference in the generation slope, but also that there is no difference in the intercepts of the lines fit for the two operator types.

### DISCUSSION

The inventors have implemented the autonomous advancement of the bronchoscopic robot into a practical clinical tool, providing physicians with the capability to manually outline the robot's desired path. This is achieved by simply placing a marker on the screen in the intended direction using the computer mouse (or other input device). While motion planning remains under physician control, both airway detection and motion execution are fully autonomous features. This amalgamation of manual control and autonomy is groundbreaking; according to the inventors' knowledge, the methods of the present disclosure represent the pioneering clinical instrument facilitating airway tracking for supervised-autonomous driving within a target (e.g., the lung). To validate its effectiveness, the inventors assessed the performance of the driving algorithm(s), emphasizing target reachability and success at branching points. The rigorous testing encompassed a clinically derived phantom (in-vitro), two pig lung specimens (ex-vivo), and one live animal (in-vivo), cumulatively presenting 168 targets. This comprehensive approach, and features discussed herein, serve as the inventors' response(s) to noticed gaps in prior studies.

With the achieved performance, the presented supervised-autonomous driving in the lung is proven to be clinically feasible. The inventors achieved 73.3% target reachability phantom, and 77% in ex-vivo #1 and 78% in ex-vivo #2 without breathing motion, and 69% and 76% with breathing motion. The overall success rate at branching points achieved in phantom was 95.8%, 97% in ex-vivo #1 and 97% in ex-vivo #2 without breathing motion, and 96% and 97% with breathing motion. The inventors inferred that the perpetuity of the anatomical airway structure quantified by LC and PR statistically significantly influences the success at branching points and hence target reachability. The presented method features show that, by using autonomous driving, physicians may safely navigate to the target by controlling a cursor on the screen.

To evaluate the performance of the autonomous driving, the autonomous driving was compared with two human operators using a gamepad controller in a living swine model under breathing motion. Our blinded comparison study revealed that the autonomous driving took less time to bend the robotic catheter and applied less force to the anatomy than the navigation by human operator using a gamepad controller, suggesting the autonomous driving successfully identified the center of the airway in the camera view even with breathing motion and accurately moved the robotic catheter into the identified airway.

One or more embodiments of the present disclosure is in accordance with two studies that recently introduced the approach for autonomous driving in the lung (see e.g., J. Sganga, et al., RAL, pp. 1-10 (2019), which is incorporated by reference herein in its entirety, and Y. Zou, et al., IEEE Transactions on Medical Robotics and Bionics, vol. 4, no. 3, pp. 588-598 (2022), which is incorporated by reference herein in its entirety). The first study reports 95% target reachability with the robot reaching the target in 19 out of 20 trials, but it is limited to 4 targets (J. Sganga, et al., RAL, pp. 1-10 (2019), which is incorporated by reference herein in its entirety). The only other performance metric the subject study presents is time necessary to reach the target, which is redundant not knowing the exact topological location of the target. The clinical origin of the validation lung phantom was not provided in that study. However, a robotic bronchoscope was used in the experiments of that study. The second study proposed a method for detecting the lumen center and maneuvering a manual bronchoscope by integrating it with a robotic device (Y. Zou, et al., IEEE Transactions on Medical Robotics and Bionics, vol. 4, no. 3, pp. 588-598 (2022), which is incorporated by reference herein in its entirety). The subject study does not report any details on the number of targets, the location of the targets within lung anatomy, the origin of the human lung phantom, and the statistical analysis to identify the reasons for failure. The only metric used is the time to target. Both of these Sganga, et al. and Zou, et al. studies differ from the present disclosure in numerous ways, including, but not limited to, in the design of the method(s) of the present disclosure and the comprehensiveness of clinical validation. The methods of those two studies are based on airway detection from supervised learning algorithms. In contrast, one or more methods of the present disclosure first estimate the bronchoscopic depth map using an unsupervised generative learning technique (see e.g., Banach 2021 or Kalia 2024, the disclosures of which are incorporated by reference herein in their entireties) and then perform standard image processing to detect the airways. Moreover, the clinical validation of the two studies (see e.g., J. Sganga, et al., RAL, pp. 1-10 (2019), which is incorporated by reference herein in its entirety, and Y. Zou, et al., IEEE Transactions on Medical Robotics and Bionics, vol. 4, no. 3, pp. 588-598 (2022), which is incorporated by reference herein in its entirety) is limited in vast contrast with, and when compared to, 261 advancements, breathing simulation and statistical analysis performed in experiments of the present disclosure.

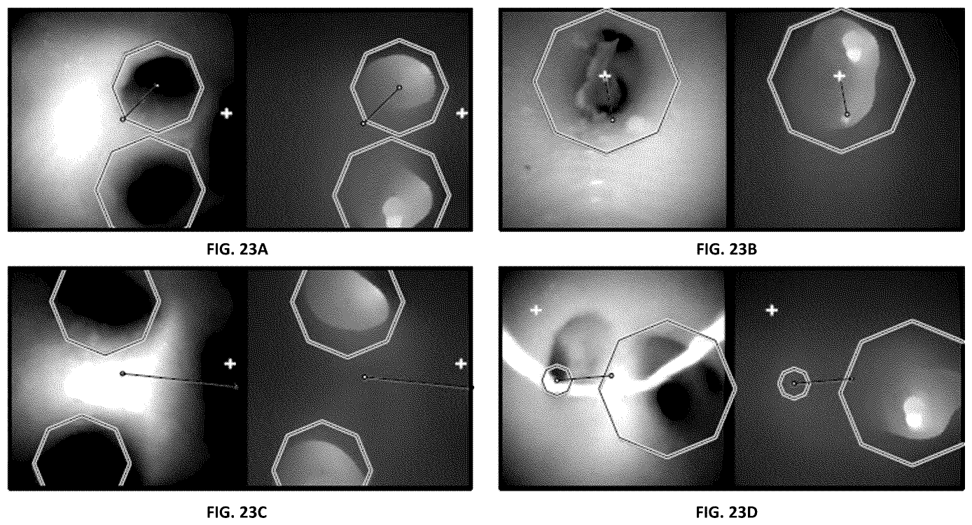

One or more embodiments of the presented method of the present disclosure may be dependent on the quality of bronchoscopic depth estimation by 3cGAN (see e.g., Banach 2021), pose estimation by vision based tracking (see e.g., Kalia 2024), or other AI-related network architecture used or that may be used (for example, while not limited hereto: in one or more embodiments, in a case where one or more processors train one or more models or AI-networks, the one or more trained models or AI-networks is or uses one or a combination of the following: a neural net model or neural network model, a deep convolutional neural network model, a recurrent neural network model with long short-term memory that can take temporal relationships across images or frames into account, a generative adversarial network (GAN) model, a consistent generative adversarial network (cGAN) model, a three cycle-consistent generative adversarial network (3cGAN) model, a model that can take temporal relationships across images or frames into account, a model that can take temporal relationships into account including tissue location(s) during pullback in a vessel and/or including tissue characterization data during pullback in a vessel, a model that can use prior knowledge about a procedure and incorporate the prior knowledge into the machine learning algorithm or a loss function, a model using feature pyramid(s) that can take different image resolutions into account, and/or a model using residual learning technique(s); a segmentation model, a segmentation model with post-processing, a model with pre-processing, a model with post-processing, a segmentation model with pre-processing, a deep learning or machine learning model, a semantic segmentation model or classification model, an object detection or regression model, an object detection or regression model with pre-processing or post-processing, a combination of a semantic segmentation model and an object detection or regression model, a model using repeated segmentation model technique(s), a model using feature pyramid(s), a genetic algorithm that operates to breed multiple models for improved performance, a model using repeated object detection or regression model technique(s); one or more other AI-networks or models known to those skilled in the art; etc.). One of the reasons for lack of success at branching points and hence missing the target may be that occasionally the depth estimation missed the airway when the airway was only partially visible in the bronchoscopic image. An example of such a scenario is presented in FIG. 23A. FIGS. 23A-23D illustrate one or more examples of depth estimation failure and artifact robustness that may be observed in one or more embodiments.

FIG. 23A shows a scenario where the depth map (right side of FIG. 23A) was not estimated accurately and therefore the airway detection algorithm did not detect the airway partially visible on the right side of the bronchoscopic image (left side of FIG. 23A). FIG. 23B shows a scenario where the depth map estimated the airways accurately despite presence of debris. FIG. 23C shows a scenario opposite to the one presented in FIG. 23A where the airway on the right side of the bronchoscopic image (left side of FIG. 23C) is more visible and the airway detection algorithm detects it successfully. FIG. 23D shows a scenario where a visual artifact is ignored by the depth estimation algorithm and both visible airways are detected in the depth map.

Another possible scenario may be related to the fact that the control algorithm should guide the robot along the centerline. Dynamic LSE operates to solve that issue and to guide the robot towards the centerline when not at a branching point. The inventors also identified the failure at branching points as a result of lacking short-term memory, and that using short-term memory may increase success rate(s) at branching points. At branching point with high LC and PR, the algorithm may detect some of the visible airways only for a short moment, not leaving enough time for the control algorithm to react. In such scenarios, a potential solution would involve such short-term memory that ‘remembers’ the detected airways and forces the control algorithm to make the bronchoscopic camera ‘look around’ and make sure that no airways were missed. Such a ‘look around’ mode implemented between certain time or distance intervals may also prevent from missing airways that were not visible in the bronchoscopic image in one or more embodiments of the present disclosure.

In this work, the inventors developed and clinically validated the autonomous driving approach features in bronchoscopy. This is the first clinical tool providing airway tracking for autonomous driving in the lung and extensively validated on phantom and ex-vivo/in-vivo porcine specimens. With the achieved performance, the presented method features for autonomous driving in the lung is/are proven to be clinically feasible and show the potential to revolutionize the standard of care for lung cancer patients.

One or more of the aforementioned features may be used with a continuum robot and related features as disclosed in U.S. Pat. No. 11,882,365, issued on Jan. 23, 2024, the disclosure of which is incorporated by reference herein in its entirety. For example, at least one embodiment of a continuum robot apparatus 10 configuration may be used to implement automatic correction of a direction to which a tool channel or a camera moves or is bent in a case where a displayed image is rotated. The continuum robot apparatus 10 enables to keep a correspondence between a direction on a monitor (top, bottom, right or left of the monitor) and a direction the tool channel or the camera moves on the monitor according to a particular directional command (up, down, turn right or turn left) even if the displayed image is rotated. The continuum robot apparatus 10 also may be used with any of the navigation planning, autonomous navigation, movement detection, and/or control features of the present disclosure.

The user may provide an operation input through an input element, and the continuum robot apparatus 10 may receive information of the input element and one or more input/output devices, which may include, but are not limited to, a receiver, a transmitter, a speaker, a display, an imaging sensor, a user input device, which may include a keyboard, a keypad, a mouse, a position tracked stylus, a position tracked probe, a foot switch, a microphone, a camera, etc. A user may adjust various parameters of the robot 10, such as the speed (e.g., rotational speed, translational speed, etc.), angle or plane, or other parameters.

The continuum robot apparatus 10 may be interconnected with medical instruments or other devices, and may be controlled independently, externally, or remotely by a controller 50. In one or more embodiments of the present disclosure, one or more features of the continuum robot apparatus 10 and one or more features of the continuum robot or catheter or probe system 1000 may be used in combination or alternatively to each other.

The memory 52 may be used as a work memory or may include any memory discussed in the present disclosure. The storage 53 stores software or computer instructions, and may be any type of storage, data storage 150, or other memory or storage discussed in the present disclosure. The CPU 51, which may include one or more processors, circuitry, or a combination thereof, executes the software developed in the memory 52 (or any other memory discussed herein). The I/O interface 54 operates to input information from the continuum robot apparatus 10 to the controller 50 and to output information for displaying to the display 60 (or any other display discussed herein, such as, but not limited to, display 1209 discussed below).

The communication interface 55 may be configured as a circuit or other device for communicating with components included in the apparatus 10, and with various external apparatuses connected to the apparatus via a network. For example, the communication interface 55 may store information to be output in a transfer packet and may output the transfer packet to an external apparatus via the network by communication technology such as Transmission Control Protocol/Internet Protocol (TCP/IP). The apparatus may include a plurality of communication circuits according to a desired communication form.

The controller 50 may be communicatively interconnected or interfaced with external device(s) including, for example, one or more data storages (e.g., the data storage 150, the SSD or storage drive 1207 discussed below, or any other storage discussed herein), one or more external user input/output devices, or the like. The controller 50 may interface with other elements including, for example, one or more of an external storage, a display, a keyboard, a mouse, a sensor, a microphone, a speaker, a projector, a scanner, a display, an illumination device, etc.

The display 60 may be a display device configured, for example, as a monitor, an LCD (liquid panel display), an LED display, an OLED (organic LED) display, a plasma display, an organic electro luminescence panel, or any other display discussed herein (including, but not limited to, displays 101-1, 101-2, etc.). Based on the control of the apparatus, a screen may be displayed on the display 60 showing image(s), such as, but not limited to, one or more images being captured, captured images, captured moving images recorded on the storage unit, etc.

The components may be connected together by a bus 56 so that the components may communicate with each other. The bus 56 transmits and receives data between these pieces of hardware connected together, or the bus 56 transmits a command from the CPU 51 to the other pieces of hardware. The components may be implemented by one or more physical devices that may be coupled to the CPU 51 through a communication channel. For example, the controller 50 may be implemented using circuitry in the form of ASIC (application specific integrated circuits) or other similar circuits as discussed herein. Alternatively, the controller 50 may be implemented as a combination of hardware and software, where the software is loaded into a processor from a memory or over a network connection. Functionality of the controller 50 may be stored on a storage medium, which may include, but is not limited to, RAM (random-access memory), magnetic or optical drive, diskette, cloud storage, etc.

The units described herein are exemplary and/or preferable modules for implementing processes described in the present disclosure. However, one or mor embodiments of the present disclosure are not limited thereto. The term “unit”, as used herein, may generally refer to firmware, software, hardware, or other component, such as circuitry or the like, or any combination thereof, that is used to effectuate a purpose. The modules may be hardware units (such as circuitry, firmware, a field programmable gate array, a digital signal processor, an application specific integrated circuit or the like) and/or software modules (such as a computer readable program, instructions stored in a memory or storage medium, etc.). The modules for implementing the various steps are not described exhaustively above. However, where there is a step of performing a certain process, there may be a corresponding functional module or unit (implemented by hardware and/or software) for implementing the same process. Technical solutions by all combinations of steps described and units corresponding to these steps are included in the present disclosure.

One or more navigation planning, autonomous navigation, movement detection, and/or control features of the present disclosure may be used with one or more image correction or adjustment features in one or more embodiments. One or more adjustments, corrections, or smoothing functions for a catheter or probe device and/or a continuum robot may adjust a path of one or more sections or portions of the catheter or probe device and/or the continuum robot (e.g., the continuum robot 104, the continuum robot device 10, etc.), and one or more embodiments may make a corresponding adjustment or correction to an image view. For example, in one or more embodiments the medical tool may be a bronchoscope.

While one or more features of the present disclosure have been described with reference to exemplary embodiments, it is to be understood that the present disclosure is not limited to the disclosed exemplary embodiments. The scope of the claims is to be accorded the broadest interpretation so as to encompass all modifications, equivalent structures, and functions.

A computer, such as the console or computer 1200, 1200′, may perform any of the steps, processes, and/or techniques discussed herein for any apparatus, bronchoscope, robot, and/or system being manufactured or used, any of the embodiments shown in FIGS. 1-31, any other bronchoscope, robot, apparatus, or system discussed herein or included herewith, etc.

There are many ways to control a bronchoscope or robotic bronchoscope, perform imaging, navigation planning, autonomous navigation, movement detection, and/or control for a continuum robot, correct or adjust an image or a path/state (or one or more sections or portions of) a continuum robot (or other probe or catheter device or system), or perform any other measurement or process discussed herein, to perform continuum robot and/or bronchoscope method(s) or algorithm(s), and/or to control at least one bronchoscope and/or continuum robot device/apparatus/robot, system and/or storage medium, digital as well as analog. In at least one embodiment, a computer, such as the console or computer 1200, 1200′, may be dedicated to control and/or use continuum robot and/or bronchoscope devices, systems, methods, and/or storage mediums for use therewith described herein.

The one or more detectors, sensors, cameras, or other components of the bronchoscope, robotic bronchoscope, robot, continuum robot, apparatus, system, method, or storage medium embodiments (e.g. of the system 1000 of FIG. 1 or any other system discussed herein; of any bronchoscope, robot, apparatus, system, method, or storage medium discussed herein; etc.) may transmit the digital or analog signals to a processor or a computer such as, but not limited to, an image processor or display controller 100, a controller 102, a CPU 120, a controller 50, a CPU 51, a processor or computer 1200, 1200′ (see e.g., at least FIGS. 1-5 and 25-31), a combination thereof, any other processor(s) discussed herein, etc. The image processor may be a dedicated image processor or a general purpose processor that is configured to process images. In at least one embodiment, the computer 1200, 1200′ may be used in place of, or in addition to, the image processor or display controller 100 and/or the controller 102 (or any other processor or controller discussed herein, such as, but not limited to, the controller 50, the CPU 51, the CPU 120, etc.). In an alternative embodiment, the image processor may include an ADC and receive analog signals from the one or more detectors or sensors of the bronchoscopes, robots, apparatuses, systems (e.g., system 1000 (or any other system discussed herein)), methods, storage mediums, etc. The image processor may include one or more of a CPU, DSP, FPGA, ASIC, or some other processing circuitry. The image processor may include memory for storing image, data, and instructions. The image processor may generate one or more images based on the information provided by the one or more detectors, sensors, or cameras. A computer or processor discussed herein, such as, but not limited to, a processor of the devices, apparatuses, bronchoscopes, or systems of FIGS. 1-5 and 25-31, the computer 1200, the computer 1200′, the image processor, etc. may also include one or more components further discussed herein below (see e.g., FIGS. 25-31).

Electrical analog signals obtained from the output of the system 1000 or the components thereof, and/or from the devices, bronchoscopes, apparatuses, or systems of FIGS. 1-5 and 25-31, may be converted to digital signals to be analyzed with a computer, such as, but not limited to, the computers or controllers 100, 102 of FIG. 1, the computer 1200, 1200′, etc.

There are many ways to perform imaging, control a bronchoscope or robotic bronchoscope, perform navigation planning, autonomous navigation, movement detection, and/or control for a continuum robot, perform bronchoscope or robotic bronchoscope method(s) or algorithm(s), control at least bronchoscope or robotic device/apparatus/bronchoscope, system, and/or storage medium, correct or adjust an image, correct, adjust, or smooth a path/state (or section or portion) of a continuum robot, or perform any other measurement or process discussed herein, to perform continuum robot method(s) or algorithm(s), and/or to control at least one continuum robot device/apparatus, system and/or storage medium, digital as well as analog. By way of a further example, in at least one embodiment, a computer, such as the computer or controllers 100, 102 of FIG. 1, the console or computer 1200, 1200′, etc., may be dedicated to the autonomous navigation/planning/control and the monitoring of the bronchoscopes, robotic bronchoscopes, devices, systems, methods, and/or storage mediums and/or of continuum robot devices, systems, methods and/or storage mediums described herein.

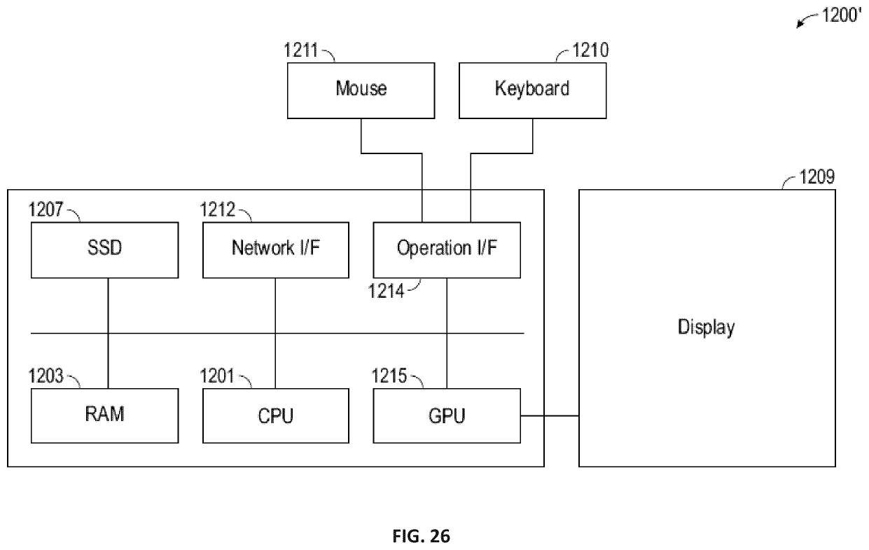

The electric signals used for imaging may be sent to one or more processors, such as, but not limited to, the processors or controllers 100, 102 of FIGS. 1-5, a computer 1200 (see e.g., FIG. 25), a computer 1200′ (see e.g., FIG. 26), etc. as discussed further below, via cable(s) or wire(s), such as, but not limited to, the cable(s) or wire(s) 113 (see FIG. 25). Additionally or alternatively, the computers or processors discussed herein are interchangeable, and may operate to perform any of the feature(s) and method(s) discussed herein.

Various components of a computer system 1200 (see e.g., the console or computer 1200 as may be used as one embodiment example of the computer, processor, or controllers 100, 102 shown in FIG. 1) are provided in FIG. 25. A computer system 1200 may include a central processing unit (“CPU”) 1201, a ROM 1202, a RAM 1203, a communication interface 1205, a hard disk (and/or other storage device) 1204, a screen (or monitor interface) 1209, a keyboard (or input interface; may also include a mouse or other input device in addition to the keyboard) 1210 and a BUS (or “Bus”) or other connection lines (e.g., connection line 1213) between one or more of the aforementioned components (e.g., as shown in FIG. 25). In addition, the computer system 1200 may comprise one or more of the aforementioned components. For example, a computer system 1200 may include a CPU 1201, a RAM 1203, an input/output (I/O) interface (such as the communication interface 1205) and a bus (which may include one or more lines 1213 as a communication system between components of the computer system 1200; in one or more embodiments, the computer system 1200 and at least the CPU 1201 thereof may communicate with the one or more aforementioned components of a robot device, apparatus, bronchoscope or robotic bronchoscope, or system using same, and/or a continuum robot device or system using same, such as, but not limited to, the system 1000, the devices/systems of FIGS. 1-5, and/or other systems/apparatuses discussed herein above, via one or more lines 1213), and one or more other computer systems 1200 may include one or more combinations of the other aforementioned components (e.g., the one or more lines 1213 of the computer 1200 may connect to other components via line 113). The CPU 1201 is configured to read and perform computer-executable instructions stored in a storage medium. The computer-executable instructions may include those for the performance of the methods and/or calculations described herein. The computer system 1200 may include one or more additional processors in addition to CPU 1201, and such processors, including the CPU 1201, may be used for controlling and/or manufacturing a device, system or storage medium for use with same or for use with any continuum robot, bronchoscope, or robotic bronchoscope technique(s), and/or use with imaging, navigation planning, autonomous navigation, movement detection, and/or control technique(s) discussed herein. The system 1200 may further include one or more processors connected via a network connection (e.g., via network 1206). The CPU 1201 and any additional processor being used by the system 1200 may be located in the same telecom network or in different telecom networks (e.g., performing, manufacturing, controlling, calculation, and/or using technique(s) may be controlled remotely).

The I/O or communication interface 1205 provides communication interfaces to input and output devices, which may include the one or more of the aforementioned components of any of the bronchoscopes, robotic bronchoscopes, apparatuses, devices, and/or systems discussed herein (e.g., the controller 100, the controller 102, the displays 101-1, 101-2, the actuator 103, the continuum device 104, the operating portion or controller 105, the camera 106, the EM tracking sensor 106 (when used), the position detector 107, the rail 110, etc.), a microphone, a communication cable and a network (either wired or wireless), a keyboard 1210, a mouse (see e.g., the mouse 1211 as shown in FIG. 26), a touch screen or screen 1209, a light pen and so on. The communication interface of the computer 1200 may connect to other components discussed herein via line 113 (as diagrammatically shown in FIG. 25). The Monitor interface or screen 1209 provides communication interfaces thereto. In one or more embodiments, the camera 106 is used alone or in combination with the EM sensor 106, and the position detector 107 may be optional (e.g., may be used along with the EM sensor 106).

Any methods and/or data of the present disclosure, such as, but not limited to, the methods for using/guiding and/or controlling a bronchoscope, robotic bronchoscope, continuum robot, or catheter device, apparatus, system, or storage medium for use with same and/or method(s) for imaging, performing tissue, lesion, or sample characterization or analysis, performing diagnosis, planning and/or examination, for controlling a bronchoscope or robotic bronchoscope, device/apparatus, or system, for performing navigation planning, autonomous navigation, movement detection, and/or control technique(s), for performing adjustment or smoothing techniques (e.g., to a path of, to a pose or position of, to a state of, or to one or more sections or portions of, a continuum robot, a catheter or a probe), and/or for performing imaging and/or image correction or adjustment technique(s), (or any other technique(s)) as discussed herein, may be stored on a computer-readable storage medium. A computer-readable and/or writable storage medium used commonly, such as, but not limited to, one or more of a hard disk (e.g., the hard disk 1204, a magnetic disk, etc.), a flash memory, a CD, an optical disc (e.g., a compact disc (“CD”), a digital versatile disc (“DVD”), a Blu-ray™ disc, etc.), a magneto-optical disk, a random-access memory (“RAM”) (such as the RAM 1203), a DRAM, a read only memory (“ROM”), a storage of distributed computing systems, a memory card, or the like (e.g., other semiconductor memory, such as, but not limited to, a non-volatile memory card, a solid state drive (SSD) (see SSD 1207 in FIG. 26), SRAM, etc.), an optional combination thereof, a server/database, a neural network (or other AI architecture/structure/models) etc. may be used to cause a processor, such as, the processor or CPU 1201 of the aforementioned computer system 1200 to perform the steps of the methods disclosed herein. The computer-readable storage medium may be a non-transitory computer-readable medium, and/or the computer-readable medium may comprise all computer-readable media, with the sole exception being a transitory, propagating signal in one or more embodiments. The computer-readable storage medium may include media that store information for predetermined, limited, or short period(s) of time and/or only in the presence of power, such as, but not limited to Random Access Memory (RAM), register memory, processor cache(s), etc. Embodiment(s) may also be realized by a computer and/or neural network (or other AI architecture/structure/models) of a system or apparatus that reads out and executes computer executable instructions (e.g., one or more programs) recorded on a storage medium (which may also be referred to more fully as a “non-transitory computer-readable storage medium”) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s).

In accordance with at least one aspect of the present disclosure, the methods, bronchoscopes or robotic bronchoscopes, devices, systems, and computer-readable storage mediums related to the processors, such as, but not limited to, the processor(s) of the aforementioned computer 1200, 1200′, the controller 100, the controller 102, any other processor discussed herein, etc., as described herein may be achieved utilizing suitable hardware, such as that illustrated in the figures. Functionality of one or more aspects of the present disclosure may be achieved utilizing suitable hardware, such as that illustrated in FIG. 25. Such hardware may be implemented utilizing any of the known technologies, such as standard digital circuitry, any of the known processors that are operable to execute software and/or firmware programs, any neural networks (and/or any other artificial intelligence (AI) structure/architecture/models/etc. that may be used to perform any of the technique(s) discussed herein) one or more programmable digital devices or systems, such as programmable read only memories (PROMs), programmable array logic devices (PALs), etc. The CPU 1201 (as shown in FIG. 25 or FIG. 26, and/or which may be included in the computer, processor, controller and/or CPU 120 of FIGS. 1-5 and/or of any of the other figures or embodiments discussed herein), CPU 51, and/or the CPU 120 may also include and/or be made of one or more microprocessors, nanoprocessors, one or more graphics processing units (“GPUs”; also called a visual processing unit (“VPU”)), one or more Field Programmable Gate Arrays (“FPGAs”), or other types of processing components (e.g., application specific integrated circuit(s) (ASIC)). Still further, the various aspects of the present disclosure may be implemented by way of software and/or firmware program(s) that may be stored on suitable storage medium (e.g., computer-readable storage medium, hard drive, etc.) or media (such as floppy disk(s), memory chip(s), etc.) for transportability and/or distribution. The computer may include a network of separate computers or separate processors to read out and execute the computer executable instructions. The computer executable instructions may be provided to the computer, for example, from a network or the storage medium. The computers or processors (e.g., 100, 102, 120, 50, 51, 1200, 1200′, any other computer or processor discussed herein, etc.) may include the aforementioned CPU structure, or may be connected to CPU structure for communication therewith.

As aforementioned, hardware structure of an alternative embodiment of a computer or console 1200′ is shown in FIG. 26. The computer 1200′ includes a central processing unit (CPU) 1201, a graphical processing unit (GPU) 1215, a random access memory (RAM) 1203, a network interface device 1212, an operation interface 1214 such as a universal serial bus (USB) and a memory such as a hard disk drive or a solid-state drive (SSD) 1207. Preferably, the computer or console 1200′ includes a display 1209 (and/or the displays 101-1, 101-2, any other display(s) discussed herein, etc.). The computer 1200′ may connect with one or more components of a system (e.g., the systems/apparatuses/bronchoscopes/robotic bronchoscopes discussed herein; the systems/apparatuses/bronchoscopes/robotic bronchoscopes and/or any other device, apparatus, system, etc. of any of the figures included herewith (e.g., the systems/apparatuses of FIGS. 1-5, 25-31, etc.)) via the operation interface 1214 or the network interface 1212. The operation interface 1214 is connected with an operation unit such as a mouse device 1211, a keyboard 1210 or a touch panel device. The computer 1200′ may include two or more of each component. Alternatively, the CPU 1201 or the GPU 1215 may be replaced by the field-programmable gate array (FPGA), the application-specific integrated circuit (ASIC) or other processing unit depending on the design of a computer, such as the computer 1200, 1200′, etc.

At least one computer program is stored in the SSD 1207 (or any other storage device/drive discussed herein), and the CPU 1201 loads the at least one program onto the RAM 1203, and executes the instructions in the at least one program to perform process(es) described herein, and the basic input, output, calculation, memory writing, and memory reading processes.

The computer, such as the computer 1200, 1200′, the computer, processors, and/or controllers of FIGS. 1-5 and/or of any other figure(s) included herewith, etc., communicates with the one or more components of the apparatuses/systems/bronchoscopes/robotic bronchoscopes/robots of FIGS. 1-5, of any other figure(s) included herewith, etc. and/or of any other apparatuses/systems/bronchoscopes/robotic bronchoscopes/robots/etc. discussed herein, to perform imaging, and reconstructs an image from the acquired intensity data. The monitor or display 1209 displays the reconstructed image, and the monitor or display 1209 may display other information about the imaging condition or about an object, target, or sample to be imaged. The monitor 1209 also provides a graphical user interface for a user to operate a system, for example when performing CT, MRI, or other imaging technique(s), including, but not limited to, controlling continuum robots/bronchoscopes/robotic bronchoscopes/devices/systems, and/or performing imaging, navigation planning, autonomous navigation, movement detection, and/or control technique(s). An operation signal is input from the operation unit (e.g., such as, but not limited to, a mouse device 1211, a keyboard 1210, a touch panel device, etc.) into the operation interface 1214 in the computer 1200′, and corresponding to the operation signal the computer 1200′ instructs the apparatus/bronchoscope/robotic bronchoscope/system (e.g., the system 1000, the systems/apparatuses of FIGS. 1-5, any other system/apparatus discussed herein, any of the apparatus(es)/bronchoscope(s)/robotic bronchoscope(s)/system(s) discussed herein, etc.) to start or end the imaging, and/or to start or end bronchoscope/robotic bronchoscope/device/system/continuum robot control(s) and/or performance of imaging, correction, adjustment, and/or smoothing technique(s). The camera or imaging device may have interfaces to communicate with the computers 1200, 1200′ (or any other computer or processor discussed herein) to send and receive the status information and the control signals.

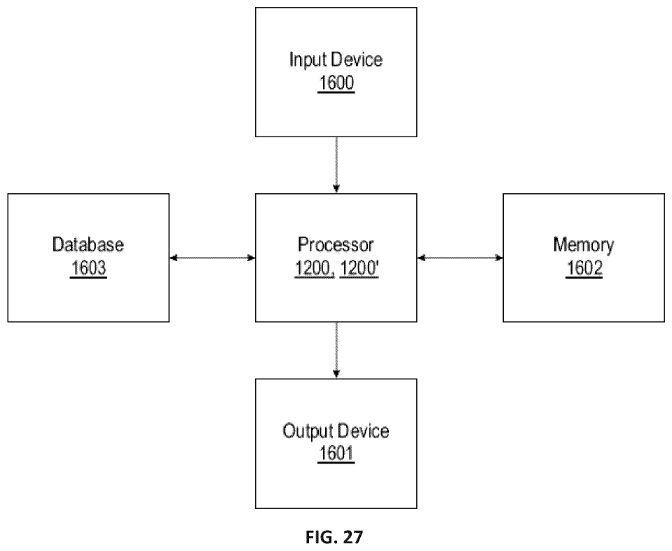

As aforementioned techniques of the present disclosure may be performed using artificial intelligence structure(s), such as, but not limited to, residual networks, neural networks, convolutional neural networks, GANs, cGANs, etc. In one or more embodiments, other types of AI structure(s) and/or network(s) may be used. The below discussed network/structure examples are illustrative only, and any of the features of the present disclosure may be used with any AI structure or network, including AI networks that are less complex than the network structures discussed below (e.g., including such structure as show in FIGS. 27-31).

As shown in FIG. 27, one or more processors or computers 1200, 1200′ (or any other processor discussed herein) may be part of a system in which the one or more processors or computers 1200, 1200′ (or any other processor discussed herein) communicate with other devices (e.g., a database 1603, a memory 1602 (which may be used with or replaced by any other type of memory discussed herein or known to those skilled in the art), an input device 1600, an output device 1601, etc.). In one or more embodiments, one or more models may have been trained previously and stored in one or more locations, such as, but not limited to, the memory 1602, the database 1603, etc. In one or more embodiments, it is possible that one or more models and/or data discussed herein (e.g., training data, testing data, validation data, imaging data, etc.) may be input or loaded via a device, such as the input device 1600. In one or more embodiments, a user may employ an input device 1600 (which may be a separate computer or processor, a keyboard such as the keyboard 1210, a mouse such as the mouse 1211, a microphone, a screen or display 1209 (e.g., a touch screen or display), or any other input device known to those skilled in the art). In one or more system embodiments, an input device 1600 may not be used (e.g., where user interaction is eliminated by one or more artificial intelligence features discussed herein). In one or more system embodiments, the output device 1601 may receive one or more outputs discussed herein to perform coregistration, navigation planning, autonomous navigation, movement detection, control, and/or any other process discussed herein. In one or more system embodiments, the database 1603 and/or the memory 1602 may have outputted information (e.g., trained model(s), detected marker information, image data, test data, validation data, training data, coregistration result(s), segmentation model information, object detection/regression model information, combination model information, etc.) stored therein. That said, one or more embodiments may include several types of data stores, memory, storage media, etc. as discussed above, and such storage media, memory, data stores, etc. may be stored locally or remotely.

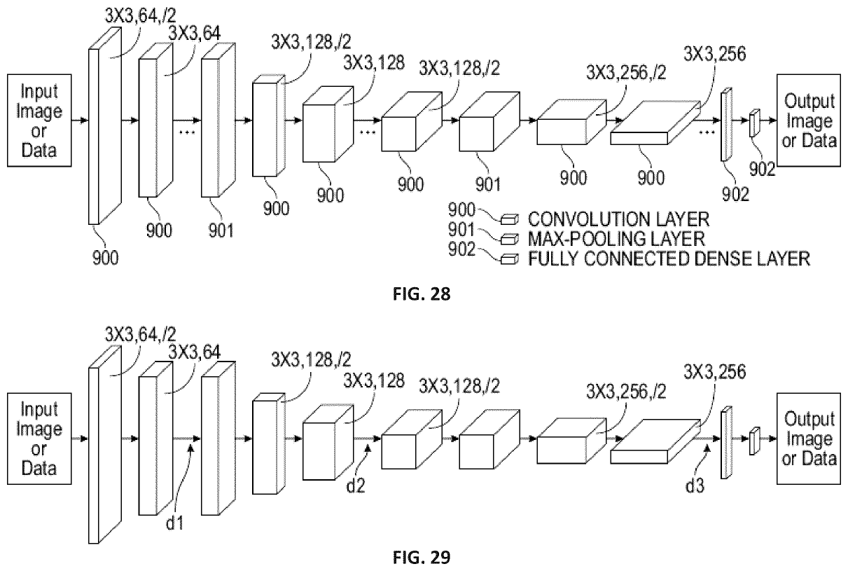

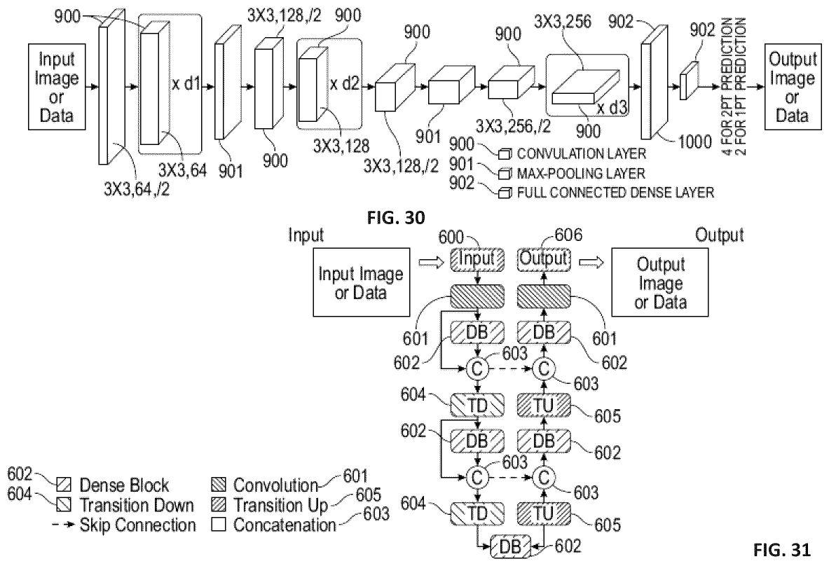

For regression model(s), the input may be the entire image frame(s), and the output may be the centroid coordinates of a target, an octagon, circle or other geometric shape used or discussed herein, one or more airways, and/or coordinates of a portion of a catheter or probe. As shown diagrammatically in FIGS. 28-30, an example of an input image on the left side of FIGS. 28-30 and a corresponding output image on the right side of FIGS. 28-30 are illustrated for regression model(s). At least one architecture of a regression model is shown in FIG. 28. In at least the embodiment of FIG. 28, the regression model may use a combination of one or more convolution layers 900, one or more max-pooling layers 901, and one or more fully connected dense layers 902. While not limited to the Kernel size, Width/Number of filters (output size), and Stride sizes shown for each layer (e.g., in the left convolution layer of FIG. 28, the Kernel size is “3×3”, the Width/# of filters (output size) is “64”, and the Stride size is “2”). Another hyper-parameter search with a fixed optimizer and with a different width may be performed, and at least one embodiment example of a model architecture for a convolutional neural network for this scenario is shown in FIG. 29. One or more embodiments may use one or more features for a regression model as discussed in “Deep Residual Learning for Image Recognition” to Kaiming He, et al., Microsoft Research, Dec. 10, 2015 (https://arxiv.org/pdf/1512.03385.pdf), which is incorporated by reference herein in its entirety. FIG. 30 shows at least a further embodiment example of a created architecture of/for a regression model(s).

Since the output from a segmentation model, in one or more embodiments, is a “probability” of each pixel that may be categorized as a target or as an estimate (incorrect) or actual (correct) match, post-processing after prediction via the trained segmentation model may be developed to better define, determine, or locate the final coordinate of catheter location and/or determine the navigation planning, autonomous navigation, movement detection, and/or control status of the catheter or continuum robot. One or more embodiments of a semantic segmentation model may be performed using the One-Hundred Layers Tiramisu method discussed in “The One Hundred Layers Tiramisu: Fully Convolutional DenseNets for Semantic Segmentation” to Simon Jégou, et al., Montreal Institute for Learning Algorithms, published Oct. 31, 2017 (https://arxiv.org/pdf/1611.09326.pdf), which is incorporated by reference herein in its entirety. A segmentation model may be used in one or more embodiment, for example, as shown in FIG. 31. At least one embodiment may utilize an input 600 as shown to obtain an output 606 of at least one embodiment of a segmentation model method. For example, by applying the One-Hundred Layers Tiramisu method(s), one or more features, such as, but not limited to, convolution 601, concatenation 603, transition up 605, transition down 604, dense block 602, etc., may be employed by slicing the training data set. While not limited to only or by only these embodiment examples, in one or more embodiments, a slicing size may be one or more of the following: 100×100, 224×224, 512×512. A batch size (of images in a batch) may be one or more of the following: 2, 4, 8, 16, and, from the one or more experiments performed, a bigger batch size typically performs better (e.g., with greater accuracy). In one or more embodiments, 16 images/batch may be used. The optimization of all of these hyper-parameters depends on the size of the available data set as well as the available computer/computing resources; thus, once more data is available, different hyper-parameter values may be chosen. Additionally, in one or more embodiments, steps/epoch may be 100, and the epochs may be greater than (>) 1000. In one or more embodiments, a convolutional autoencoder (CAE) may be used.

The present disclosure and/or one or more components of devices, systems, and storage mediums, and/or methods, thereof also may be used in conjunction with continuum robot devices, systems, methods, and/or storage mediums and/or with endoscope devices, systems, methods, and/or storage mediums. Such continuum robot devices, systems, methods, and/or storage mediums are disclosed in at least: U.S. Pat. No. 11,882,365, issued on Jan. 23, 2024, the disclosure of which is incorporated by reference herein in its entirety. Such endoscope devices, systems, methods, and/or storage mediums are disclosed in at least: U.S. Pat. Pub. No. 2022/0202502 A1, published on Jun. 30, 2022, the disclosure of which is incorporated by reference herein in its entirety; and U.S. Pat. Pub. No. 2022/0202274 A1, published on Jun. 30, 2022, the disclosure of which is incorporated by reference herein in its entirety. Any of the features of the present disclosure may be used in combination with any of the features as discussed in U.S. Pat. Pub. No. 2024/0112407 A1, published Apr. 4, 2024, the disclosure of which is incorporated by reference herein in its entirety. Any of the features of the present disclosure may be used in combination with any of the features as discussed in U.S. Pat. Pub. No. 2023/0131269, published on Apr. 26, 2023, the disclosure of which is incorporated by reference herein in its entirety.

An imaging apparatus or system, such as, but not limited to, a robotic bronchoscope and/or imaging devices or systems, discussed herein may have or include three bendable sections. The visualization technique(s) and methods discussed herein may be used with one or more imaging apparatuses, systems, methods, or storage mediums of U.S. Pat. Pub. No. 2024/0112407 A1, published Apr. 4, 2024, the disclosure of which is incorporated by reference herein in its entirety.

Further, the present disclosure and/or one or more components of devices, systems, and storage mediums, and/or methods, thereof also may be used in conjunction with continuum robotic systems and catheters, such as, but not limited to, those described in U.S. Patent Publication Nos. 2019/0105468; 2021/0369085; 2020/0375682; 2021/0121162; 2021/0121051; and 2022-0040450, each of which patents and/or patent publications are incorporated by reference herein in their entireties.

The present disclosure and/or one or more components of devices, systems, and storage mediums, and/or methods, thereof also may be used in conjunction with autonomous robot devices, systems, methods, and/or storage mediums and/or with endoscope devices, systems, methods, and/or storage mediums. Such continuum robot devices, systems, methods, and/or storage mediums are disclosed in at least: PCT/US2024/025546, filed on Apr. 19, 2024, which is incorporated by reference herein in its entirety.

The present disclosure and/or one or more parts of devices, systems, and storage mediums, and/or methods, thereof also may be used in conjunction with autonomous robot devices, systems, methods, and/or storage mediums and/or with endoscope devices, systems, methods, and/or storage mediums, and/or other features that may be used with same, such as, but not limited to, any of the features disclosed in at least: International Patent Application No. PCT/US2024/037935, filed Jul. 12, 2024, the disclosure of which is incorporated by reference herein in its entirety; International Patent Application No. PCT/US2024/037924, the disclosure of which is incorporated by reference herein in its entirety; and International Patent Application No. PCT/US2024/037930, the disclosure of which is incorporated by reference herein in its entirety.

Although the disclosure herein has been described with reference to particular features and/or embodiments, it is to be understood that these features and/or embodiments are merely illustrative of the principles and applications of the present disclosure (and are not limited thereto), and the scope of the present disclosure is not limited to the disclosed features and/or embodiments. It is therefore to be understood that numerous modifications may be made to the illustrative features and/or embodiments and that other arrangements may be devised without departing from the spirit and scope of the present disclosure. Indeed, the present disclosure encompasses and includes any combination of any of the feature(s) and/or embodiment(s) (or component(s) thereof) discussed herein. The scope of the following claims is to be accorded the broadest interpretation so as to encompass all such modifications, equivalent structures, and functions.

1. A continuum robot for performing driving/navigation and/or driving/navigation planning, the continuum robot comprising:
one or more processors that operate to:
detect one or more airways in an image or frame or in a view of a camera;
set an airway of the one or more airways to be aimed;
detect a target point in the set airway; and
determine whether a predetermined insertion depth or end point is reached and whether the detected target point is within a catheter bending threshold range for a catheter of or in communication with the continuum robot such that the driving/navigation and/or the driving/navigation planning is performed by simultaneously evaluating whether to: (i) bend one or more sections of the catheter; and (ii) move a translational stage of the continuum robot or in communication with the catheter of the continuum robot.

2. The continuum robot of claim 1, wherein the one or more processors further operate to one or more of the following:
(i) use direct communication to reduce a latency and increase a responsiveness for the direction/navigation and/or the direction/navigation planning;
(ii) operate the translational stage forwards or backwards and command bending of the one or more sections of the catheter simultaneously or separately;
(iii) provide more precision over when, where, and how motion commands are applied to the catheter and/or to the translational stage;
(iv) provide improved smoothness of motion and a reduction in navigation time by bending the one or more sections of the catheter and moving the translational stage simultaneously;
(v) address situations where the catheter may not be able to align with the target airway by employing retracting or backwards motion of the catheter;
(vi) reduce or minimize situations where control command(s) is/are sent based on an incorrect airway;
(vii) identify situations which affect autonomous control, the situations including breathing motion and entering a new generation for one or more airways; and/or
(viii) improve alignment timing and accuracy by sending a larger motion in a case where the target point is beyond a predetermined distance and by sending a more refined motion in a case where the target point is within or equal to a predetermined distance.

3. The continuum robot of claim 1, wherein the one or more processors further operate to one or more of the following:
(i) compute a direction vector from a center of a depth map to a center of the target detected airway;
(ii) compute a direction vector from a center of a depth map to a center of the target detected airway where the direction vector operates to inform a virtual gamepad controller or other controller and/or the one or more processors for bending a tip of the catheter, for moving the translational stage, and/or for moving the one or more sections of the catheter;
(iii) compute a direction vector from a center of a depth map to a center of the target detected airway, and advance the continuum robot in a straight line for engagement of the translational stage in a case where the direction vector has a magnitude that is less than 30% of a width of the image or frame or of the view of the camera;
(iv) repeat, for each image or frame of a plurality of images or frames or for each view of the camera for a plurality of views, the determination of whether the predetermined insertion depth or end point is reached and whether the detected target point is within the catheter bending threshold range for the catheter such that the driving/navigation and/or the driving/navigation planning is performed by simultaneously evaluating whether to: (a) bend one or more sections of the catheter; and (b) move the translational stage of the continuum robot or in communication with the catheter of the continuum robot; and/or
(v) maintain a set or predetermined/calculated linear speed and a set or predetermined/calculated bending speed.

4. The continuum robot of claim 1, wherein the one or more processors further operate to one or more of the following:
(i) in a case where the target point is within the catheter bending threshold range, set a bending speed and a magnitude for the one or more sections of the catheter and send the bend command to the one or more sections of the catheter and repeat the detection of the airways in the image or frame or in the view of the camera, the setting of the airway to be aimed, the detection of the target point in the airway, and the determinations of whether the predetermined insertion depth or end point has been reached and whether the target point is within the catheter bending threshold range;
(ii) in a case where the target point is not within the catheter bending threshold range and in a case where the predetermined insertion depth or end point is reached, then the driving/navigation and/or the planning is complete and/or a sample is taken using the catheter;
(iii) in a case where the predetermined insertion depth or end point has not been reached, then determine whether the target point is within a stage motion threshold range for the translational stage;
(iv) in a case where the predetermined insertion depth or end point has not been reached and in a case where the target point is within a stage motion threshold range for the translational stage, then send a stage motion command to the translational stage and set a stage motion direction, speed, and magnitude for the motion of the translational stage, and repeat the detection of the airways in the image or frame or in the view of the camera, the setting of the airway to be aimed, the detection of the target point in the airway, and the determinations of whether the predetermined insertion depth or end point has been reached and whether the target point is within the catheter bending threshold range; and/or
(v) in a case where the predetermined insertion depth or end point has not been reached and in a case where the target point is not within a stage motion threshold range for the translational stage, then the process either waits until the target point is within the stage motion threshold range or intervention is used to get the target point within the stage motion threshold range or to find a target point that is within the stage motion threshold range.

5. The continuum robot of claim 1, wherein the one or more processors further operate to one or more of the following:
(i) employ one or more command set and communication techniques using direct communication to reduce latency, increase responsiveness, and to move the translational stage and/or the one or more sections of the catheter;
(ii) use a location of the target point within the image or frame or within the view of the camera to determine one or more control commands to be sent for movement of the translational stage and/or the one or more sections of the catheter; and/or
(iii) use a location of the target point within the image or frame or within the view of the camera to determine one or more control commands to be sent for movement of the translational stage and/or the one or more sections of the catheter, wherein the one or more control commands includes one or more of: gamepad commands, serial commands, and/or any other form of communication/message in which the one or more processors operate to convert the communication/message to catheter movement(s) and/or translational stage movement(s), including translational stage insertion/retraction, catheter tip bending, catheter tip displacement, or other bending/displacement/state change(s) for the one or more sections of the catheter.

6. The continuum robot of claim 1, wherein the one or more processors further operate to one or more of the following:
(i) use command thresholding, or use command thresholding to introduce more precision over when/where/how certain motion commands are applied, improve smoothness of motion and reduce navigation time by bending the catheter and moving the translational stage simultaneously, and address situations where a catheter may not be able to align with an airway by using retraction;
(ii) compute a direction vector from a center of a depth map to a center of the target detected airway where the direction vector operates to inform a virtual gamepad controller or other controller and/or the one or more processors for bending a tip of the catheter, for moving the translational stage, and/or for moving the one or more sections of the catheter;
(iii) compare a magnitude to a predetermined threshold to determine a case where to command the translational stage to move where the magnitude is on a first side of the predetermined threshold and to determine another case where to command bending of the one or more sections of the catheter where the magnitude is on a second side of the predetermined threshold;
(iv) compare a magnitude to a predetermined threshold to determine a case where to command the translational stage to move where the magnitude is on a first side of the predetermined threshold and to determine another case where to command bending of the one or more sections of the catheter where the magnitude is on a second side of the predetermined threshold, wherein the predetermined threshold is 30% of a width of the image or frame or of the view of the camera;
(v) compare a magnitude to one or more additional predetermined thresholds upon which no commands are sent in a case where the magnitude falls on a set or predetermined side of the one or more additional predetermined thresholds;
(vi) compare a magnitude to a predetermined threshold range, and use a linear or translational stage movement command or retraction command in a case where the magnitude is within the predetermined threshold range to use a retraction to bring the target point closer towards a center of the image or frame or of the view of the camera;
(vii) compare a magnitude to a predetermined threshold range, and use a linear or translational stage movement command or retraction command in a case where the magnitude is within the predetermined threshold range to use a retraction to bring the target point closer towards a center of the image or frame or of the view of the camera, wherein the retraction uses a reverse Follow-The-Leader (rFTL) algorithm or process to re-orient the one or more sections of the catheter in a manner in which there is less impedance in the bending used to align the catheter with the target airway; and/or
(viii) use one or more overlapping thresholds or threshold ranges, wherein insertion uses a maximum threshold of 35% of a width of the image or frame or of the view of the camera while bending has a minimum threshold of 25% of the width of the image or frame or of the view of the camera such that both the bending and the insertion are commanded in a case where the magnitude falls within 25% and 35% of the width of the image or frame or of the view of the camera and/or such that the bending and the insertion are performed using a Follow-The-Leader algorithm or process.

7. The continuum robot of claim 1, wherein the one or more processors further operate to one or more of the following:
(i) use target history technique(s) to reduce situations where control commands are sent based on an incorrect airway and/or to identify situations which affect autonomous control, including a breathing motion or entering a new generation of the one or more airways;
(ii) repeat, for each image or frame of a plurality of images or frames or for each view of the camera for a plurality of views, the determination of whether the predetermined insertion depth or end point is reached and whether the detected target point is within the catheter bending threshold range for the catheter such that the driving/navigation and/or the driving/navigation planning is performed by simultaneously evaluating whether to: (a) bend one or more sections of the catheter; and (b) move the translational stage of the continuum robot or in communication with the catheter of the continuum robot, with or without influence from previous one or more images or frames or from previous views of the camera;
(iii) consider a difference in a location of the target airway to an unselected airway of a previous image or frame or of a previous view of the camera to determine whether a misdetection of an airway has occurred, and, in a case where the difference exceeds a predetermined amount such that a large displacement has occurred, no movement is commanded until a next image or frame or a next view of the camera;
(iv) detect breathing motion in a case where a magnitude of a direction of a target airway increases beyond a predetermined range, and mute or subdue one or more control outputs or commands until the magnitude and/or the breathing motion returns to the acceptable predetermined range;
(v) determine that a new branch of airway has been entered in a case where the target airway near a center of the image or frame or of the view of the camera is no longer found in a later image or frame or a later view of the camera; and/or
(vi) use one or more thresholds that are one or more of: set or calculated, predetermined or fixed, adjusted manually from an input, and/or adjusted automatically based on prior changes or other one or more conditions.

8. The continuum robot of claim 1, wherein the one or more processors further operate to one or more of the following:
(i) use command speed and/or magnitude features or techniques to improve alignment timing and/or accuracy by sending a larger motion in a case where a target is further away than a predetermined threshold and by sending a more refined motion in a case where the target is closer than or equal to the predetermined threshold;
(ii) use command speed and/or magnitude features or techniques, where the speed and/or the magnitude of one or more motion commands depend on a magnitude of a direction vector;
(iii) use command speed and/or magnitude features or techniques, where command levels are proportional to a position of the target airway within a corresponding threshold, the threshold being or representing a gradient of speed and/or of magnitude of motion commands;
(iv) use command speed and/or magnitude features or techniques, where command levels are proportional to a position of the target airway within a corresponding threshold, the threshold being or representing a gradient of speed and/or of magnitude of motion commands, and the gradient is discrete or continuous while a slope or range size follows a mathematical formula or a parameterized function and/or the slope or range is defined manually or automatically;
(v) use command speed and/or magnitude features or techniques, where command levels are proportional to a position of the target airway within a corresponding threshold, the threshold being or representing a gradient of speed and/or of magnitude of motion commands, where the gradient has ranges within where a slope and/or size calculation different from other ranges;
(vi) use any or all possible commands that controllers accept and/or use any or all possible commands that control when, how, or where each individual command is activated or deactivates;
(vii) use previous state(s) for error detection and control determinations; and/or
(viii) react to the target point within one or more images or frames or within one or more views of a camera by sending or commanding a predetermined or set amount of motion.

9. The continuum robot of claim 1, wherein the one or more processors further operate to determine one or more geometry metrics, wherein the one or more geometry metrics is a depth map or maps obtained or generated by processing one or more images or frames or one or more view of the camera, and wherein the processors further operate to one or more of the following:
advance the continuum robot or catheter to the target point;
display one or more target points on a display;
detect one or more objects, and fit one or more set or predetermined geometric shapes or one or more circles, rectangles, squares, ovals, octagons, and/or triangles in or on one or more detected objects;
define the one or more target points based on the one or more set or predetermined geometric shapes or based on the one or more circles, rectangles, squares, ovals, octagons, and/or triangles of the one or more detected objects;
set a center or portion of the circle or circles as the one or more target points for a next movement of the continuum robot;
in a case where the one or more target points are not detected, then apply peak detection to the depth map or maps and use one or more detected peaks as the one or more targets; and/or
in a case where one or more peaks are not detected, then use a deepest point of the depth map or maps as the one or more targets.

10. The continuum robot of claim 9, wherein the one or more processors further operate to one or more of the following:
(i) estimate or determine the depth map or maps using artificial intelligence (AI) architecture, where the artificial intelligence architecture includes one or more of the following: a neural network, a convolutional neural network, a generative adversarial network (GAN), a consistent generative adversarial network (cGAN), a three cycle-consistent generative adversarial network (3cGAN), recurrent neural networks, and/or any other AI architecture discussed herein; and/or
(ii) use a neural network, a convolutional neural network, a generative adversarial network (GAN), a consistent generative adversarial network (cGAN), a three cycle-consistent generative adversarial network (3cGAN), recurrent neural networks, and/or any other AI architecture discussed herein to one or more of: select a target detection method or mode; use or evaluate a depth map or depth maps; perform the selected target detection method or mode; identify one or more target points; evaluate the accuracy of the identified one or more target points; and/or plan the navigation of the continuum robot, autonomously move the continuum robot to the one or more target points, or display the one or more target points on one of the one or more images, frames, or views on a display or indicate on the display or via an indicator on the display or via a light or indicator on the continuum robot that the navigation plan has been planned or determined.

11. The continuum robot of claim 1, wherein the target point is located in a lung or in an airway, and the continuum robot is a bronchoscope.

12. The continuum robot of claim 1, wherein the one or more processors further operate to perform registration or co-registration for changes due to movement, breathing, or any other change that may occur during imaging or a procedure with the continuum robot.

13. The continuum robot of claim 1, wherein the continuum robot is a steerable catheter with a camera at a distal end of the steerable catheter.

14. The continuum robot of claim 1, wherein the continuum robot includes one or more of the following:
(i) a distal bending section or portion, wherein the distal bending section or portion is commanded or instructed automatically or based on an input of a user of the continuum robot;
(ii) a plurality of bending sections or portions including a distal or most distal bending portion or section and the rest of the plurality of the bending sections or portions; and/or
(iii) the one or more processors further operate to instruct or command the forward motion, or the motion in the set or predetermined direction, of the translational stage and/or of the continuum robot automatically or autonomously and/or based on an input of a user of the continuum robot.

15. The continuum robot of claim 14, further comprising:
a base and an actuator that operates to bend the plurality of the bending sections or portions independently; and
the translational stage being a motorized linear stage and/or a sensor or camera that operates to move the continuum robot forward and backward, and/or in the predetermined or set direction or directions,
wherein the one or more processors operate to control the actuator and the motorized linear stage and/or the sensor or camera.

16. The continuum robot of claim 1, further comprising a user interface of or disposed on a base, or disposed remotely from a base, the user interface operating to receive an input from a user of the continuum robot to move one or more of the plurality of bending sections or portions and/or a motorized linear stage as the translational stage and/or a sensor or camera, wherein the one or more processors further operate to receive the input from the user interface, and the one or more processors and/or the user interface operate to use a base coordinate system.

17. The continuum robot of claim 1, wherein the plurality of bending sections or portions each include driving wires that operate to bend a respective section or portion of the plurality of sections or portions, wherein the driving wires are connected to an actuator so that the actuator operates to bend one or more of the plurality of bending sections or portions using the driving wires.

18. The continuum robot of claim 1, wherein one or more of the following:
(i) the continuum robot further comprises an operational controller or joystick that operates to issue or input one or more commands or instructions as an input to the one or more processors, the input including an instruction or command to move one or more of a plurality of bending sections or portions and/or a motorized linear stage and/or a sensor or camera;
(ii) the continuum robot further includes a display to display one or more images taken by the continuum robot and/or to display a navigation plan and/or an autonomous navigation path of the continuum robot; and/or
(iii) the continuum robot further comprises an operational controller or joystick that operates to issue or input one or more commands or instructions to one or more processors, the input including an instruction or command to move one or more of a plurality of bending sections or portions and/or a motorized linear stage and/or a sensor or camera, and the operational controller or joystick operates to be controlled by a user of the continuum robot.

19. A method for performing navigation and/or navigation planning for a continuum robot, the method comprising:
detecting one or more airways in an image or frame or in a view of a camera;
setting an airway of the one or more airways to be aimed;
detecting a target point in the set airway; and
determining whether a predetermined insertion depth or end point is reached and whether the detected target point is within a catheter bending threshold range for a catheter of or in communication with the continuum robot such that the driving/navigation and/or the driving/navigation planning is performed by simultaneously evaluating whether to: (i) bend one or more sections of the catheter; and (ii) move a translational stage of the continuum robot or in communication with the catheter of the continuum robot.

20. The method of claim 19, further comprising one or more of the following:
(i) using direct communication to reduce a latency and increase a responsiveness for the direction/navigation and/or the direction/navigation planning;
(ii) operating the translational stage forwards or backwards and command bending of the one or more sections of the catheter simultaneously or separately;
(iii) providing more precision over when, where, and how motion commands are applied to the catheter and/or to the translational stage;
(iv) providing improved smoothness of motion and a reduction in navigation time by bending the one or more sections of the catheter and moving the translational stage simultaneously;
(v) addressing situations where the catheter may not be able to align with the target airway by employing retracting or backwards motion of the catheter;
(vi) reducing or minimizing situations where control command(s) is/are sent based on an incorrect airway;
(vii) identifying situations which affect autonomous control, the situations including breathing motion and entering a new generation for one or more airways; and/or
(viii) improving alignment timing and accuracy by sending a larger motion in a case where the target point is beyond a predetermined distance and by sending a more refined motion in a case where the target point is within or equal to a predetermined distance.

21. The method of claim 19, further comprising one or more of the following:
(i) computing a direction vector from a center of a depth map to a center of the target detected airway;
(ii) computing a direction vector from a center of a depth map to a center of the target detected airway where the direction vector operates to inform a virtual gamepad controller or other controller and/or the one or more processors for bending a tip of the catheter, for moving the translational stage, and/or for moving the one or more sections of the catheter;
(iii) computing a direction vector from a center of a depth map to a center of the target detected airway, and advance the continuum robot in a straight line for engagement of the translational stage in a case where the direction vector has a magnitude that is less than 30% of a width of the image or frame or of the view of the camera;
(iv) repeating, for each image or frame of a plurality of images or frames or for each view of the camera for a plurality of views, the determination of whether the predetermined insertion depth or end point is reached and whether the detected target point is within the catheter bending threshold range for the catheter such that the driving/navigation and/or the driving/navigation planning is performed by simultaneously evaluating whether to: (a) bend one or more sections of the catheter; and (b) move the translational stage of the continuum robot or in communication with the catheter of the continuum robot; and/or
(v) maintaining a set or predetermined/calculated linear speed and a set or predetermined/calculated bending speed.

22. The method of claim 19, further comprising one or more of the following:
(i) in a case where the target point is within the catheter bending threshold range, setting a bending speed and a magnitude for the one or more sections of the catheter and sending the bend command to the one or more sections of the catheter and repeating the detection of the airways in the image or frame or in the view of the camera, the setting of the airway to be aimed, the detection of the target point in the airway, and the determinations of whether the predetermined insertion depth or end point has been reached and whether the target point is within the catheter bending threshold range;
(ii) in a case where the target point is not within the catheter bending threshold range and in a case where the predetermined insertion depth or end point is reached, then determining that the target point has been reached or retract the catheter until the target point is within the catheter bending threshold range;
(iii) in a case where the predetermined insertion depth or end point has not been reached, then determining whether the target point is within a stage motion threshold range for the translational stage;
(iv) in a case where the predetermined insertion depth or end point has not been reached and in a case where the target point is within a stage motion threshold range for the translational stage, then sending a stage motion command to the translational stage and setting a stage motion direction, speed, and magnitude for the motion of the translational stage, and repeating the detection of the airways in the image or frame or in the view of the camera, the setting of the airway to be aimed, the detection of the target point in the airway, and the determinations of whether the predetermined insertion depth or end point has been reached and whether the target point is within the catheter bending threshold range; and/or
(v) in a case where the predetermined insertion depth or end point has not been reached and in a case where the target point is not within a stage motion threshold range for the translational stage, then waiting until the target point is within the stage motion threshold range, using an intervention to get the target point within the stage motion threshold range, or finding a target point that is within the stage motion threshold range; and/or
(vi) in a case where the predetermined insertion depth or end point has been reached and in a case where the target point is not within the catheter bending threshold range, then completing the driving/navigation and/or the planning and/or taking a sample using the catheter.

23. The method of claim 19, further comprising one or more of the following:
(i) employing one or more command set and communication techniques using direct communication to reduce latency, increase responsiveness, and to move the translational stage and/or the one or more sections of the catheter;
(ii) using a location of the target point within the image or frame or within the view of the camera to determine one or more control commands to be sent for movement of the translational stage and/or the one or more sections of the catheter; and/or
(iii) using a location of the target point within the image or frame or within the view of the camera to determine one or more control commands to be sent for movement of the translational stage and/or the one or more sections of the catheter, wherein the one or more control commands includes one or more of: gamepad commands, serial commands, and/or any other form of communication/message in which the one or more processors operate to convert the communication/message to catheter movement(s) and/or translational stage movement(s), including translational stage insertion/retraction, catheter tip bending, catheter tip displacement, or other bending/displacement/state change(s) for the one or more sections of the catheter.

24. The method of claim 19, further comprising one or more of the following:
(i) using command thresholding, or using command thresholding to introduce more precision over when/where/how certain motion commands are applied, improve smoothness of motion and reduce navigation time by bending and moving a stage simultaneously, and address situations where a catheter may not be able to align with an airway by using retraction;
(ii) computing a direction vector from a center of a depth map to a center of the target detected airway where the direction vector operates to inform a virtual gamepad controller or other controller and/or the one or more processors for bending a tip of the catheter, for moving the translational stage, and/or for moving the one or more sections of the catheter;
(iii) comparing a magnitude to a predetermined threshold to determine a case where to command the translational stage to move where the magnitude is on a first side of the predetermined threshold and to determine another case where to command bending of the one or more sections of the catheter where the magnitude is on a second side of the predetermined threshold;
(iv) comparing a magnitude to a predetermined threshold to determine a case where to command the translational stage to move where the magnitude is on a first side of the predetermined threshold and to determine another case where to command bending of the one or more sections of the catheter where the magnitude is on a second side of the predetermined threshold, wherein the predetermined threshold is 30% of a width of the image or frame or of the view of the camera;
(v) comparing a magnitude to one or more additional predetermined thresholds upon which no commands are sent in a case where the magnitude falls on a set or predetermined side of the one or more additional predetermined thresholds;
(vi) comparing a magnitude to a predetermined threshold range, and using a linear or translational stage movement command or retraction command in a case where the magnitude is within the predetermined threshold range to use a retraction to bring the target point closer towards a center of the image or frame or of the view of the camera;
(vii) comparing a magnitude to a predetermined threshold range, and using a linear or translational stage movement command or retraction command in a case where the magnitude is within the predetermined threshold range to use a retraction to bring the target point closer towards a center of the image or frame or of the view of the camera, wherein the retraction uses a reverse Follow-The-Leader (rFTL) algorithm or process to re-orient the one or more sections of the catheter in a manner in which there is less impedance in the bending used to align the catheter with the target airway; and/or
(viii) using one or more overlapping thresholds or threshold ranges, wherein insertion uses a maximum threshold of 35% of a width of the image or frame or of the view of the camera while bending has a minimum threshold of 25% of the width of the image or frame or of the view of the camera such that both the bending and the insertion are commanded in a case where the magnitude falls within 25% and 35% of the width of the image or frame or of the view of the camera and/or such that the bending and the insertion are performed using a Follow-The-Leader algorithm or process.

25. The method of claim 19, further comprising one or more of the following:
(i) using target history technique(s) to reduce situations where control commands are sent based on an incorrect airway and/or to identify situations which affect autonomous control, including a breathing motion or entering a new generation of the one or more airways;
(ii) repeating, for each image or frame of a plurality of images or frames or for each view of the camera for a plurality of views, the determination of whether the predetermined insertion depth or end point is reached and whether the detected target point is within the catheter bending threshold range for the catheter such that the driving/navigation and/or the driving/navigation planning is performed by simultaneously evaluating whether to: (a) bend one or more sections of the catheter; and (b) move the translational stage of the continuum robot or in communication with the catheter of the continuum robot, with or without influence from previous one or more images or frames or from previous views of the camera;
(iii) considering a difference in a location of the target airway to an unselected airway of a previous image or frame or of a previous view of the camera to determine whether a misdetection of an airway has occurred, and, in a case where the difference exceeds a predetermined amount such that a large displacement has occurred, no movement is commanded until a next image or frame or a next view of the camera;
(iv) detecting breathing motion in a case where a magnitude of a direction of a target airway increases beyond a predetermined range, and muting or subduing one or more control outputs or commands until the magnitude and/or the breathing motion returns to the acceptable predetermined range;
(v) determining that a new branch of airway has been entered in a case where the target airway near a center of the image or frame or of the view of the camera is no longer found in a later image or frame or a later view of the camera; and/or
(vi) using one or more thresholds that are one or more of: set or calculated, predetermined or fixed, adjusted manually from an input, and/or adjusted automatically based on prior changes or other one or more conditions.

26. The method of claim 19, further comprising one or more of the following:
(i) using command speed and/or magnitude features or techniques to improve alignment timing and/or accuracy by sending a larger motion in a case where a target is further away than a predetermined threshold and by sending a more refined motion in a case where the target is closer than or equal to the predetermined threshold;
(ii) using command speed and/or magnitude features or techniques, where the speed and/or the magnitude of one or more motion commands depend on a magnitude of a direction vector;
(iii) using command speed and/or magnitude features or techniques, where command levels are proportional to a position of the target airway within a corresponding threshold, the threshold being or representing a gradient of speed and/or of magnitude of motion commands;
(iv) using command speed and/or magnitude features or techniques, where command levels are proportional to a position of the target airway within a corresponding threshold, the threshold being or representing a gradient of speed and/or of magnitude of motion commands, and the gradient is discrete or continuous while a slope or range size follows a mathematical formula or a parameterized function and/or the slope or range is defined manually or automatically;
(v) using command speed and/or magnitude features or techniques, where command levels are proportional to a position of the target airway within a corresponding threshold, the threshold being or representing a gradient of speed and/or of magnitude of motion commands, where the gradient has ranges within where a slope and/or size calculation different from other ranges;
(vi) using any or all possible commands that controllers accept and/or use any or all possible commands that control when, how, or where each individual command is activated or deactivates;
(vii) using previous state(s) for error detection and control determinations; and/or
(viii) reacting to the target point within one or more images or frames or within one or more views of a camera by sending or commanding a predetermined or set amount of motion.

27. The method of claim 19, further comprising determining one or more geometry metrics, wherein the one or more geometry metrics is a depth map or maps obtained or generated by processing one or more images or frames or one or more view of the camera, and wherein the method further comprises one or more of the following:
advancing the continuum robot or catheter to the target point;
displaying one or more target points on a display;
detecting one or more objects, and fitting one or more set or predetermined geometric shapes or one or more circles, rectangles, squares, ovals, octagons, and/or triangles in or on one or more detected objects;
defining the one or more target points based on the one or more set or predetermined geometric shapes or based on the one or more circles, rectangles, squares, ovals, octagons, and/or triangles of the one or more detected objects;
setting a center or portion of the circle or circles as the one or more target points for a next movement of the continuum robot;
in a case where the one or more target points are not detected, then applying peak detection to the depth map or maps and use one or more detected peaks as the one or more targets; and/or
in a case where one or more peaks are not detected, then using a deepest point of the depth map or maps as the one or more targets.

28. The method of claim 27, further comprising one or more of the following:
(i) estimating or determining the depth map or maps using artificial intelligence (AI) architecture, where the artificial intelligence architecture includes one or more of the following: a neural network, a convolutional neural network, a generative adversarial network (GAN), a consistent generative adversarial network (cGAN), a three cycle-consistent generative adversarial network (3cGAN), recurrent neural networks, and/or any other AI architecture discussed herein; and/or
(ii) using a neural network, a convolutional neural network, a generative adversarial network (GAN), a consistent generative adversarial network (cGAN), a three cycle-consistent generative adversarial network (3cGAN), recurrent neural networks, and/or any other AI architecture discussed herein to one or more of: select a target detection method or mode; use or evaluate a depth map or depth maps; perform the selected target detection method or mode; identify one or more target points; evaluate the accuracy of the identified one or more target points; and/or plan the navigation of the continuum robot, autonomously move the continuum robot to the one or more target points, or display the one or more target points on one of the one or more images, frames, or views on a display or indicate on the display or via an indicator on the display or via a light or indicator on the continuum robot that the navigation plan has been planned or determined.

29. A non-transitory computer-readable storage medium storing at least one program for causing a computer to execute a method for performing navigation and/or navigation planning for a continuum robot, the method comprising:
detecting one or more airways in an image or frame or in a view of a camera;
setting an airway of the one or more airways to be aimed;
detecting a target point in the set airway; and
determining whether a predetermined insertion depth or end point is reached and whether the detected target point is within a catheter bending threshold range for a catheter of or in communication with the continuum robot such that the driving/navigation and/or the driving/navigation planning is performed by simultaneously evaluating whether to: (i) bend one or more sections of the catheter; and (ii) move a translational stage of the continuum robot or in communication with the catheter of the continuum robot.
